SEQUENCE LISTING (154 sequences)

SEQ ID NO: 1 - DNA, 69 bp, synthetic construct
  misc_feature 1..69: note = Synthetic DNA
  source 1..69: mol_type = other DNA
gctgaacttgtaggcctgataagcgcagcgtatcaggcaatttttataatcttcatttaaatggcgcgc

SEQ ID NO: 2 - DNA, 69 bp, synthetic construct
  misc_feature 1..69: note = Synthetic DNA
  source 1..69: mol_type = other DNA
gcgcaacgcaattaatgtgagttagctcactcattaggcaccccaggcttcgcctacctgtgacggaag

SEQ ID NO: 3 - DNA, 75 bp, synthetic construct
  misc_feature 1..75: note = Synthetic DNA
  source 1..75: mol_type = other DNA
cgcttaaagatgcctaatccgccaacggcttacattttacttattgaggtgaatagtgtaggctggagctgcttc

SEQ ID NO: 4 - DNA, 66 bp, synthetic construct
  misc_feature 1..66: note = synthetic DNA
  source 1..66: mol_type = other DNA
ggcgtttgtcatcagagccaaccacgtccgcagacgtggttgctatcatatgaatatcctccttag

SEQ ID NO: 5 - DNA, 62 bp, synthetic construct
  misc_feature 1..62: note = synthetic DNA
  source 1..62: mol_type = other DNA
taatgcgccgccagtaaatcaacatgaaatgccgctggctccgtgtaggctggagctgcttc

SEQ ID NO: 6 - DNA, 70 bp, synthetic construct
  misc_feature 1..70: note = synthetic DNA
  source 1..70: mol_type = other DNA
ccaacaacaagcactggataaagcgagtctgcgtcgcctggttcagttcacatatgaatatcctccttag

SEQ ID NO: 7 - DNA, 70 bp, synthetic construct
  misc_feature 1..70: note = synthetic DNA
  source 1..70: mol_type = other DNA
aaaatacatctggcacgttgaggtgttaacgataataaaggaggtagcaagtgtaggctggagctgcttc

SEQ ID NO: 8 - DNA, 70 bp, synthetic construct
  misc_feature 1..70: note = synthetic DNA
  source 1..70: mol_type = other DNA
cctccagataaaaaaacggggccaaaaggccccggtagtgtacaacagtccatatgaatatcctccttag

SEQ ID NO: 9 - DNA, 70 bp, synthetic construct
  misc_feature 1..70: note = synthetic DNA
  source 1..70: mol_type = other DNA
gtttggcgtttgtcatcagagccaaccacgtccgcagacgtggttgctatgtgtaggctggagctgcttc

SEQ ID NO: 10 - DNA, 70 bp, synthetic construct
  misc_feature 1..70: note = Synthetic DNA
  source 1..70: mol_type = other DNA
ttgtcattgttggatgcgacgctcaagcgtcgcatcaggcataaagcagacttaagcgacttcattcacc

SEQ ID NO: 11 - DNA, 70 bp, synthetic construct
  misc_feature 1..70: note = Synthetic DNA
  source 1..70: mol_type = other DNA
catggcggtaatgcgccgccagtaaatcaacatgaaatgccgctggctccgtgtaggctggagctgcttc

SEQ ID NO: 12 - DNA, 70 bp, synthetic construct
  misc_feature 1..70: note = synthetic DNA
  source 1..70: mol_type = other DNA
ccaacaacaagcactggataaagcgagtctgcgtcgcctggttcagttcacttaagcgacttcattcacc

SEQ ID NO: 13 - DNA, 70 bp, synthetic construct; identical to SEQ ID NO: 7
  misc_feature 1..70: note = synthetic DNA
  source 1..70: mol_type = other DNA

SEQ ID NO: 14 - DNA, 70 bp, synthetic construct
  misc_feature 1..70: note = synthetic DNA
  source 1..70: mol_type = other DNA
cctccagataaaaaaacggggccaaaaggccccggtagtgtacaacagtccttaagcgacttcattcacc

SEQ ID NO: 15 - DNA, 70 bp, synthetic construct
  misc_feature 1..70: note = synthetic DNA
  source 1..70: mol_type = other DNA
gcgcaacgcaattaatgtgagttagctcactcattaggcaccccaggcttgtgtaggctggagctgcttc

SEQ ID NO: 16 - DNA, 70 bp, synthetic construct
  misc_feature 1..70: note = synthetic DNA
  source 1..70: mol_type = other DNA
gctgaacttgtaggcctgataagcgcagcgtatcaggcaatttttataatcttaagcgacttcattcacc

SEQ ID NO: 17 - DNA, 70 bp, synthetic construct
  misc_feature 1..70: note = synthetic DNA
  source 1..70: mol_type = other DNA
caaaaagcggtcaaattatacggtgcgcccccgtgatttcaaacaataaggtgtaggctggagctgcttc

SEQ ID NO: 18 - DNA, 70 bp, synthetic construct
  misc_feature 1..70: note = synthetic DNA
  source 1..70: mol_type = other DNA
ataacgtggctttttttggtaagcagaaaataagtcattagtgaaaatatcttaagcgacttcattcacc

SEQ ID NO: 19 - DNA, 20 bp, synthetic construct
  misc_feature 1..20: note = synthetic DNA
  source 1..20: mol_type = other DNA
caggtttcccgactggaaag

SEQ ID NO: 20 - DNA, 20 bp, synthetic construct
  misc_feature 1..20: note = synthetic DNA
  source 1..20: mol_type = other DNA
tgtgcgtcgttgggctgatg

SEQ ID NO: 21 - DNA, 20 bp, synthetic construct
  misc_feature 1..20: note = synthetic DNA
  source 1..20: mol_type = other DNA
cgcttgtcattgttggatgc

SEQ ID NO: 22 - DNA, 22 bp, synthetic construct
  misc_feature 1..22: note = synthetic DNA
  source 1..22: mol_type = other DNA
gctgacaaagtgcgatttgttc

SEQ ID NO: 23 - DNA, 20 bp, synthetic construct
  misc_feature 1..20: note = synthetic DNA
  source 1..20: mol_type = other DNA
gtcgccctgtaattcgtaac

SEQ ID NO: 24 - DNA, 20 bp, synthetic construct
  misc_feature 1..20: note = synthetic DNA
  source 1..20: mol_type = other DNA
ctttcggtcagaccaccaac

SEQ ID NO: 25 - DNA, 20 bp, synthetic construct
  misc_feature 1..20: note = synthetic DNA
  source 1..20: mol_type = other DNA
acgcctctgatttggcaaag

SEQ ID NO: 26 - DNA, 20 bp, synthetic construct
  misc_feature 1..20: note = synthetic DNA
  source 1..20: mol_type = other DNA
agccagtgcgcttaataacc

SEQ ID NO: 27 - DNA, 20 bp, synthetic construct
  misc_feature 1..20: note = synthetic DNA
  source 1..20: mol_type = other DNA
gctgaacagcaatccacttg

SEQ ID NO: 28 - DNA, 20 bp, synthetic construct
  misc_feature 1..20: note = synthetic DNA
  source 1..20: mol_type = other DNA
tgaacgatatggtgagctgg

SEQ ID NO: 29 - DNA, 500 bp, synthetic construct
  misc_feature 1..500: note = synthetic DNA
  source 1..500: mol_type = other DNA
agatggaatggcgattttcactctaaattttaaaaattgcctctttacaatagcgaatttcctaacccttttttttttttgttgattgcctattgctcgttcacttcccatttattttctctcgaatttcaccaaaagttgatgtggataatcaatcatcgggcctattcctgcgggtaaaacgcagggcccaactcaggatagggtttaatattattttagaggacttacaagaaggaagttatatggtttaaaaattgtaacaaagttagaacacatttatttagcaggtctaatttagggctgcaactatctttttggttattcatataaaatataattttttatttatatagagaatacaagtggaatcatctttaacgccagcttgtagtgcgcattgcagaataatggaagttcaaaaattaaaagcgaaggagaagtgatagtagaaagacggatgggaggctgggggacgaagagaaagtaaaagggttaat

SEQ ID NO: 30 - DNA, 500 bp, synthetic construct
  misc_feature 1..500: note = synthetic DNA
  source 1..500: mol_type = other DNA
ttttttataaaagttgctgttgatttgctcgagaacttattgcttatttggccctgataactatataagaaaagaaatacagttattccttgtttatgctggctttttgtccactttttctcaactatataactatgatgttggaaaggacaccggttctgtaactttgcagtgaaaataagtgtgatggatgactgagaatgctttcttgtaagcgaaaagaagtacgtgttccaaaaataaagcagaaaggcgaaaagggtcgaatgtaagacactaaataaatattttaagaagaggaaaagtcgcctcagaaacgctaaaatgcatccgatttcccaaagaggaagtctaatgttttcgatttgtgaaaaaaagataaaaatcgaagaaaatgtagggagccgcgcgttacccggattgatatttgagtgatcgacggcgtcacaaaagaaagaatgcttggctaatcaagaaaagtatgtggtttgtttcatcta

SEQ ID NO: 31 - DNA, 20 bp, synthetic construct
  misc_feature 1..20: note = synthetic DNA
  source 1..20: mol_type = other DNA
gcaggtctaatttagggctg

SEQ ID NO: 32 - DNA, 20 bp, synthetic construct
  misc_feature 1..20: note = synthetic DNA
  source 1..20: mol_type = other DNA
agaaagcattctcagtcatc

SEQ ID NO: 33 - DNA, 1000 bp, synthetic construct
  misc_feature 1..1000: note = synthetic DNA
  source 1..1000: mol_type = other DNA
gactgcaagatttcggcctgggcggacgggaatcgtcagttttgtaatttctgtatcaatgattttcatggtctcttcctcaagtccgagccggtcgtattgcttgccctgctcccagagttcaagattcatgacaatcgtgattcgtttattgcttctgaccgcgccagcgccaaatagcgtcatcacattgataatgccaaggcccctgatctcaagaaggtgctcaattaattccggagcgtttcccacaagagtatcctgatcctcctgccgtatttcaacgcaatcatcggcaacaaggcgatgccctcttttcacaagctctagcgctgtttcgctttttccgacgccgctttttcctgtgatcagcacgccgacaccatatatatcgacaagaacgccatgaattgctgtggtaggcgccagcctgctctcaaggaagttggttaaacggcttgacagtcttgtcgttttcagcggcgatctgaggacaggcaccccatttttctcggaggcgtcaatcagctcctgcgggatgggcatatctctagaaagaataatagctggtgttacatcagtgcacagagaatccattcgctgctttttctcctcttcaggaagctgttcaaagaaagaaagctctgtttttccgagaagctgcacgcgctccctcgggtaatatgtaaaatatccggcaatttcaatacctggtcttgataggtcactcattgtaatcgggcggttaattccttcttctccgctgattaattccaaattgaactgttccattacgtcttttgtgcgaacctttgccacgatatgttcctcctgttccgggctgccccgagcttgctcacaatactttcattttatcactttcgggcttgaacctaaaacagattttataaaaggggggaaaacacctcagctggtctagatcactagtctgaaaaagagtaaaataaaggtattcaaattccagaaaggcggatcatct

SEQ ID NO: 34 - DNA, 1000 bp, synthetic construct
  misc_feature 1..1000: note = synthetic DNA
  source 1..1000: mol_type = other DNA
aaggaacatgctgacttatgaatatcaataaacaatcgcctattccgatttactatcagattatggagcaattaaaaacccaaattaagaacggagagctgcagccggatatgcctcttccttctgagcgcgaatatgccgaacaattcgggatcagccggatgacagttcgccaggcgctttctaatttagttaatgaaggcttgctctatcgcctgaaagggcggggcacctttgtcagcaagccaaaaatggaacaagcacttcaagggctgacaagctttaccgaggatatgaaaagccgcgggatgacaccgggcagcaggctcattgattatcagcttattgattcaactgaggagctcgcggctatattaggctgcgggcacccctcctctatccataaaatcactcgggtgcggctggcaaatgatattccgatggcgattgagtcctcacatattccgtttgagcttgcgggtgaattgaacgaatcgcattttcagtcgtcgatctatgatcatattgaaaggtacaacagcataccgatttcccgtgcaaaacaggagcttgagccaagcgctgccaccacggaagaagcgaatattcttggtattcaaaagggagcgcctgtcctattaattaaacgaacaacatatctgcagaacggaactgcttttgagcatgcaaaatccgtatacagaggcgaccgttatacatttgtccactatatggatcgtctttcataaaaaaagcctccaaccctttttaaggattggagacatggcgaaaatcaaactggtctggtgccggacgatatgtttcttttttcgtcttgaacttccagatcggtgatttcgttttgccgttaaaactgtcttccactataatgtaccaataataaacagactgcggttcaagatgatcccagcggaattcagctgtgtccccgctcttcacttgctcccgttttccgagctcttcattggtatatacgtta

SEQ ID NO: 35 - DNA, 1000 bp, synthetic construct
  misc_feature 1..1000: note = synthetic DNA
  source 1..1000: mol_type = other DNA
tggcggacatggaataaatcacaaacgacaaagatgacgccggcaagaatagagttaatcaaatagagcacgggcgcaacgaacaagaaagaaaactcaaccggttctgtaattccggtcagcatagatgtgagcgccgcagaaatcatcacgccggagatcatttttttcttttccggacgcgcggtatggataatggcaagagcaacggccggcagacagaaaatcatgtaagggaaatcccccatcataaagcgcccggctgtcgggtctcccgcgaaaaaccttgtcaggtcgccggttacggtgttgcctgttgatgggtctgtgtattctcccatcataaaatagaaaggcgtataaaaaatatgatgcaggccaaaaggaatcagcaaacgatagatcgttgcataaaagaacaggccgactgttgaatcggcaattaaactgctggctgcgttaattccgttttggatcagcggccaaacgaatgagaaaatgacgccgatcaccaatgaactgacggaagtaatgatcgggacaaagcgttttccagagaaaaatccaaggaccggatgcagctcgattgatgaaaatcgcttatataaataggcggcgagaagcccgataatgattcctccgaaaacccccatatcaatcaggtgctcggctccttcatacggaggctgaaggccgagtaattttcccatattgtcgagggtgacggttaaaattaagtatccgatgacagcggcaagtccggctacaccttctccgccggctaatccgatcgcgacccccacggcgaaaatcagcggaaggttatcgaatacaacgccgcccgcatcctttataatagggatgttcagtaaatccttgtctccgaaacggagcaaaagacctgctgccggcaggacggcaaccggagtcatcaacgcgcggccaagctgctgcagaatttgaaatgcctttttaaacatgacagtctccttttattgtg

SEQ ID NO: 36 - DNA, 1000 bp, synthetic construct
  misc_feature 1..1000: note = synthetic DNA
  source 1..1000: mol_type = other DNA
gtgacaccccctcaaagagatagacaagcaccatatttgttatgaccaatttatgatacttgtcattacgaatttagcaccgcccttatcaaactgtcaatattaatttctgaaaatttgttataaaagaaggatacaaatctttcatattgggagggcaaatggtattatggtctcaatgaaaaagaacggattgcatacagaatggggagaatgaaatgacagctttatattctgttatcaagtttaaaatcattgagttaattaaatcgggcaaatatcaggcgaatgatcagctgccgacggagagtgagttttgcgaacaatatgatgtcagcagaacaactgtgagactggctctgcagcagctagagcttgagggatatattaaaagaattcaaggaaaagggacatttgtatcggcggccaaaatacaaacgccgattccgcataagattacgagctttgcagaacaaatgagaggacttcgttctgaatcaaaagtgcttgagcttgtggtgattcctgccgatcattccatcgccgagcttttgaaaatgaaagagaatgaacctgtcaacaagcttgtcagagtcagatacgccgagggggaacctttgcagtatcatacctcatatattccctggaaggcggcaccggggctggcgcaggaggaatgcaccggctcgctgtttgaattgttaaggacaaaatacaatattgaaatcagcaggggcacggaatcgatcgaaccgattttaacggatgaaacgatcagcggacacttattaaccaatgtcggagcgcctgcgtttttatcagaatcccttacctatgataaaaatgaagaagtggtggaatatgcgcaaattattacacggggagaccgaacgaaattcaccgtagaacagtcatatcattcataaagcaatgtgttttaagaagggaatggtggttctatgtttttatttacgaatggaaaagtgctgtggggagcagt

SEQ ID NO: 37 - DNA, 20 bp, synthetic construct
  misc_feature 1..20: note = synthetic DNA
  source 1..20: mol_type = other DNA
tgtaatcgggcggttaattc

SEQ ID NO: 38 - DNA, 19 bp, synthetic construct
  misc_feature 1..19: note = synthetic DNA
  source 1..19: mol_type = other DNA
gccctttcaggcgatagag

SEQ ID NO: 39 - DNA, 20 bp, synthetic construct
  misc_feature 1..20: note = synthetic DNA
  source 1..20: mol_type = other DNA
acggcgaaaatcagcggaag

SEQ ID NO: 40 - DNA, 20 bp, synthetic construct
  misc_feature 1..20: note = synthetic DNA
  source 1..20: mol_type = other DNA
tcactctccgtcggcagctg

SEQ ID NO: 41 - DNA, 72 bp, synthetic construct
  misc_feature 1..72: note = UTR1
  source 1..72: mol_type = other DNA
aattcgccggagggatattaaaatgaatggaaaattgaaacatcttaatcatgctaaggaggttttctaatg

SEQ ID NO: 42 - protein, 237 aa, Escherichia coli
MRKITQAISAVCLLFALNSSAVALASSPSPLNPGTNVARLAEQAPIHWVSVAQIENSLAGRPPMAVGFDIDDTVLFSSPGFWRGKKTFSPESEDYLKNPVFWEKMNNGWDEFSIPKEVARQLIDMHVRRGDAIFFVTGRSPTKTETVSKTLADNFHIPATNMNPVIFAGDKPGQNTKSQWLQDKNIRIFYGDSDNDITAARDVGARGIRILRASNSTYKPLPQAGAFGEEVIVNSEY

SEQ ID NO: 43 - protein, 272 aa, Escherichia coli
MARLAAFDMDGTLLMPDHHLGEKTLSTLARLRERDITLTFATGRHALEMQHILGALSLDAYLITGNGTRVHSLEGELLHRDDLPADVAELVLYQQWDTRASMHIFNDDGWFTGKEIPALLQAFVYSGFRYQIIDVKKMPLGSVTKICFCGDHDDLTRLQIQLYEALGERAHLCFSATDCLEVLPVGCNKGAALTVLTQHLGLSLRDCMAFGDAMNDREMLVSVGSGFIMGNAMPQLRAELPHLPVIGHCRNQAVSHYLTHWLDYPHLPYSPE

SEQ ID NO: 44 - protein, 355 aa, Escherichia coli
MSQKYLFIDRDGTLISEPPSDFQVDRFDKLAFEPGVIPELLKLQKAGYKLVMITNQDGLGTQSFPQADFDGPHNLMMQIFTSQGVQFDEVLICPHLPADECDCRKPKVKLVERYLAEQAMDRANSYVIGDRATDIQLAENMGITGLRYDRETLNWPMIGEQLTRRDRYAHVVRNTKETQIDVQVWLDREGGSKINTGVGFFDHMLDQIATHGGFRMEINVKGDLYIDDHHTVEDTGLALGEALKIALGDKRGICRFGFVLPMDECLARCALDISGRPHLEYKAEFTYQRVGDLSTEMIEHFFRSLSYTMGVTLHLKTKGKNDHHRVESLFKAFGRTLRQAIRVEGDTLPSSKGVL

SEQ ID NO: 45 - protein, 266 aa, Escherichia coli
MTEPLTETPELSAKYAWFFDLDGTLAEIKPHPDQVVVPDNILQGLQLLATASDGALALISGRSMVELDALAKPYRFPLAGVHGAERRDINGKTHIVHLPDAIARDISVQLHTVIAQYPGAELEAKGMAFALHYRQAPQHEDALMTLAQRITQIWPQMALQQGKCVVEIKPRGTSKGEAIAAFMQEAPFIGRTPVFLGDDLTDESGFAVVNRLGGMSVKIGTGATQASWRLAGVPDVWSWLEMITTALQQKRENNRSDDYESFSRSI

SEQ ID NO: 46 - protein, 253 aa, Escherichia coli
MRILLSNDDGVHAPGIQTLAKALREFADVQVVAPDRNRSGASNSLTLESSLRTFTFENGDIAVQMGTPTDCVYLGVNALMRPRPDIVVSGINAGPNLGDDVIYSGTVAAAMEGRHLGFPALAVSLDGHKHYDTAAAVTCSILRALCKEPLRTGRILNINVPDLPLDQIKGIRVTRCGTRHPADQVIPQQDPRGNTLYWIGPPGGKCDAGPGTDFAAVDEGYVSITPLHVDLTAHSAQDVVSDWLNSVGVGTQW

SEQ ID NO: 47 - protein, 191 aa, Escherichia coli
MAKSVPAIFLDRDGTINVDHGYVHEIDNFEFIDGVIDAMRELKKMGFALVVVTNQSGIARGKFTEAQFETLTEWMDWSLADRDVDLDGIYYCPHHPQGSVEEFRQVCDCRKPHPGMLLSARDYLHIDMAASYMVGDKLEDMQAAVAANVGTKVLVRTGKPITPEAENAADWVLNSLADLPQAIKKQQKPAQ

SEQ ID NO: 48 - protein, 219 aa, Escherichia coli [sequence omitted]

SEQ ID NO: 49 - protein, 271 aa, Escherichia coli
MFSIQQPLLVFSDLDGTLLDSHSYDWQPAAPWLTRLREANVPVILCSSKTSAEMLYLQKTLGLQGLPLIAENGAVIQLAEQWQEIDGFPRIISGISHGEISLVLNTLREKEHFKFTTFDDVDDATIAEWTGLSRSQAALTQLHEASVTLIWRDSDERMAQFTARLNELGLQFMQGARFWHVLDASAGKDQAANWIIATYQQLSGKRPTTLGLGDGPNDAPLLEVMDYAVIVKGLNREGVHLHDEDPARVWRTQREGPEGWREGLDHFFSAR

SEQ ID NO: 50 - protein, 216 aa, Escherichia coli
MRCKGFLFDLDGTLVDSLPAVERAWSNWARRHGLAPEEVLAFIHGKQAITSLRHFMAGKSEADIAAEFTRLEHIEATETEGITALPGAIALLSHLNKAGIPWAIVTSGSMPVARARHKIAGLPAPEVFVTAERVKRGKPEPDAYLLGAQLLGLAPQECVVVEDAPAGVLSGLAAGCHVIAVNAPADTPRLNEVDLVLHSLEQITVTKQPNGDVIIQ

SEQ ID NO: 51 - protein, 270 aa, Escherichia coli
MAIKLIAIDMDGTLLLPDHTISPAVKNAIAAARARGVNVVLTTGRPYAGVHNYLKELHMEQPGDYCITYNGALVQKAADGSTVAQTALSYDDYRFLEKLSREVGSHFHALDRTTLYTANRDISYYTVHESFVATIPLVFCEAEKMDPNTQFLKVMMIDEPAILDQAIARIPQEVKEKYTVLKSAPYFLEILDKRVNKGTGVKSLADVLGIKPEEIMAIGDQENDIAMIEYAGVGVAMDNAIPSVKEVANFVTKSNLEDGVAFAIEKYVLN

SEQ ID NO: 52 - protein, 238 aa, Escherichia coli
MRFYRPLGRISALTFDLDDTLYDNRPVILRTEREALTFVQNYHPALRSFQNEDLQRLRQAVREAEPEIYHDVTRWRFRSIEQAMLDAGLSAEEASAGAHAAMINFAKWRSRIDVPQQTHDTLKQLAKKWPLVAITNGNAQPELFGLGDYFEFVLRAGPHGRSKPFSDMYFLAAEKLNVPIGEILHVGDDLTTDVGGAIRSGMQACWIRPENGDLMQTWDSRLLPHLEISRLASLTSLI

SEQ ID NO: 53 - protein, 199 aa, Escherichia coli
MLYIFDLGNVIVDIDFNRVLGAWSDLTRIPLASLKKSFHMGEAFHQHERGEISDEAFAEALCHEMALPLSYEQFSHGWQAVFVALRPEVIAIMHKLREQGHRVVVLSNTNRLHTTFWPEEYPEIRDAADHIYLSQDLGMRKPEARIYQHVLQAEGFSPSDTVFFDDNADNIEGANQLGITSILVKDKTTIPDYFAKVLC

SEQ ID NO: 54 - protein, 222 aa, Escherichia coli
MSTPRQILAAIFDMDGLLIDSEPLWDRAELDVMASLGVDISRRNELPDTLGLRIDMVVDLWYARQPWNGPSRQEVVERVIARAISLVEETRPLLPGVREAVALCKEQGLLVGLASASPLHMLEKVLTMFDLRDSFDALASAEKLPYSKPHPQVYLDCAAKLGVDPLTCVALEDSVNGMIASKAARMRSIVVPAPEAQNDPRFVLADVKLSSLTELTAKDLLG

SEQ ID NO: 55 - protein, 188 aa, Escherichia coli
MYERYAGLIFDMDGTILDTEPTHRKAWREVLGHYGLQYDIQAMIALNGSPTWRIAQAIIELNQADLDPHALAREKTEAVRSMLLDSVEPLPLVDVVKSWHGRRPMAVGTGSESAIAEALLAHLGLRHYFDAVVAADHVKHHKPAPDTFLLCAQRMGVQPTQCVVFEDADFGIQAARAAGMDAVDVRLL

SEQ ID NO: 56 - protein, 188 aa, Escherichia coli
MSKAGASLATCYGPVSADVIAKAENIRLLILDVDGVLSDGLIYMGNNGEELKAFNVRDGYGIRCALTSDIEVAIITGRKAKLVEDRCATLGITHLYQGQSNKLIAFSDLLEKLAIAPENVAYVGDDLIDWPVMEKVGLSVAVADAHPLLIPRADYVTRIAGGRGAVREVCDLLLLAQGKLDEAKGQSI

SEQ ID NO: 57 - protein, 223 aa, Pseudomonas putida
MRLRAVLFDMDGTLLDTAPDFIAICQAMLAERGLPAVDDNLIRGVISGGARAMVATAFAMDPEADGFEALRLEFLERYQRDCAVHSKLFEGMAELLADIEKGNLLWGVVTNKPVRFAEPIMQQLGLAERSALLICPDHVKNSKPDPEPLILACKTLNLDPASVLFVGDDLRDIESGRDAGTRTAAVRYGYIHPEDNPNNWGADVVVDHPLELRKVIDSALCGC

SEQ ID NO: 58 - protein, 432 aa, Escherichia coli
MKAILIPFLSLLIPLTPQSAFAQSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAILSRAGGSIADFTGHRQTAFRELERVLNFPQSNLCLKREKQDESCSLTQALPSELKVSADNVSLTGAVSLASMLTEIFLLQQAQGMPEPGWGRITDSHQWNTLLSLHNAQFYLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 59 - protein, 252 aa, Escherichia coli
MNKFEDIRGVAFDLDGTLVDSAPGLAAAVDMALYALELPVAGEERVITWIGNGADVLMERALTWARQERATQRKTMGKPPVDDDIPAEEQVRILRKLFDRYYGEVAEEGTFLFPHVADTLGALQAKGLPLGLVTNKPTPFVAPLLEALDIAKYFSVVIGGDDVQNKKPHPDPLLLVAERMGIAPQQMLFVGDSRNDIQAAKAAGCPSVGLTYGYNYGEAIDLSQPDVIYQSINDLLPALGLPHSENQESKND

SEQ ID NO: 60 - protein, 322 aa, Escherichia coli [sequence omitted]

SEQ ID NO: 61 - protein, 250 aa, Escherichia coli
MTIKNVICDIDGVLMHDNVAVPGAAEFLHGIMDKGLPLVLLTNYPSQTGQDLANRFATAGVDVPDSVFYTSAMATADFLRRQEGKKAYVVGEGALIHELYKAGFTITDVNPDFVIVGETRSYNWDMMHKAAYFVANGARFIATNPDTHGRGFYPACGALCAGIEKISGRKPFYVGKPSPWIIRAALNKMQAHSEETVIVGDNLRTDILAGFQAGLETILVLSGVSSLDDIDSMPFRPSWIYPSVAEIDVI

SEQ ID NO: 62 - protein, 272 aa, Escherichia coli
MTTRVIALDLDGTLLTPKKTLLPSSIEALARAREAGYQLIIVTGRHHVAIHPFYQALALDTPAICCNGTYLYDYHAKTVLEADPMPVIKALQLIEMLNEHHIHGLMYVDDAMVYEHPTGHVIRTSNWAQTLPPEQRPTFTQVASLAETAQQVNAVWKFALTHDDLPQLQHFGKHVEHELGLECEWSWHDQVDIARGGNSKGKRLTKWVEAQGWSMENVVAFGDNFNDISMLEAAGTGVAMGNADDAVKARANIVIGDNTTDSIAQFIYSHLI

SEQ ID NO: 63 - protein, 271 aa, Escherichia coli
MSVKVIVTDMDGTFLNDAKTYNQPRFMAQYQELKKRGIKFVVASGNQYYQLISFFPELKDEISFVAENGALVYEHGKQLFHGELTRHESRIVIGELLKDKQLNFVACGLQSAYVSENAPEAFVALMAKHYHRLKPVKDYQEIDDVLFKFSLNLPDEQIPLVIDKLHVALDGIMKPVTSGFGFIDLIIPGLHKANGISRLLKRWDLSPQNVVAIGDSGNDAEMLKMARYSFAMGNAAENIKQIARYATDDNNHEGALNVIQAVLDNTSPFNS

SEQ ID NO: 64 - protein, 561 aa, Escherichia coli
MNINVAELLNGNYILLLFVVLALGLCLGKLRLGSIQLGNSIGVLVVSLLLGQQHFSINTDALNLGFMLFIFCVGVEAGPNFFSIFFRDGKNYLMLALVMVGSALVIALGLGKLFGWDIGLTAGMLAGSMTSTPVLVGAGDTLRHSGMESRQLSLALDNLSLGYALTYLIGLVSLIVGARYLPKLQHQDLQTSAQQIARERGLDTDANRKVYLPVIRAYRVGPELVAWTDGKNLRELGIYRQTGCYIERIRRNGILANPDGDAVLQMGDEIALVGYPDAHARLDPSFRNGKEVFDRDLLDMRIVTEEVVVKNHNAVGKRLAQLKLTDHGCFLNRVIRSQIEMPIDDNVVLNKGDVLQVSGDARRVKTIADRIGFISIHSQVTDLLAFCAFFVIGLMIGMITFQFSTFSFGMGNAAGLLFAGIMLGFMRANHPTFGYIPQGALSMVKEFGLMVFMAGVGLSAGSGINNGLGAIGGQMLIAGLIVSLVPVVICFLFGAYVLRMNRALLFGAMMGARTCAPAMEIISDTARSNIPALGYAGTYAIANVLLTLAGTIIVMVWPGLG

SEQ ID NO: 65 - protein, 199 aa, Escherichia coli
MKQSHFFAHLSRLKLINRWPLMRNVRTENVSEHSLQVAMVAHALAAIKNRKFGGNVNAERIALLAMYHDASEVLTGDLPTPVKYFNSQIAQEYKAIEKIAQQKLVDMVPEELRDIFAPLIDEHAYSDEEKSLVKQADALCAYLKCLEELAAGNNEFLLAKTRLEATLEARRSQEMDYFMEIFVPSFHLSLDEISQDSPL

SEQ ID NO: 66 - protein, 221 aa, Escherichia coli
MSRIEAVFFDCDGTLVDSEVICSRAYVTMFQEFGITLDPEEVFKRFKGVKLYEIIDIVSLEHGVTLAKTEAEHVYRAEVARLFDSELEAIEGAGALLSAITAPMCVVSNGPNNKMQHSMGKLNMLHYFPDKLFSGYDIQRWKPDPALMFHAAKAMNVNVENCILVDDSVAGAQSGIDAGMEVFYFCADPHNKPIVHPKVTTFTHLSQLPELWKARGWDITA

SEQ ID NO: 67 - protein, 604 aa, Escherichia coli
MSKISDLNYSQHITLADNFKQKSEVLNTWRVGMNDFARIAGGQDNRRNILSPGAFLEFLAKIFTLGYVDFSKRSNEAGRNMMAHIKSSSYSKDTNGNEKMKFYMNNPVGERADSPKVIIEISLSTITTMGTRQGHTAIIFPQPDGSTNRYEGKSFERKDESSLHLITNKVLACYQSEANKKIARLLNNNQELNNLQKLNNLQKLNNLLKLNNIQGLNNPQELNNPQNLNDSQELNNSQELNSPQELNDPQELNNSQDLNNSKVSCTVSVDSTITGLLKEPLNNALLAIRNEHLLLMPHVCDESISYLLGEKGILEEIDKLYALNDHGIDNDKVGNNEINDIKVNLSHILIDSLDDAKVNLTPVIDSILETFSKSPYINDVRILDWCFNKSMQYFDDTKKIKHACSVINHINLRSDQSKIAETLFFNLDKEPYKNSPELQGLIWNKLVVYVNEFNLSNREKTNLIQRLFDNVESIFNEVPVSILVNDIFMNDFFMKNPEMINWYFPQLLKSYEGEKIYFDNLKYDLNDNDKESNKEILKNQPDNVIKEKLNNEYKLRFRMMQTILQSRVNVLPYINEQRLNKLNPPENLRIAIEHFGWKNRPITA

SEQ ID NO: 68 - protein, 225 aa, Escherichia coli
MKWDWIFFDADETLFTFDSFTGLQRMFLDYSVTFTAEDFQDYQAVNKPLWVDYQNGAITSLQLQHGRFESWAERLNVEPGKLNEAFINAMAEICTPLPGAVSLLNAIRGNAKIGIITNGFSALQQVRLERTGLRDYFDLLVISEEVGVAKPNKKIFDYALEQAGNPDRSRVLMVGDTAESDILGGINAGLATCWLNAHHREQPEGIAPTWTVSSLHELEQLLCKH

SEQ ID NO: 69 - protein, 222 aa, Escherichia coli
MHINIAWQDVDTVLLDMDGTLLDLAFDNYFWQKLVPETWGAKNGVTPQEAMEYMRQQYHDVQHTLNWYCLDYWSEQLGLDICAMTTEMGPRAVLREDTIPFLEALKASGKQRILLTNAHPHNLAVKLEHTGLDAHLDLLLSTHTFGYPKEDQRLWHAVAEATGLKAERTLFIDDSEAILDAAAQFGIRYCLGVTNPDSGIAEKQYQRHPSLNDYRRLIPSLM

SEQ ID NO: 70 - protein, 421 aa, Escherichia coli
MASTFTSDTLPADHKAAIRQMKHALRAQLGDVQQIFNQLSDDIATRVAEINALKAQGDAVWPVLSYADIKAGHVTAEQREQIKRRGCAVIKGHFPREQALGWDQSMLDYLDRNRFDEVYKGPGDNFFGTLSASRPEIYPIYWSQAQMQARQSEEMANAQSFLNRLWTFESDGKQWFNPDVSVIYPDRIRRRPPGTTSKGLGAHTDSGALERWLLPAYQRVFANVFNGNLAQYDPWHAAHRTEVEEYTVDNTTKCSVFRTFQGWTALSDMLPGQGLLHVVPIPEAMAYVLLRPLLDDVPEDELCGVAPGRVLPVSEQWHPLLIEALTSIPKLEAGDSVWWHCDVIHSVAPVENQQGWGNVMYIPAAPMCEKNLAYAHKVKAALEKGASPGDFPREDYETNWEGRFTLADLNIHGKRALGMDV

SEQ ID NO: 71 - protein, 246 aa, Saccharomyces cerevisiae
MAEFSADLCLFDLDGTIVSTTVAAEKAWTKLCYEYGVDPSELFKHSHGARTQEVLRRFFPKLDDTDNKGVLALEKDIAHSYLDTVSLIPGAENLLLSLDVDTETQKKLPERKWAIVTSGSPYLAFSWFETILKNVGKPKVFITGFDVKNGKPDPEGYSRARDLLRQDLQLTGKQDLKYVVFEDAPVGIKAGKAMGAITVGITSSYDKSVLFDAGADYVVCDLTQVSVVKNNENGIVIQVNNPLTRA

SEQ ID NO: 72 - protein, 272 aa, Bacillus subtilis
MRIMASHDTPVSPAGILIDLDGTVFRGNELIEGAREAIKTLRRMGKKIVFLSNRGNISRAMCRKKLLGAGIETDVNDIVLSSSVTAAFLKKHYRFSKVWVLGEQGLVDELRLAGVQNASEPKEADWLVISLHETLTYDDLNQAFQAAAGGARIIATNKDRSFPNEDGNAIDVAGMIGAIETSAQAKTELVVGKPSWLMAEAACTAMGLSAHECMIIGDSIESDIAMGKLYGMKSALVLTGSAKQGEQRLYTPDYVLDSIKDVTKLAEEGILI

SEQ ID NO: 73-77 - skipped (5 entries)

SEQ ID NO: 78 - protein, 272 aa, Shigella flexneri
MARLAAFDMDGTLLMPDHHLGEKTLSTLARLRERDITLTFATGRHALEMQHILGALSLDAYLITGNGTRVHSLEGELLHRDDLPADVAELVLYQQWDTRASMHIFNDDGWFTGKEIPALLQAFVYSGFRYQIIDVKKMPLGSVTKICFCGDHDDLTRLQIQLYEALGERAHLCFSATDCLEVLPVGCNKGAALTVLTQHLDLSLRDCMAFGDAMNDREMLVSVGSGFIMGNAMPQLRAELPHLPVIGHCRNQAVSHYLTHWLDYPHLPYSPE

SEQ ID NO: 79 - protein, 272 aa, Shigella boydii
MARLAAFDMDGTLLMPDHHLGEKTLSTLARLRERDITLTFATGRHALEMQHILGALSLDAYLITGNGTRVHSLEGELLHRDDLPADVAELVLYQQWDTRASMHIFNDDGWFTGKEIPALLQAFVYNGFRYQIIDVKKMPLGSVTKICFCGDHDDLTRLQIQLYEALGERAHLCFSATDCLEVLPVGCNKGAALTVLTQHLGLSLRDCMAFGDAMNDREMLGSVGSGFIMGNAMPQLRAELPHLPVIGHCRNQAVSHYLTHWLDYPHLPYSPE

SEQ ID NO: 80 - protein, 272 aa, Escherichia fergusonii
MARLAAFDMDGTLLMPDHHLGEKTLSTLARLRERDITLTFATGRHALEMQHILGALSLDAYLITGNGTRVHSLEGELLHRDDLPADVAELVLYQQWDTRASMHIFNDDGWFTGKEIPALLQAFVYSGFRYQIIDVKKMPLDRVTKICFCGDHDDLTRLQIQLHEALGERAHLCFSATDCLEVLPVGCNKGAALTVLTQHLGLSLRECMAFGDAMNDREMLGSVGSGFIMGNAMPQLRAELPHLPVIGHCRNQAVSHYLTHWLDYPHLPYSPE

SEQ ID NO: 81 - protein, 245 aa, Staphylococcus aureus
MARLAAFDMDGTLLVPDHHLGEKTLSTLARLRERDITLTFATGRHALEMQHILGALSLDAYLITGNGTRVHSLEGELLHRDDLPADVAELVLYQQWDTRASMHIFNDDGWFTGKEIPALLQAFVYSGFRYQIIDVKKMPLGSVTKICFCGDHDDLTRLQIQLYEALGERAHLCFSATDCLEVLPVGCNKGAALTVLTQHLGLSLRDCTAFGDAMNDREMLVSVGSGFIMGNAMPQLRAELPHLPV

SEQ ID NO: 82 - protein, 272 aa, unidentified
  REGION 1..272: note = Escherichia albertii
MARLAAFDMDGTLLMPDHHLGEKTLSTLARLRDRDITLTFATGRHALEMRHILGTFALDAYLITGNGTRVHSQEGDLLYRDDLPADVAERVLHQKWDTQASMHIFNDDGWFTGQEMPSLLQAFVYSGFRYQIIDVKKMPLDRVTKICFCGDHDDLTRLQIQLNEALGDRAHLCFSAINCLEVLPVGCNKGAALAVLADYLGFSLRDCMAFGDAMNDREMLGSVGNGFIMGNAMPQLLAELPHLPVIGHCRNQAVSHYLTHWLDNPHLPYSPE

SEQ ID NO: 83 - protein, 272 aa, Citrobacter amalonaticus
MARLAAFDMDGTLLMPNHLLGDETLSTLARLRERDITLTFATGRHVLEMRHILGTFSLDAFLITGNGTRIHSLEGEVLHRQDLEPAVAEIVLHQRWDTQASMHIFNDNGWFTGQEIPEMLHAHVYSGFRYQIVDVARIPADRVTKVCFCGDHDDLTRLKIQLEEVLGARAHLCFSAVDCLEVLPVGCNKGSALEVLSGHLGLSLAECMAFGDAMNDREMLGSVGRGLIMGNAMPQLIAELPHLPVIGHCRNQAVSHFLTHWLDYPNLPYSPE

SEQ ID NO: 84 - protein, 272 aa, Salmonella enterica
MARLAAFDMDGTLLMPDHHLGKETLATLARLRERDITLTFATGRHVLEMRHILGTLSLDAYLITGNGTRIHSLEGDVLHRQDLDPQVADTVMHHAWDTRASMHVFNDNGWFTGQEIPALLQAHVYSGFRYQVIDIKSIPAHQVTKICFCGDHDDLIRLRIQLNEALEERAHLCFSAVDCLEVLPLGCNKGSALAVLSNHLGLSLADCMAFGDAMNDREMLGSVGRGLIMGNAMPQLIAALPHLSVIGHCGNQAVSHFLTHWLDNPHLPYSPE

SEQ ID NO: 85 - protein, 220 aa, Escherichia coli
LGALSLDAYLITGNGTRVHSLEGELLHRDDLPADVAELVLYQQWDTRASMHIFNDDGWFTGKEIPALLQAFVYSGFRYQIIDVKKMPLGSVTKICFCGDHDDLTRLQIQLYEALGERAHLCFSATDCLEVLPVGCNKGAALTVLTQHLGLSLRDCMAFGDAMNDREMLGSVGSGFIMGNAMPQLRAELPHLPVIGHCRNQAVSHYLTHWLDYPHLPYSPE

SEQ ID NO: 86 - protein, 355 aa, Shigella flexneri
MSQKYLFIDRDGTLISEPPSDFQVDRFDKLAFEPGVIPELLKLQKAGYKLVMITNQDGLGTQSFPQADFDGPHNLMMQIFTSQGVQFDEVLICPHLPADECDCRKPKVKLVERYLAEQAMDRANSYVIGDRATDIQLAENMGITGLRYDRETLNWPMIGEQLTKRDRYAHVVRNTKETQIDVQVWLDREGGSKINTGVGFFDHMLDQIATHGGFRMEINVKGDLYIDDHHTVEDTGLALGEALKIALGDKRGICRFGFVLPMDECLARCALDISGRPHLEYKAEFTYQRVGDLSTEMIEHFFRSLSYTMGVTLHLKTKGKNDHHRVESLFKAFGRTLRQAMRVEGDTLPSSKGVL

SEQ ID NO: 87 - protein, 355 aa, Escherichia albertii
MSQKYLFIDRDGTLISEPPSDFQVDRFDKLAFEPGVIPELLKLQKAGYKLVMITNQDGLGTQSFPQADFDGPHNLMMQIFTSQGVQFDEVLICPHLPADECDCRKPKVKLVERYLAEQAMDRANSYVIGDRATDIQLAENMGITGLRYDRETLNWPMIGEQLTKRDRYAHVVRNTKETQIDVQVWLDREGDSKINTGVGFFDHMLDQIATHGGFRMEINVKGDLYIDDHHTVEDTGLALGEALKIALGDKRGICRFGFVLPMDECLARCAMDISGRPHLEYKAEFTYQRVGDLSTEMIEHFFRSLSYTMGVTLHLKTKGKNDHHRVESLFKAFGRTLRQAIRVEGDTLPSSKGVL

SEQ ID NO: 88 - protein, 355 aa, Shigella flexneri
MSQKYLFIDRDGTLISEPPSDFQVDRFDKLAFEPGVIPELLKLQKAGYKLVMITNQDGLGTQSFPQADFDGPHNLMMQIFTSQGVQFDEVLICPHLPADECDCRKPKVKLVEGYLAEQAMDRANSYVIGDRATDIQLAENMGINGLRYDRETLNWPMIGEQLTKRDRYAHVVRNTKETQIDVQVWLDREGGSKINTGVGFFDHMLDQIATHGGFRMEINVKGDLYIDDHHTVEDTGLALGEALKIALGDKRGICRFGFVLPMDECLARCALDISGRPHLEYKAEFTYQRVGDLSTEMIEHFFRSLSYTMGVTLHLKTKGKNDHHRVESLFKAFGRTLRQALRVEGDTLPSSKGVL

SEQ ID NO: 89 - protein, 355 aa, Shigella sonnei
MSQKYLFIDRDGTLISEPPSDFQVDRFDKLAFEPGVIPELLKLQKAGYKLVMITNQDGLGTQSFPQADFDGPHNLMMQIFTSQGVQFDEVLICPPLPADECDCRKPKVKLVERYLAEQAMDRANSYVIGDRATDIQLAENMGINGLRYDREILNWPMIGEQLTKRDRYAHVVRNTKETQIDVQVWLDREGGSKINTGVGFFDHMLDQIATHGGFRMEINVKGDLYIDDHHTVEDTGLALGEALKMALGDKRGICRFGFVLPMDECLARCALDISGRPHLEYKAEFTYQRVGDLSTEMIEHFFRSLSYTMGVTLHLKTKGKNDHHRVESLFKAFGRTLRQAIRVEGDTLPSSKGVL

SEQ ID NO: 90 - protein, 355 aa, Escherichia coli
MSQKYLFIDRDGTLISEPPSDFQVDRFDKLAFEPGVIPQLLKLQKAGYKLVMITNQDGLGTQSFPQADFDGPHNLMMQIFTSQGVQFDEVLICPHLPSDECDCRKPKVKLVERYLAEQAMDRANSYVIGDRATDIQLAENMGINGLRYDRETLNWPMIGEQLIKRDRYAHVVRNTKETQIDVQVWLDREGGSKINTGVGFFDHMLDQIATHGGFRMEINVKGDLYIDDHHTVEDTGLALGEALKIALGDKRGICRFGFVLPMDECLARCALDISGRPHLEYKAEFTYQRVGDLSTEMIEHFFRSLSYTMGVTLHLKTKGKNDHHRVESLFKAFGRTLRQAIRVEGDTLPSSKGVL

SEQ ID NO: 91 - protein, 355 aa, Shigella dysenteriae
MSQKYLFIDRDGTLISEPPSDFQVDRFDKLAFEPGVIPELLKLQKAGYKLVMITNQDGLGTQSFPQADFDGPHNLMMQIFTSQGVQFDEVLICPHLPADECDCRKPKVKLVERYLAEQAMDRANSYVIGDRATDIQLAENMGINGLRYDCEILSWPMIGEQLTKRDRYAHVVRNTKETQIDVRVWLDREGGSKINTGVGFFDHMLDQIATHGGFRMEINVKGDLYIDDHHTVEDTGLALGEALKMALGDKRGICRFGFVLPMDECLARCALDISGRPHLEYKAEFTYQRVGDLSTEMIEHFFRSLSYTMGVTLHLKTKGKNDHHRVESLFKAFGRTLRQAIRVEGDTLPSSKGVL

SEQ ID NO: 92 - protein, 355 aa, Escherichia marmotae
MSQKYLFIDRDGTLISEPPSDFQVDRFDKLAFEPGVIPELLKLQKAGYKLVMITNQDGLGTQRFPQADFDGPHNLMMQIFTSQGVQFDEVLICPHLPDDECDCRKPKVKLVERYLAEQAMDRASSYVIGDRATDIQLAENMGINGLRYNRETLNWPMIGEQLTKRNRYAHVVRNTKETQIDVQVWLDREGGSKINTGVGFFDHMLDQIATHGGFRMEINVKGDLYIDDHHTVEDTGLALGEALKIALGDKRGICRFGFVLPMDECLARCALDISGRPHLEYKAEFTYQRVGDLSTEMIEHFFRSLSYTMGVTLHLKTKGKNDHHRVESLFKAFGRTLRQAIRVEGDTLPSSKGVL

SEQ ID NO: 93 - protein, 355 aa, Salmonella bongori
MSQKYLFIDRDGTLISEPPSDFQVDRFDKLAFEPEVIPVLLKLQKAGFKLVMITNQDGLGTQSFPQADFDGPHNLMMQVFTSQGVHFDEVLICPHLPADECDCRKPKIKLVERYLAEQAMDSANSYVIGDRATDVQLADNMGITGLRYHRETLNWSMIGEQLTKRDRYAHVIRNTKETQIDVRVWLDREGNSKINTGVGFFDHMLDQIATHGGFRMDVTVKGDLYIDDHHTVEDTGLALGEALKLALGDKRGICRFGFVLPMDECLARCALDISGRPHLEYKAEFTYQRVGDLSTEMIEHFFRSLSYTMGVTLHLKTKGKNDHHRVESLFKAFGRTLRQAIRVEGDTLPSSKGVL

SEQ ID NO: 94 - protein, 266 aa, Shigella sonnei
MTEPLTETPELSAKYAWFFDLDGTLAEIKPHPDQVVVPDNILQGLQLLATASDGALALISGRSMVELDALAKPYRFPLAGVHGAERRDINGKTHIVHLPDAIARDISVQLHTVIAQYPGAELEAKGMAFALHYRQAPQHEDALMTLAQRITQIWPQMALQQGKCVVEIKPRGTSKGEAIAAFMQEAPFIGRTPVFLGDDLTDESGFAVVNRLGGMSVKIGTGATQASWRLAGVPDVWSWLEMISTALQQKRENNRSDDYESFSRSI

SEQ ID NO: 95 - protein, 266 aa, Escherichia coli
MTEPLTETPELSAKYAWFFDLDGTLAEIKPHPDQVVVPDNILQGLQLLATASDGALALISGRSMVELDALAKPYRFPLAGVHGAERRDINGKTHIVHLPDAIARDISVQLHTVIAQYPGAELEAKGMAFALHYRQAPQHEDALMTLAQRITQIWPQMALQQGKCVVEIKPRGTSKGEAIAAFMQEAPFIGRTPVFLGDDLTDESGFAVVNRLGGMSIKIGTGATQASWRLAGVPDVWSWLEMITTALQQKRENNRSDDYESFSRSI

SEQ ID NO: 96 - protein, 266 aa, Shigella flexneri
MTEPLTETPELSAKYAWFFDLDGTLAEIKPHPDQVVVPDNILQGLQLLATASDGALALISGRSMVELDALAKPYRFPLAGVHGAERRDINGKTHIVHLPDAIARDISVQLHTVIAQYPGAELEAKGMAFALHYRQAPQHEDALMTLAQRITQIWPQMALQQGKCVVEIKPRGTSKGEAIAAFMQEAPFIGRTPVFLGDDLTDESGFAVVNRLGGMSVKIGTGATQASWRLAGVPDVWSWLKMITAALQQKRENNRSDDYESFSRSI

SEQ ID NO: 97 - protein, 265 aa, Escherichia albertii
MTEPLTALPELSAKYAWFFDLDGTLAEIKPHPDQVAVPDKILQGLQRLATISHGALALISGRSMVELDALVNPYRFPLAGVHGAERRDINGKTHIVHLPKAMARDICVQLHMAIARFPGAELEEKGMAFALHYRQVPQYEDALLTLARHITQTWPQMVLQQGKCVVEIKPGGTSKGEAIVAFMQETPFIGRKPVFLGDDLTDESGFAVVNRLGGISVKIGTGATQASWRMAGVPDVWRWLEMITHALQKRANDRSDNDEPFSRSI

SEQ ID NO: 98 - protein, 267 aa, Escherichia fergusonii
MTKPLTEAPELSAKYAWFFDLDGTLAEIKPHPDQVAIPDAILQGLQQLAVHSDGALALISGRSMVELDTLAKPYRFPLAGVHGAERRDINGKTHIVRLPDAMVRDISVQLHTTLAGLTGVEIEEKGMAFALHYRQAPQHEALLFTLAQRITQIWPQMALQQGKCVVEIKPRGTCKGDAIAEFMQETPFVGRIPIFWGDDLTDESGFAVVNQAGGISVKIGAGETQAKWRLAGVPDVWRWLAVITNSLQEQKLEENRSDDYESFSRSI

SEQ ID NO: 99 - protein, 191 aa, Escherichia fergusonii
MAKSVPAIFLDRDGTINVDHGYVHEIDNFEFIDGVIDAMRELKKMGFALVVVTNQSGIARGKFTEAQFETLTEWMDWSLADRDVDLDGIYYCPHHPQGSVEEFRQVCDCRKPHPGMLLSARDYLHIDMAASYMVGDKLEDMQAATAANVGTKVLVRTGKPITPEAENAADWVLNSLADLPQAIKKQQKPAQ

SEQ ID NO: 100 - protein, 191 aa, Shigella sonnei
MAKSVPAIFLDRDGTINVDHGYVHEIDNFEFIDGVIDAMRELKKMGFALVVVTNQSGIARGKFTEAQFETLTEWMDWSLADRDVDLDGIYYCPHHPQGSVEEFRQVCDCRKPHPGMLLSARDYLHIDMAASYMVGDKLEDMQAAVAANVGTKVLVRTGKPITPEAENAADWMLNSLADLPQAIKKQQKPAQ

SEQ ID NO: 101 - protein, 191 aa, Escherichia coli
MAKSVPAIFLDRDGTINVDHGYVHEIDNFEFIDGVIDAMRELKKMGFALVVVTNQSGIARGKFTEAQFETLTEWMDWSLADRDVDLDGIYYCPHHPQGSVEEFRQLCDCRKPHPGMLLSARDYLHIDMAASYMVGDKLEDMQAAVAANVGTKVLVRTGKPITPEAENAADWVLNSLADLPQAIKKQQKPAR

SEQ ID NO: 102 - protein, 188 aa, Escherichia albertii
MAKSVPAIFLDRDGTINVDHGYVHEIDNFEFIDGVIDAMRELKKMGFALVVVTNQSGIARGKFTEAQFETLTEWMDWSLADRDVDLDGIYYCPHHPQGSVEEFRQVCDCRKPHPGMLISARDYLHIDMTASYMVGDKLEDMQAASAANVGTKVLVRTGKPVTPEAEDAADWVLNSLADLPQAIKKQQK

SEQ ID NO: 103 - protein, 188 aa, Kluyvera intermedia
MAKSVPAIFLDRDGTINVDHGYVHEIDNFEFIEGVIDAMRELKAMGFALVVVTNQSGIARGKFTEAQFETLTEWMDWSLADRDVDLDGIYYCPHHPQGTVEEFRQVCDCRKPHPGMLISARDYLHIDMSASYMVGDKLEDMQAAAAADVGTKVLVRTGKPVTPEAENAADWVINSLADLPAAIKKQQK

SEQ ID NO: 104 - protein, 188 aa, Citrobacter koseri
MAKSVPAIFLDRDGTINVDHGYVHEIDEFEFIEGVIDAMRELKKMGFALVVVTNQSGIARGKFTEAQFETLTEWMDWSLADRDVDLDGIYYCPHHPQGSVEEFRQACDCRKPHPGMLISARDFLHIDMAASYMVGDKIEDMQAAAAANVGTKVLVRTGKPVTPEAENAADWVLNSLADLPSAIRKQQK

SEQ ID NO: 105 - protein, 188 aa, Kosakonia arachidis
MTKSVPAIFLDRDGTINVDHGYVHEIDNFEFIDGVIDAMRELKEMGYALVLVTNQSGIARGKFTEAQFETLTEWMDWSLADRGVDLDGIYYCPHHPQGTVEEFRQVCDCRKPHPGMLISARDFLHIDMSASYMVGDKIEDMQAAQAAQVGTKVLVRTGKPITPEAEKSADWVINSLAELPLAIKKHQK

SEQ ID NO: 106 - protein, 187 aa, Kluyvera cryocrescens
MAKSVPAIFLDRDGTINVDHGYVHEIDDFEFIDGVIDAMRELKEMGYALVLVTNQSGIARGKFTEAQFETLTEWMDWSLADRGVDLDGIYFCPHHPQGTVEEYRQVCDCRKPHPGMLKSAQEYLHIDMSSSYMVGDKIEDMQAAAAANVGTKVLVRTGKPVTEDAEKAADWVINSLADLPAMIKKQK

SEQ ID NO: 107 - protein, 188 aa, Leclercia adecarboxylata
MAKSVPAIFLDRDGTINVDHGYVHEIDEFEFIEGVIEAMRELKEMGFALVVVTNQSGIARGKFTEAQFETLTEWMDWSLADRGVDLDGIYYCPHHPQGTVEEYRQTCDCRKPHPGMFISAQEFLHIDMSASYMVGDKLEDMQAAAAAGVGHKILVRTGKPVTPEAENAADCVINSLAALPETIKKQQK

SEQ ID NO: 108 - protein, 219 aa, Shigella sonnei
MKLQGVIFDLDGVITDTAHLHFQAWQQIAAEIGISIDAQFNESLKGISRDESLRRILQHGGKEGDFNSQERAQLAYRKNLLYVHSLRELTVNAVLPGIRSLLADLRAQQISVGLASVSLNAPTILAALELRKFFTFCADASQLKNSKPDPEIFLAACAGLGVPPQACIGIEDAQAGIDAINASGMRSVGIGAGLTGAQLLLPSTESLTWPRLSAFWQNV

SEQ ID NO: 109 - protein, 219 aa, Escherichia coli
MKLQGVIFDLDGVITDTAHLHFKAWQQIAAEIGISIDAQFNESLKGISRDESLRRILQHGGKEGDFNPQERAQLAYRKNLLYVHSLRELTVNAVLPGIRNLLADLRAQQIPVGLASVSLNAPTILAALELREFFTFCADASQLKNSKPDPEIFLAACAGLGVPPQACIGIEDAQAGIDAINASGMRSVGIGAGLTGAQLLLPSTDSLTWPRLSAFWQNV

SEQ ID NO: 110 - protein, 219 aa, Escherichia coli
MKLQGVIFDLDGVITDTAHLHFQAWQQIAAEIGIGIDVQFNETLKGISRDESLRRILQHGGKEGDFNVQERAQLAYRKNLLYVHSLRELTVNAVLPGIRPLLADLRAQGIPVGLASVSLNAPTILAALELREFFTFCADASQLKHSKPDPEIFLAACAGLGVPPQACIGIEDAQAGIDAINASGMRSVGIGTSLTGAQLLLPSTESLTWPRLSAFWQNV

SEQ ID NO: 111 - protein, 208 aa, Shigella flexneri
MKLQGVIFDLDGVITDTAHLHFQAWQQIAAEIGISIDAQFNESLKGISRDESLRRILQHGGKEGDFNPQERAQLAYRKNLLYVHSLRELTVNAVLPGIRNLLAELRAQQIPVGLASVSLNAPTILAALELREFFTFCADASQLKNSKPDPEIFLAACAGLGVPPQACIGIEDAQAGIDAINASGMRSVGIGAGLTGAQLLLPSTDSLT

SEQ ID NO: 112 - protein, 216 aa, Shigella sonnei
MRCKGFLFDLDGTLVDSLPAVERAWSNWSRRHGLAPEEVLAFIHGKQAITSLRHFMAGKSEADIAAEFTRLEHIEATETEGITALPGAIALLSHLNKAGIPWAIVTSGSMPVARARHKIAGLPAPEVFVTAERVKRGKPEPDAYLLGAQLLGLAPQECVVVEDAPAGVLSGLAAGCHVIAVNAPADTPGLNEVDLVLHSLEQITVTKQPNGDVIIQ

SEQ ID NO: 113 - protein, 215 aa, Citrobacter werkmanii
  source 1..215: note = NBRC 105721
MQCKGFLFDLDGTLVDSLPAVERAWCNWADRFGLDHAEVLGFIHGKQAITSLRHFMVGKSEAEIAAEFTRLEQIEATETAGITALPGAVDLLNHLNKAGIPWAIVTSGSMPVARARHRVAGLPAPEVFVTAERVKRGKPEPDAYLLGAQLLGLAPQECAVVEDAPAGVLSGLAAGCHVIAVNAPADTPRLDEVDFSLTSLEQISVTKQPNGNVVV

SEQ ID NO: 114 - protein, 215 aa, Citrobacter freundii
MQCKGFLFDLDGTLVDSLPAVERAWCNWADRFGLDHAEVLGFIHGKQAITSLRHFMVGKSEAEIAAEFTRLEQIEATETAGITALPGAVDLLNHLNKAGIPWAIVTSGSMPVARARHRVAGLPAPEVFVTAERVKRGKPEPDAYLLGAQLLGVAPQECAVVEDAPAGVLSGLAAGCHVIAVNAPADTPRLDEVDFSLTSLEHISVTKQPNGNVVV

SEQ ID NO: 115 - protein, 215 aa, Citrobacter amalonaticus
MQCKGFLFDLDGTLVDSLPAVERAWCNWADRFGLAHDDVLSFVHGKQAITSLRHFMAGKPEAEIVAEFTRLEKIEATETAGITALPGAVALLNHLNKAGIPWAIVTSGSMPVARARHQVANLPAPEVFVTAERVKRGKPEPDAYLLGAQLLGLSPHECVVVEDAPAGVLSGLAAGCHVIAVNAPADTPRLDEVDFVLTSLEQLSVTKQPNGDVVV

SEQ ID NO: 116 - protein, 215 aa, Salmonella enterica
VQCKGFLFDLDGTLVDSLPAVERAWCSWADRFNLAHDEVLGFIHGKQAITSLRHFMAGKSEAEIAAEFTRLEQIEATETAGITALPGAVDLLNHLNKAGIPWAIVTSGSMPVARARHQVAGLPAPEVFVTAERVKRGKPEPDAYLLGAQLLGLVPQECVVVEDAPAGVLSGLAAGCHVIAVNAPADTPRLADVDFALNSLTQLSVAKQPNGDVVV

SEQ ID NO: 117 - protein, 216 aa, Escherichia fergusonii
MRCKGFLFDLDGTLVDSLPAVERAWCNWADRFGIDHHELLSFIHGKQAITSLRHFMPGRPEEEILAEFTRLEQIEATQTEGITALPGAIELLTHLNKSGIPWAIVTSGSMPVARARHQVAGLPFPEVFVTAERVKRGKPEPDAYLLGAQLLGLEPKECVVVEDAPAGVLSGLAAGCHVIAVNTPADTPRLSEVDFVLTSLQQITVTKQPNGEVIIQ

SEQ ID NO: 118 - protein, 215 aa, Salmonella enterica
  source 1..215: note = subsp. enterica serovar Hadar
VQCKGFLFDLDGTLVDSLPAVERAWCSWADRFNLAHDEVLGFIHGKQAITSLRHFMAGKSEAEIAAEFTRLEQIEATETAGITALPGAVDLLNHLNKAGIPWAIVTSGSMPVARARHQVAGLPAPEVFVTAERVKRGKPEPDAYLLGAQLLGLSPQECVVVEDAPAGVLSGLAAGCHVIAVNAPADTPRLADVDFALDSLTQLSVAKQPNGDVVV

SEQ ID NO: 119 - protein, 270 aa, Escherichia coli
MAIKLIAIDMDGTLLLPDHTISPAVKNAIAAARARGVNVVLTTGRPYAGVHNYLKELHMEQPGDYCITYNGALVQKAADGSTVAQTALSYDDYRFLEKLSREVGSHFHALDRTTLYTANRDISYYTVHESFVATIPLVFCEAEKMDPNTQFLKVMMIDEPAILDQAIARIPQEVKEKYTVLKSAPYFLEILDKRVNKGTGVKSLADVLGIKPEEIMAIGDQENDIAMIEYAGIGVAMDNAIPSVKEVANFVTKSNLEDGVAFAIEKYVLN

SEQ ID NO: 120 - protein, 270 aa, Escherichia fergusonii
MAIKLIAIDMDGTLLLPDHTISPAVKNAIAAARARGVNVVLTTGRPYAGVHNYLKELHMEQPGDYCITYNGALVQKAADGSTVAQTALSYEDYRFLEKLSREVGSHFHALDRTTLYTANRDISYYTVHESFVATIPLVFCEAEKMDPNTQFLKVMMIDEPTILDQAIARIPQEVKEKYTVLKSAPYFLEILDKRVNKGTGVKSLADVLGIKPEEIMAIGDQENDIAMIEYAGVGVAMDNAIPSVKEVANFVTKSNLEDGVAFAIEKYVLN

SEQ ID NO: 121 - protein, 270 aa, Shigella sonnei
MAIKLIAIDMDGTLLLPDHTISPAVKNAIAAARARGVNVVLTTGRPYAGVHNYLKELHMELPGDYCITYNGALVQKAADGSTVAQTALSYDDYRFLEKLSREVGSHFHALDRTTLYTANRDISYYTVHESFVATIPLVFCEAEKMDPNTQFLKVMMIDEPAILDQAIARIPQEVKEKYTVLKSAPYFLEILDKRVNKSTGVKSLADVLGIKPEEIMAIGDQENDIAMIEYAGVGVAMDNAIPSVKEVANFVTKSNLEDGVAFAIEKYVLN

SEQ ID NO: 122 - protein, 269 aa, Trabulsiella guamensis
MAIKLIAIDMDGTLLLPDHTISPAVKNAIAAARMRGVNVVLTTGRPYAGVHHYLKELHMEQPGDYCITYNGALVQKASDGSTVAQTALSYDDYRFLEKLSREVGSHFHALDRNTLYTANRDISYYTVHESYVATIPLVFCEAEKMDPATQFLKVMMIDEPVVLDKAIARIPAEVKEKYTVLKSAPYFLEILDKRVNKGTGVKSLADVLGIQPDEIMAIGDQENDIAMIEYAGVGVAMDNAIPSVKEVANFITKSNLEDGVAYAIEKYVL

SEQ ID NO: 123 - protein, 270 aa, Citrobacter amalonaticus
MAIKLIAIDMDGTLLLPDHTISPAVKRAIAAAREKGVNVVLTTGRPYAGVHSYLKELHMEQPGDYCITYNGALVQKAGDGSTVAQTALSYDDYRYLEKLSREVGSHFHALDRTTLYTANRDISYYTVHESYVATIPLVFCEAEKMDPKTQFLKVMMIDEPAILDQAIARIPAEVKEKYTVLKSAPYFLEILDKRVNKGTGVKSLADALGIKPEEIMAIGDQENDIAMIEYAGLGVAMDNAIPSVKEIANFVTKSNLEDGVAYAIEQHVLK

SEQ ID NO: 124 - protein, 269 aa, Klebsiella pneumoniae
MAIKLIAIDMDGTLLLPDHTISPAVKTAIAAARERGVNVVLTTGRPYAGVHSYLKELHMEQPGDYCITYNGALVQKAGDGSTVAQTALSYDDYRFLEQLSREVGSHFHALDRNTLYTANRDISYYTVHESYVATIPLVFCEAEKMDPEIQLLKVMMIDEPAILDQAIARIPAEVKEKYTVLKSAPYFLEILDKRVNKGTGVKSLADALGIKPEEIMAIGDQENDIAMIEFAGVGVAMDNAIPAVKEAANFITKSNLEDGVAFAIEKYVL

SEQ ID NO: 125 - protein, 269 aa, Trabulsiella odontotermitis
MAIKLIAIDMDGTLLLPDHTISPAVKNAIAAARMRGVNVVLTTGRPYAGVHHYLKELHMEQPGDYCITYNGALVQKASDGSTVAQTALSYDDYRYLEKLSREVGSHFHALDRNTLYTANRDISYYTVHESYVATIPLVFCEAEKMDPATQFLKVMMIDEPVVLDKAIARIPAEVKEKYTVLKSAPYFLEILDKRVNKGTGVKSLAEALGIQPDEIMAIGDQENDIAMIEYAGVGVAMDNAIPSVKEVANFITKSNLEDGVAYAIEKYVL

SEQ ID NO: 126 - protein, 270 aa, Enterobacter kobei
MAIKLIAIDMDGTLLLPDHTISPAVKKAIAAAREKGVNVVLTTGRPYAGVHNYLRELHMDKPGDYCITYNGALVQKASDGSTVAQTTLSYDDYRYLEQLSREVGSHFHALDRNTLYTANRDISYYTVHESYVATIPLVFCEAEKMDPAIQLLKVMMIDEPAILDKAIARIPAEVKEKYTVLKSAPYFLEILDKRVNKGTGVKSLADTLGITPDEIMTLGDQENDIAMIEYAGLGVAMDNAIDSVKEVADFVTKSNLEDGVAYAIEKFVLN

SEQ ID NO: 127 - protein, 238 aa, Shigella sonnei
MRFYRPLGRISALTFDLDDTLYDNRPVILRTEREALTFVQNYHPALRSFQNEDLQRLRQAVREAEPEIYHDVTRWRFRSIEQAMLDAGLSAEEASAGAHAAMINFAKWRSRIDVPQQTHDTLKQLAKKWPLVAITNGNAQPELFGLGDYFEFVLRAGPHGRSKPFSDMYFLAAEKLNVPIGEILHVGDDLTTDVGGAIRSGMQACWIKPENGDLMQTWDSRLLPHLEISRLASLTSLI

SEQ ID NO: 128 - protein, 223 aa, Shigella sonnei
MRFYRPLGRISALTFDLDDTLYDNRPVILRTEREALTFVQNYHPALRSFQNEDLQRLRQAVREAEPEIYHDVTRWRFRSIEQAMLDAGLSAEEASAGAHAAMINFAKWRSRIDVPQQTHDTLKQLAKKWPLVAITNGNAQPELFGLGDYFEFVLRAGPHGRSKPFSDMYFLAAEKLNVPIGEILHVGDDLTTDVGGAIRSGMQACWIRPENGDLMQTWDSRLL

SEQ ID NO: 129 - protein, 238 aa, Salmonella enterica
MRFYRPLGRIAALTFDLDDTLYDNRPVILRTEQEALAFMQNYHPSLRSFQNVDLQRIRQAVREAEPEIYHDVTRWRHRAIEQAMRDAGLSAQEAIAGANAAMMHFAKWRSQIEVPQATHETLQQLAKKWPLVAITNGNAQPELFGLGDYFKFVLRAGPDGRSKPFSDMYFLVAEKLHVPIGEILHVGDDLTTDVAGAIRCGMQACWIKPENADLMRTQDSRLLPHIEISRLASLTSLI

SEQ ID NO: 130 - protein, 238 aa, Citrobacter braakii
MRFYRPLGRISALTFDLDDTLYDNRPVITRTEQEALAFVQNYHPALNSLQNSDLQRLRQAVRDAEPEIYHDVTQWRHRAVERAMLEAGLSEAEAKMGANAAMMNFAKWRSRIDVPQSTHDTLKTLARKWPLVAITNGNAQPELFGLGDYFEFVLRAGPDGRSKPFSDMYALAAEKLKMPVGEILHVGDDLTTDVAGAIRCGMQACWIKPENADLMQTADSRLLPHIEISQLASLTSLI

SEQ ID NO: 131 - protein, 238 aa, Enterobacter hormaechei
MRFYRPLGQISALTFDLDDTLYDNRPVILRTEQESLAFVQNYHPALKTMQNKDFQKLRQSLRETEPDIYHDVTEWRRRAVEQAMLNVGLSSQDAAIGAEAAMENFAKWRSRVDVPQETHDTLAKLAEKWPLVAITNGNAQPELFGLGDYFEFVLRAGPHGRSKPFSDMYHLAAEKLNLPLGEILHVGDDLTTDVAGAIRCGMQACWIKPENASLMTTPDSRLLPHLEISRLASLTTLI

SEQ ID NO: 132 - protein, 238 aa, Lelliottia amnigena
MRFYRPLGQISALTFDLDDTLYDNRQVILRTEQEALAFVQNYHPSLKTLQNTDFQRLRQALRETEPDIYHDVTEWRRRAVEQAMINAGLTAAEAALGAEASMANFAKWRSRIDVPQETHDTLAKLAEKWPLVAITNGNAQPELFGLGDYFTFVFRAGPHGRSKPFSDMYHLAAEKLDLPLGEILHVGGDLTTDVAGAIRCGMQACWIKPENADLMHTIDSRLLPHVEISRLASLTTLI

SEQ ID NO: 133 - protein, 238 aa, Leclercia adecarboxylata
MRFYRPLGQISALTFDLDDTLYDNRQVILRTEQEALTFVQNYHPALKTLENKEFHRLRQALRQTEPEIYHDVTEWRRRAVELAMLNAGLTAAEAALGAEASMAHFAQWRSRIDVPQETHDTLAALAEKWPLVAITNGNAQPELFGLGDYFQFVLRAGPHGRSKPFNDMYHLAAEKLSLPLGQILHVGDDLTTDVAGAIRCGMQACWIKPENADLMQTADSRLLPHIEISRLASLTTLI

SEQ ID NO: 134 - protein, 190 aa, Shigella flexneri
MSTPRQILAAIFDMDGLLIDSEPLWDRAELDVMASLGVDISRRNELPDTLGLRIDMVVDLWYARQPWNGPSRQEVVERVIARAISLVEETRPLLPGVREAVALCKEQGLLVGLASASPLHMLEKVLTMFDLRDSFDALASAEKLPYSKPHPQVYLDCAAKLGVDPLTCVALEDSVNGMIASKAARMRSIV

SEQ ID NO: 135 - protein, 222 aa, Kosakonia sacchari
MTTPRQILAAIFDMDGLLIDSEPLWDRAELEVVASLGVDITRRHELPDTLGLRIDMVVELWYAQQPWNGPDRQEVTECIIQRAISLVEEKRPLLPGVREAIALCKANGLLVGLASASPLHMLEKVLAMFELRDSFDALASAEKLPYSKPHPQVYLDCAAKLGVDPLTCVALEDSVNGMIASKAARMRSIVVPDEEHRADPRYVLANVKLTSLEQLTLAHPIG

SEQ ID NO: 136 - protein, 222 aa, Enterobacter mori
MSTPRQILAAIFDMDGLLIDSEPLWDRAELDVMASLGVDISRRNELPDTLGLRIDMVVDLWYAHQPWVGPGRDEVIARIINRAITLVEEQKPLLPGVRDAIALCKAQGLKVGLASASPLHMLEKVLSLFELRDSFDALASAEKLPYSKPHPQVYMDCAAKLGLDPLTCVALEDSVNGMVASKAARMRSIVVPAEEGRHDPRFALADVKLASLEDLTVAHLRG

SEQ ID NO: 137 - protein, 222 aa, Lelliottia amnigena
MSTSRQIVAAIFDMDGLLIDSEPLWDRAELDVMESLGVDIRRRNELPDTLGLRIDMVVELWYAHQPWNGPSRQEVTDRIISRALTLVEASRPLLPGVREAVALCKAQGLKVGLASASPLHMLEKVLAMFDLRESFDALASAEKLPYSKPHPQVYMDCAAKLGVDTLACVALEDSVNGMIASKAARMRSVVVPAEEGQHDPRFALADVKLATLADLTPAHLRG

SEQ ID NO: 138 - protein, 222 aa, Enterobacter sp.
  source 1..222: note = 638
MSTPHQIVAAIFDMDGLLIDSEPLWDRAELDVMASLGVDISRRGELPDTLGLRIDMVVELWFAHQPWSGPSREEVTARVISRAIALVEEKRPLLPGAREAIALCKAQGLKVGLASASPLHMLEKVLEMFDLRDSFDALASAEKLPYSKPHPQVYMDCAAKLGVDPLACVALEDSVNGMVASKAARMRSIVVPAEEGQHDPRFALANAKLTSLVDLTPAHLFG

SEQ ID NO: 139 - protein, 222 aa, Kosakonia radicincitans
MTAPRQILAAIFDMDGLLIDSEPLWDRAELEVVASLGVDINRRHELPDTLGLRIDMVVELWYAQQPWNGPDRKEVTERIIQRAISLVEEKRPLLPGVREAIALCKANGLLVGLASASPLHMLEKVLAMFELRDSFDALASAENLPYSKPHPQVYLDCAAKLGIDPLTCVALEDSVNGMIASKAARMRSIVVPDEEHRTDPRYVLANVKLTSLEQLTLAHLIG

SEQ ID NO: 140 - protein, 209 aa, Salmonella enterica
  source 1..209: note = subsp. enterica serovar Newport str.
MDGLLIDSEPLWDRAELDVMASLGVDITRRHELPDTLGLRIDMVVDLWFAQQPWNGPDRQEVTNRVIARAITLIEETRPLLPGVREAVALCKAQGLLVGLASASPLHMLEKVLTMFELRDSFDALASAEKLPYSKPHPQVYLDCAAKLGVDPLTCVALEDSVNGLIAAKAARMRAIVVPAEENQHDPRFALANVKLNSLTELTAAHLLG

SEQ ID NO: 141 - protein, 188 aa, Shigella flexneri
MYERYAGLIFDMDGTILDTEPTHRKAWREVLGHYGLQYDVQAMIALNGSPTWRIAQAIIELNQADLDPHALAREKTEAVRSMLLDSVEPLPLVEVVKSWHGRRPMAVGTGSESAIAEALLAHLGLRRYFDAVVAADHVKHHKPAPDTFLLCAQRMGVQPTQCVVFEDADFGIQAARAAGMDAVDIRLL

SEQ ID NO: 142 - protein, 188 aa, Escherichia albertii
MYERYAGLIFDMDGTILDTEPTHRKAWREVLGRYGLRYNVQAMIALNGSPTWRIAQAIIELNQADLDPYALAREKTEAVRSMLLDSVEPLPLVEVVKSWYGRRPMAVGTGSESAIAEALLTHLGLRRYFDTVVAADHVKHHKPAPDTFLLCAQHMGVQPAQCVVFEDADFGIQAARAAGMDAVDVRLL

SEQ ID NO: 143 - protein, 188 aa, Salmonella enterica
MYARYAGLIFDMDGTLLDTEPTHRKAWREVLGHYGLRFDEQAMVALNGSPTWLIAQSIIELNHADLDPLSLAREKTDAVKSILLDCVEPLPLVEVVKAWHGRRPMSVGTGSESAIAEALLAHLGLRRYFDAVVAADHVQHHKPAPDTFLLCAQRMGVMPTQCVVFEDADFGLQAARAAGMDAVDVRLL

SEQ ID NO: 144 - protein, 188 aa, Kluyvera intermedia
MYERYDGLIFDMDGTILDTEPTHRKAWHDVLGHYGLRYDIQAMIALNGSPTWRIAQSVIELNQADLDPYALAREKTEAVKAMLLDTVRPLPLIDVVKAWYGRRPLSVGTGSESAIAEALLSHLGLRHYFAAVVAAEHVKNHKPAPDTFLLCAEKMGVAPQKCVVFEDADFGLQAARSAGMDAVDVRLL

SEQ ID NO: 145 - protein, 188 aa, Trabulsiella odontotermitis
MYERYAGLIFDMDGTILDTEPTHRKAWHEVLGRYGIRFDEQSIIALNGSPTWRIAQEIITLNQADLDPHALAREKTDAVKIMLLDSVQPLPLIDVVKAWHGRRPMSVGTGSESAIAEALLAHLGLRHYFSAVVAADHVRHHKPAPDTFLLCAERMGVEPAKCIVFEDADFGLQAAASAGMDVVDVRLL

SEQ ID NO: 146 - protein, 188 aa, Yokenella regensburgei
MYERYAGLIFDMDGTILDTEPTHCKAWHDVLGRYGMHFDEQAMTALNGSPTWRIAQSIIELNQATLDPHQLAREKTDAVKAMLLDTVRPLPLIDVVKAWHGRRPMAVGTGSESAIAEALLAHLGLREYFTAVVAADHVKHHKPAPDTFLLCAELMGVAANQCVVFEDADFGLQAARSAGMDVVDVRLL

SEQ ID NO: 147 - protein, 188 aa, Raoultella terrigena
MYERYAGLIFDMDGTILDTEPTHRKAWHEVLGRYGMRFDEQAMVALNGSPTWRIAQVIIELNQADLDPHRLALEKTNAVKAMLLDSVRPLPLIEVVKEWHGRRPMSVGTGSESAVAEALLAHLGLRHYFSAVIAADHVTNHKPAPDTFLLCAERMGVAPEKCVVFEDADFGLQAARRAGMDAVDVRLL

SEQ ID NO: 148 - protein, 188 aa, Klebsiella pneumoniae
MYEGYAGLIFDMDGTILDTEPTHRQAWNEVLGRYGMRFDEQAMVALNGSPTWRIAQAIIELNQADLDPHRLAQEKTQAVKAMLLDSVRPLPLIEVVKAWHGCRPMSVGTGSESAVAEALLAHLGLRHYFSAVVAADHVVNHKPAPDTFLLCAERMGVAPEKCVVFEDADFGLQAAKRAGMDAVDVRLL

SEQ ID NO: 149 - protein, 223 aa, Pseudomonas putida
MRLRAVLFDMDGTLLDTAPDFIAICQAMLADRGLPAIDDARIREVISGGARAMVAATFAMNPEADGFEALRLEFLERYQRDCAVHSKLFDGMPELLADIEKGNLLWGVVTNKPVRFAEPIMQRLGLAERSALLICPDHVKNSKPDPEPLILACKTLGLDPASVLFVGDDLRDIESGRDAGTRTAAVRYGYIHPEDNPNNWGADVVVDHPLELRKVIDSALCGC

SEQ ID NO: 150 - protein, 223 aa, Pseudomonas sp.
  source 1..223: note = GM84
MRLRAVLFDMDGTLLDTAPDFIAICQAMLAERGLPAIDDKLIRDVISGGARAMVAATFAMNPDDDGFEPLRLEFLERYQRDCAVHSKLFDGMGELLADIEKGNLLWGVVTNKPVRFAEPIMQRLGLAERSALLICPDHVKNSKPDPEPLILACKTLNLDPASVLFVGDDLRDIESGRDAGTRTAAVRYGYIHPEDNPNNWGADVVVDHPLELRKVIDSALCGC

SEQ ID NO: 151 - protein, 223 aa, Pseudomonas entomophila
MRLRAVLFDMDGTLLDTAPDFIAICQAMLTDRGLPTIDDARIRDVISGGARAMVAATFAMDPDAEGFEALRLEFLERYQRDCAVHSKLFDGMPELLADIEKGNLLWGVVTNKPVRFAEPIMQRLGLAERSALLICPDHVKNSKPDPEPLILACKTLDLDPASVLFVGDDLRDIESGRDAGTRTAAVRYGYIHPEDNPNNWGADVVVDHPLELRKVIDSALCGC

SEQ ID NO: 152 - protein, 223 aa, Pseudomonas vranovensis
MRLRAVLFDMDGTLLDTAPDFIAICQAMLADRGLPAIDDQRIRDVISGGARAMVEVTFGITPEVAEFEALRLEFLERYQRDCAVHSKLFDGMAELLADIEKGNLLWGVVTNKPVRFAEPIMQRLGLAERSALLICPDHVKNSKPDPEPLILACKTLNLDPASVLFVGDDLRDIESGRDAGTRTAAVRYGYIHPEDNPNNWGADVVVDHPLELRKVLDSALCGC

SEQ ID NO: 153 - protein, 223 aa, Pseudomonas cannabina
MRLRAVLFDMDGTLLDTAPDFIAIAQAMLADRGLPAVADKLIRDEISGGAKAMVAAAFALSPEAPEFEALRLEFLERYQRDCATHSRLFNGMPELLADIEKAGLIWGVVTNKPVRFAQPIMEQLKLAERSAVLICPDHVTHSKPHPEPMILACKLLDLDPASVLFVGDDLRDIESGRDAGTKTAAVRYGYIHPDDNPDHWGADVVVDHPLELRKVLDNALCSC

SEQ ID NO: 154 - protein, 223 aa, Pseudomonas monteilii
MRLRAVLFDMDGTLLDTAPDFIAICQAMLAERGLPAIDDQAIRDVISGGARAMVAATFAMNPEDEGFEALRLEFLERYQRDCAVHSKLFDGMGELLADLEKGKLLWGVVTNKPVRFAEPIMQQLGLAERSALLICPDHVKNSKPDPEPLILACKTLNLDPASVLFVGDDLRDIESGRDAGTRTAAVRYGYIHPDDNPNHWGADVVVDHPLELRKVIDSALCGC

(19) United States
(12) Patent Application Publication
(10) Pub. No.: US 2026/0132407 A1
(43) Pub. Date: May 14, 2026

(54) VIVO SYNTHESIS OF SIALYLATED COMPOUNDS

(71) Applicant: Inbiose N.V., Zwijnaarde (BE)

(72) Inventors: Joeri Beauprez, Zwijnaarde (BE); Pieter Coussement, Zwijnaarde (BE); Dries Van Herpe, Zwijnaarde (BE); Gert Peters, Zwijnaarde (BE); Annelies Vercauteren, Zwijnaarde (BE)

(21) Appl. No.: 19/259,734

(22) Filed: Jul. 3, 2025

(30) Foreign Application Priority Data
Dec. 27, 2016 (EP) 16206916.5

(51) Int. Cl. C12N 15/52; C12N 9/10; C12N 9/90

(52) U.S. Cl. CPC C12N 15/52; C12N 9/1022; C12N 9/1051; C12N 9/1081; C12N 9/1085; C12N 9/90; C12Y 204/01038; C12Y 204/0109; C12Y 205/01056

(57) ABSTRACT

This disclosure is in the technical field of synthetic biology and metabolic engineering. More particularly, the disclosure is in the technical field of fermentation of metabolically engineered microorganisms. The disclosure describes engineered microorganisms able to synthesize sialylated compounds via an intracellular biosynthesis route. These microorganisms can dephosphorylate N-acetylglucosamine-6-phopshate to N-acetyl glucosamine and convert the N-acetylglucosamine to N-acetylmannosamine. These microorganisms also have the ability to convert N-acetylmannosamine to N-acetyl-neuraminate. Furthermore, provided is a method for the large scale in vivo synthesis of sialylated compounds, by culturing a microorganism in a culture medium, optionally comprising an exogenous precursor such as, but not limited to lactose, lactoNbiose, N-acetyllactosamine and/or an aglycon, wherein the microorganism intracellularly dephosphorylates N-acetylglucosamine-6-phopshate to N-acetylglucosamine, converts N-acetylglucosamine to N-acetylmannosamine and convert the latter further to N-acetyl-neuraminate.

### CROSS REFERENCE TO RELATED APPLICATIONS

This application is a divisional of U.S. patent application Ser. No. 17/993,441, filed Nov. 23, 2022, which is a continuation of U.S. patent application Ser. No. 16/473,932, filed Jun. 26, 2019, now U.S. Pat. No. 11,535,878, which is a 371 of PCT/EP2017/084593 Dec. 26, 2017, which claims the benefit of European Patent Office (EPO) application Ser. No. 16/206,916.5 filed Dec. 27, 2016. the disclosure of each of which is hereby incorporated herein in its entirety by this reference.

### STATEMENT REGARDING ELECTRONIC FILING OF A SEQUENCE LISTING

Pursuant to 37 C.F.R. § 1.831 through 1.835, a Sequence Listing XML file entitled “6ZH5700-004-PCT Sequence Listing ST26 (updated)” 176 KB in size, generated Jul. 1, 2025, has been submitted via EFS-Web is provided in lieu of a paper copy. This Sequence Listing is hereby incorporated by reference into the specification for its disclosures.

### TECHNICAL FIELD

This disclosure relates generally to synthetic biology and metabolic engineering. More particularly, the disclosure relates to fermentation of metabolically engineered microorganisms. Described are engineered microorganisms able to synthesize sialylated compounds via an intracellular biosynthesis route. These microorganisms can dephosphorylate N-acetylglucosamine-6-phosphate to N-acetyl glucosamine and convert the N-acetylglucosamine to N-acetylmannosamine. These microorganisms also have the ability to convert N-acetylmannosamine to N-acetyl-neuraminate. Furthermore, provided is a method for the large scale in vivo synthesis of sialylated compounds, by culturing a microorganism in a culture medium, optionally comprising an exogenous precursor such as, but not limited to lactose, lacto-N-biose, N-acetyllactosamine and/or an aglycon, wherein the microorganism intracellularly dephosphorylates N-acetylglucosamine-6-phosphate to N-acetylglucosamine, converts N-acetylglucosamine to N-acetylmannosamine and convert the latter further to N-acetyl-neuraminate.

### BACKGROUND

Sialylated compounds such as sialic acid and sialylated oligosaccharides have gained attention over the last years, because of their broad application range. For example, sialic acid is considered as an anti-viral precursor. Sialylated oligosaccharides form an essential part of human milk and are ascribed anti-adhesive and immunomodulatory properties; others described them to be involved in brain development. Sialylation, in general, of proteins, lipids or aglycons are used in anti-cancer medicine and in the treatment of neurological diseases.

Sialic acid is a general term used to describe a large family of acidic sugars that are predominantly found on the cell surface of eukaryotic cells. The most common sialic acid is N-acetylneuraminic acid (“Neu5Ac”), an acidic nine-carbon sugar that undergoes several modifications to generate the members of the sialic acid family. As seen in e.g., FIG. 1 of WO2008097366, the diversity of the sialic acid family is represented with over 50 known members. Sialic acid represents a large family of cell-surface carbohydrates that are derived from an acidic, nine-carbon parent compound called Neu5Ac. Neu5Ac is often decorated with acetyl, phosphate, methyl, sulfate and lactyl groups, which are described to be required for desirable cell signaling and cell adhesion events mediated by sialic acid.

Sialic acids and sialylated compounds are common in higher eukaryotic organisms, which produce them in a conserved biosynthetic route. This route starts from endogenic UDP-N-acetylglucosamine, which is converted to sialic acid through the action of a UDP-N-acetylglucosamine 2-epimerase (hydrolyzing) (EC 3.2.1.183), a N-acylmannosamine kinase (EC 2.7.1.60), a N-acylneuraminate-9-phosphate synthase (EC 2.5.1.57) and a Neu5Ac-9-P phosphatase (EC 3.1.3.29). This sialic acid can subsequently be activated and transferred to the desired acceptor via a CMP-sialic acid synthase (EC 2.7.7.43) and e.g., a sialyltransferase.

Efforts have been made to express this biosynthetic route in other eukaryotic organisms, whereas prokaryotic systems were not reported. The pathway was functionally expressed in yeast (Pichia pastoris) and plant (Arabidopsis thaliana) to produce sialylated N-glycans. However, large scale production of sialylated oligosaccharides was never reported. The functional overexpression of eukaryotic genes in prokaryotic systems remains a daunting task without certain outcome due to the lack of specific chaperones, faulty enzyme folding and missing cell organelles. On top of that remains the huge energy requirement of the pathway and the depletion of intercellular UDP-GlcNAc (UDP-N-acetylglucosamine), necessary for cell growth.

Processes based on enzymatic, chemical as well as fermentative production of sialylated compounds exist. However, all of them have significant disadvantages. For instance, chemical synthesis requires many sequential chemical steps and enzymatic synthesis requires expensive precursors, whereas the fermentative process is still under heavy development. Nonetheless, the latter has the highest industrial production potential.

One type of described fermentative production process uses a biosynthesis route that originates from prokaryotes like Campylobacter jejuni that naturally produces sialic acid or sialylated compounds. This biosynthesis route starts from endogenous UDP-N-acetylglucosamine, which cells use for their cell wall. This is converted to N-acetylmannosamine and N-acetylneuraminate by the action of an UDP-N-acetylglucosamine epimerase (generally named neuC) and a sialic acid synthase (generally named neuB).

Using only part of this prokaryotic biosynthesis route, Priem et al. (Glycobiology 12, 2002, 235-240) describe the use of living bacterial cells to produce sialyloligosaccharides. In this method, sialyllactose was directly produced by growing cells of metabolically engineered Escherichia coli strains, which overexpressed the Neisseria meningitidis genes for α-2,3-sialyltransferase and for CMP-Neu5Ac synthase, these strains were further devoid of β-galactosidase and N-acetylneuraminic acid aldolase activities. These microorganisms were grown at high cell density with glycerol as the carbon and energy source, while exogenous lactose and Neu5Ac were supplied as precursors for sialyllactose synthesis. During the growth, lactose and Neu5Ac were internalized by the induction of the expression of an E. coli galactoside and an exogenous Neu5Ac permease. Lactose and Neu5Ac accumulate in the cytoplasm where Neu5Ac was then converted into CMP-Neu5Ac to be further transferred on lactose to form sialyllactose. Large scale production of sialyloligosaccharides by this microbiological method requires important amounts of Neu5Ac as a precursor.

Another microbial system was developed for producing sialyloligosaccharides without the need of an exogenous supply of sialic acid. WO2007101862 describes a method for producing sialylated oligosaccharides with microorganisms having heterologous genes encoding a CMP-Neu5Ac synthetase, a sialic acid synthase, an UDP-GlcNAc-6-phosphate 2-epimerase and a sialyltransferase, and wherein the endogenous genes coding for sialic acid aldolase (NanA) and for ManNAc kinase (NanK) have been deleted or inactivated. The use of this prokaryotic biosynthesis route is very energy intensive for the cell. Furthermore, the described route for producing the sialylated oligosaccharides competes for the UDP-GlcNAc, which is essential for the cells own peptidoglycan synthesis. Building on this concept, Kang et al. created a production host that does not use a sialic acid synthase, but the endogenous sialic acid aldolase, which has a less favorable chemical equilibrium (Metabolic engineering 14, 2012, 623-629). EP1484406 describes production of Neu5Ac using E. coli overexpressing N-acetylglucosamine 2-epimerase and Neu5Ac synthase, but needs N-acetylglucosamine (GlcNAc) as external precursor. In that method, GlcNAc needs to be used as such. Therefore, the cells in EP1484406 need to be disrupted such that the GlcNAc can be used directly by the GlcNAc-2-epimerase. As described by Lundgren et al. (Org. Biomol. Chem., 2007, 5, 1903-1909) intact cells convert the incoming GlcNAc to N-acetylglucosamine-6-phosphate (GlcNAc-6-P) which is used by the cell for cell growth. This GlcNAc-6-P is unavailable intercellularly and cannot therefore be used for the GlcNAc-2-epimerase, which needs a non-phosphorylated GlcNAc for epimerization to ManNAc. This explains why permeabilization of the cells of EP1484406 is necessary. As explained by Lundgren et al. the GlcNAc-6-P can be used for making Neu5Ac but this requires another synthesis pathway comprising UDP-GlcNAc as an intermediate, which is described in WO2007101862. The resulting pathway further increases energy demand compared to the one described in the latter patent because uridylation of GlcNAc requires an extra ATP.

Deng et al. (Metabolic Engineering 7 (2005), 201-214) describes the production of GlcNAc via intracellular production of GlcNAc-6-P, which is then efficiently dephosphorylated and secreted into the medium as GlcNAc. According to Deng et al. this dephosphorylation happens upon export, more specifically in the periplasm of E. coli. The extracellular produced GlcNAc described in this method, is not available for intracellular conversion. This method to produce GlcNAc requires a two-phase fed batch process, i.e., a cell growth phase followed by a GlcNAc production phase, which is only induced after the culture had reached a high cell density, to minimize inhibitory effects of phosphorylated amino sugars.

Others have attempted the same by heterologously expressing phosphatases and encountered the problem of reduced growth and strong metabolic burden (Lee and Oh, Metabolic engineering, 2015, 143-150). The main reason for the reduction in growth/biomass formation is the non-specificity of the phosphatase that is introduced, which dephosphorylates other essential phosphorylated compounds. Such modifications hence lead to reduced fitness and lower specific productivity. It furthermore leads to selective pressure to mutate the production pathway during production, which reduces the overall process stability.

The production pathways of sialic acid and sialylated oligosaccharides require the formation of high level of phosphorylated (e.g., GlcNAc-6-P) and nucleotide pathway intermediates. It is commonly understood that such formation leads to aspecific degradation of these intermediates by activation of aspecific phosphatases, which in turn leads to reduced fitness. In order to circumvent the effect of the expression of metabolic pathways on the growth of the production hosts, it is standard to use inducible expression systems. In this method first biomass is formed and later in the production process the production pathway is activated by, for instance, IPTG. This was applied by others for the production of sialic acid and sialylated oligosaccharides (WO2007101862; Priem et al. Glycobiology 12, 2002, 235-240; Kang et al. Metabolic engineering 14, 2012, 623-629; Yang et al. Metabolic engineering 43, 2017, 21-28). Apart from losing productivity and titer, another downside in the use of inducible systems is the excretion of intermediate pathway metabolites such as GlcNAc and ManNAc. This leads to the requirement of extra downstream processing steps for the purification, hence a higher production cost in the production of sialic acid, sialyllactose or other sialylated compounds.

The methods for producing sialylated compounds, discussed hereabove, are still insufficient in meeting the large demand of the biotechnological, pharmaceutical, and medical industries. A metabolic engineering approach that successfully overcomes the problems referred to above, would represent a significant and long-awaited advance in the field.

### BRIEF SUMMARY

Surprisingly, described is a production pathway that does not require induction, and does not require a UDP-GlcNAc epimerase, but allows constitutive expression, which also allows better tuning of the metabolic pathway improving production and reducing byproduct formation during the production process.

According to one embodiment, provided is a method for producing sialylated compounds with microorganisms, which method does not require induction.

According to a further embodiment, there is provided a production pathway that does not require a UDP-GlcNAc epimerase, and comprising modulating expression of phosphatase, which does not pose a metabolic burden to the cell as was shown previously in the art. The further embodiment of provided is also an increased sialylated compound production by modulating the expression of phosphatase.

In another further embodiment, the above method, when combined with the constitutive expression of the genes of the metabolic pathway, also allows better tuning of the metabolic pathway reducing byproduct formation during the production process. The disclosure describes an economical, more efficient and alternative biosynthesis route for the production of sialylated compounds using microorganisms.

Provided is a method of producing sialylated compounds by fermentative growth of microorganisms. In particular, the disclosure relates to a method for producing sialylated compounds, wherein the method comprises culturing a microorganism in a culture medium. The microorganism intracellularly converts following reactions: N-acetylglucosamine-6-phosphate to N-acetylglucosamine, N-acetylglucosamine to N-acetylmannosamine, and N-acetylmannosamine to N-acetyl-neuraminate. Furthermore, this microorganism is unable to: i) convert N-acetylglucosamine-6-P to glucosamine-6-P, ii) convert N-acetylglucosamine to N-acetylglucosamine-6-P, and iii) convert N-acetyl-neuraminate to N-acetyl-mannosamine.

Preferably, the conversion of N-acetylglucosamine-6-phosphate to N-acetylglucosamine is obtained by the action of an intracellularly expressed phosphatase. In another preferred embodiment the N-acetylglucosamine is converted to N-acetylmannosamine by an intracellularly expressed N-acetylmannosamine epimerase. In an alternative preferred embodiment the N-acetylmannosamine is converted by an intracellular expressed sialic acid synthase to N-acetyl-neuraminate. Even more preferably, the microorganism comprises all three enzymes such that the microorganism converts i) N-acetylglucosamine-6-phosphate to N-acetylglucosamine by action of an intracellularly expressed phosphatase, ii) the N-acetylglucosamine to N-acetylmannosamine by an intracellularly expressed N-acetylmannosamine epimerase; and iii) the N-acetylmannosamine to N-acetyl-neuraminate by an intracellular expressed sialic acid synthase.

Preferably, the microorganism used in the method of the disclosure is unable to produce following enzymes i) a N-acetylglycosamine-6-phosphate deacetylase, ii) a N-acetylglucosamine kinase, and iii) a N-acetylneuraminate aldolase.

The disclosure also provides a microorganism that expresses i) a phosphatase to dephosphorylate N-acetylglucosamine-6-phosphate to N-acetylglucosamine (EC 3.1.3.), ii) a GlcNAc 2-epimerase to convert N-acetylglucosamine (GlcNAc) to N-acetylmannosamine (manNac) (EC 5.1.3.8), and iii) a sialic acid synthetase to synthesize N-acetyl-neuraminate (Neu5Ac) from N-acetylmannosamine (ManNAc) (EC 2.5.1.56). Furthermore, this microorganism is unable to: i) convert N-acetylglucosamine-6-P to glucosamine-6-P, ii) convert N-acetyl-glucosamine to N-acetyl-glucosamine-6-P, and iii) convert N-acetyl-neuraminate to N-acetyl-mannosamine.

In one aspect, provided is a microorganism that is enabled to catalyze the following reactions: the intracellular conversion of N-acetylglucosamine-6-phosphate to N-acetylglucosamine, the intracellular conversion of N-acetylglucosamine to N-acetylmannosamine and, the intracellular conversion of N-acetylmannosamine to sialic acid.

It is generally accepted that N-acetylglucosamine-6-phosphate is naturally efficiently excreted out of the cell and meanwhile dephosphorylated by phosphatases in the periplasm (see p. 212, second column, Deng et al. Metabolic Engineering 7 (2005), 201-214). Therefore, without the disclosure, this excreted product would be unavailable for conversion to sialic acid. Furthermore, re-internalization occurs through transport proteins, which phosphorylate the N-acetylglucosamine.

The use of an intracellular N-acetylglucosamine-2-epimerase ensures lower energy (ATP) consumption than the classical prokaryotic route (via UDP-N-acetylglucosamine). This enables a more efficient production of sialic acid, sialylated oligosaccharides and/or sialylated products with a healthier and more efficient strain. By optimizing expression levels, the unfavorable chemical equilibrium is overcome and no need of large amounts of free N-acetylglucosamine are necessary, as is in literature. Indeed, in the art, this enzyme is solely used in enzymatic reactions, which use high concentrations of N-acetylglucosamine to produce N-acetylmannosamine. It would be hence logical that the use of an epimerase would require large amounts of intracellular formed GlcNAc, which is shown to be released in the medium (see Deng as described supra), however, the disclosure has proven this can be avoided. Another advantage of the disclosure over enzymatic methods, is that inexpensive substrates can be used in the disclosure, as, e.g., a monosaccharide such as, e.g., glucose, galactose or fructose, a disaccharide such as, e.g., sucrose or maltose or a polyol, such as, but not limited to, glycerol. This enables an economic production method by fermentation.

Different phosphatases (EC 3.1.3.) that convert N-acetylglucosamine-6-phosphate into N-acetylglucosamine are described in the art and can be used in the disclosure. Phosphatases from the HAD superfamily and the HAD-like family are described in the art. Examples from these families can be found in the enzymes expressed from genes yqaB, inhX, yniC, ybiV, yidA, ybjI, yigL or cof from E. coli. One phosphatase that catalyzes this reaction is identified in Blastocladiella emersonii. Phosphatases are generally aspecific and the activity is generally not related to the family or structure. Other examples can thus be found in all phosphatase families. Specific phosphatases are easily identified and screened by well-known methods as described by Fahs et al. (ACS Chem. Biol., 2016, 11 (11), 2944-2961).

Preferably, the phosphatase of the disclosure is a HAD-like phosphatase. A HAD-like phosphatase as defined herein refers to any phosphatase polypeptide, which comprises:
any one or more of the following motifs as defined below:

Motif 1:
(SEQ ID NO: 73)
hDxDx[TV],
or
Motif 2:
(SEQ ID NOs: 74, 75, 76, 77)
[GSTDE][DSEN]x(1-2)[hP]x(1-2)[DGTS]

wherein h means a hydrophobic amino acid (A, I, L, M, F, V, P, G) and x can be any distinct amino acid.

In another preferred embodiment, HAD-like polypeptides typically have in increasing order of preference at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to any one of the polypeptides represented by SEQ ID NOs: 43, 44, 45, 47, 48, 50, 51, 52, 54, 55 or 57. Preferably, those polypeptides also comprise at least one of the above identified Motifs. More preferably, they comprise both motifs.

The overall sequence identity may be determined using a global alignment algorithm, such as the Needleman Wunsch algorithm in the program GAP (GCG Wisconsin Package, Accelrys), preferably with default parameters and preferably with sequences of mature proteins (i.e., without taking into account secretion signals or transit peptides). Compared to overall sequence identity, the sequence identity will generally be higher when only conserved domains or motifs are considered.

In a preferred embodiment, the HAD-like polypeptide comprises any one of SEQ ID NOs: 43, 44, 45, 47, 48, 50, 51, 52, 54, 55, or 57.

In another preferred embodiment, the phosphatase is chosen from the HAD superfamily or the HAD-like phosphatase family. More preferably, the phosphatase is selected from the group comprising: i) enzymes expressed by the genes yqaB, inhX, yniC, ybiV, yidA, ybjI, yigL or cof from E. coli, ii) the phosphatase of B. emersonii and iii) other phosphatase families.

Examples of N-acetyl-D-glucosmine-2-epimerase (EC 5.1.3.8) can be found in prokaryotes and eukaryotes. Examples for prokaryotes are found in cyanobacteria like, e.g., Acaryochloris marina, Anabaena variabilis, Anabaena marina, Nostoc punctiforme, Acaryochloris species, Anabaena species, Nostoc species and Synechocystis species. They are also found in Bacteroides species like, e.g., Bacteroides ovatus and Bacteroides thetaiotaomicron and in Capnocytophaga canimorsus and Mobiluncus mulieris. In eukaryotes, N-acetyl-D-glucosmine-2-epimerase is found in Glycin max, Mus musculus, Homo sapiens, Rattus norvegicus, Bos taurus, Sus scrofa, Canis lupus. Preferably, in the method and microorganism of the disclosure, N-acetylmannosamine-2-epimerase is selected from the group consisting of i) N-acetylmannosamine-2-epimerase from cyanobacteria, more, in particular, from A. marina, A. variabilis, Anabaena marina, Nostoc punctiforme, Acaryochloris species, Anabaena species, Nostoc species and Syncchocystis species; ii) N-acetylmannosamine-2-epimerase from Bacteroides species, more, in particular, from Bacteroides ovatus, Bacteroides thetaiotaomicron, C. canimorsus and M. mulieris; iii) N-acetyl-D-glucosmine-2-epimerase from Glycin max, M. musculus, Homo sapiens, R. norvegicus, B. taurus, S. scrofa or C. lupus.

N-acetyl neuraminate synthase (also called sialic acid synthase in the art) (EC 2.5.1.56) activity is found in several prokaryotic organisms like, e.g., Streptococcus agalatiae, Bacillus subtilis, Legionella pneumophilla, C. jejuni, Idiomarina loihiensis, Moritella viscosa, Aliivibrio salmonicida, E. coli, Methanocaldococcus jannaschi, Clostridium sordellii, Butyrivibrio proteoclasticus, Micromonas commoda or Neisseria meningitis. Preferably, in the method and microorganism of the disclosure, the sialic acid (or N-acetyl neuraminate) synthase is selected from the group comprising: sialic acid synthase from Streptococcus agalatiae, B. subtilis, L. pneumophilla, C. jejuni, Idiomarina loihiensis, Moritella viscosa, Aliivibrio salmonicida, E. coli, Methanocaldococcus jannaschi, Clostridium sordellii, Butyrivibrio proteoclasticus, Micromonas commoda or N. meningitis.

In one preferred aspect, any one or more of the phosphatase, N-acetylmannosamine epimerase and sialic acid synthase is overexpressed in the microorganism. In an alternative preferred aspect, any one or more of the phosphatase, N-acetylmannosamine epimerase and sialic acid synthase is introduced and expressed in the microorganism.

In another aspect, the microorganism lacks the genes encoding the following enzymes i) a N-acetylglycosamine-6-phosphate deacetylase, ii) a N-acetylglucosamine kinase, and iii) a N-acetylneuraminate aldolase. In another preferred aspect, the genes encoding following enzymes i) a N-acetylglycosamine-6-phosphate deacetylase, ii) a N-acetylglucosamine kinase, and iii) a N-acetylneuraminate aldolase are reduced in activity, preferably the genes are deleted or knocked-out, in the microorganism.

In another preferred aspect, the microorganism further encodes a protein that facilitates uptake of lactose and lacks enzymes that metabolize lactose. Methods to produce microorganisms that resist lactose killing and the resulting microorganisms are described in WO2016/075243, which is herein incorporated by reference.

In a preferred aspect the microorganisms of, and used in the method of, the disclosure also express a CMP-sialic acid synthase (EC 2.7.7.43) and a sialyltransferase (EC 2.4.99.1) in order to activate the sialic acid and transfer it to a desired compound.

In a preferred aspect, the N-acetylglucosamine-6-phosphate is obtained by introducing a glucosamine-phosphate N-acetyltransferase (EC 2.3.1.4), which uses intracellular glucosamine-6-phosphate as a substrate. In most microorganisms, glucosamine-6-phosphate is naturally present in the cell, but the intracellular production can be elevated by expressing a L-glutamine:D-fructose-6-phosphate aminotransferase without inhibition, obtained either through protein engineering or by screening natural enzymes, such as present in gram positive bacteria (Deng et al. Metabolic Engineering 7 (2005), 201-214).

In the disclosure, the expression of the genes to convert N-acetylglucosamine-6-phosphate to N-acetyl-neuraminate or sialic acid is optimized in a way that enables intracellular dephosphorylation of N-acetylglucosamine-6-phosphate, prevents toxic accumulation of N-acetylglucosamine-6-phosphate and prevents excretion of N-acetylglucosamine and/or N-acetylmannosamine. The optimization is the result of the use of constitutive expression of the genes of the production pathway. In an embodiment, the disclosure prevents the excretion of at least 10%, 20%, 30%, 35%, 40%, 45%, 50%, or 60% of the formed N-acetylglucosamine and/or N-acetylmannosamine. In a further embodiment, the microorganism produces less extracellular N-acetylglucosamine and/or N-acetylmannosamine than sialylated compound. More preferably, the microorganism produces less than 50%, 40%, 30%, 20%, 10%, 5%, 2% extracellular N-acetylglucosamine and/or N-acetylmannosamine than sialylated compound. In another preferred embodiment of the disclosure the microorganism produces equal or more than 50%, 60%, 70%, 80%, 90%, 95%, 98% extracellular sialylated compound on total extracellular carbohydrate.

In a particular aspect, described is a method for synthesizing sialylated compounds, without any exogenous sialic acid addition to the culture medium.

The sialylated compound can be N-acetylneuramic acid, a sialylated oligosaccharide, a sialylated lipid, sialylated glycolipids (such as, but not limited to gangliosides, ceramides), a sialylated protein, or a sialylated aglycon.

A sialylated oligosaccharide is a charged sialic acid containing oligosaccharide, i.e., an oligosaccharide having a sialic acid residue. It has an acidic nature. Some examples are 3-SL (3-sialyllactose), 3-sialyllactosamine, 6-SL (6-sialyllactose or n-acetylneuraminate α 2,6 galactosyl β 1,4 Glucose), 6-sialyllactosamine, oligosaccharides comprising 6-sialyllactose, SGG hexasaccharide (Neu5Ac α-2,3Gal β-1,3GalNac β-1,3Gala-1,4Gal β-1,4Gal), sialylated tetrasaccharide (Neu5Ac-α-2,3Gal β-1,4GlcNAc β-14GlcNAc), pentasaccharide LSTD (Neu5Ac α-2,3Gal β-1,4GlcNAc β-1,3Gal β-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residue(s), including but not limited to: oligosaccharide moieties of the gangliosides selected from GM3 (3sialyllactose, Neu5Aca-2,3Gal β-4Glc) and oligosaccharides comprising the GM3 motif, GD3 Neu5Aca-2,8Neu5Aca-2,3Gal-1,4Glc GT3 (Neu5Aca-2,8Neu5Aca-2,8Neu5Aca-2,3Gal β-1,4Glc); GM2 GalNAc β-1,4 (Neu5Aca-2,3) Gal β-1,4Glc, GM1 Gal β-1,3GalNAc β-1,4 (Neu5Aca-2,3) Gal β-1,4Glc, GD1a Neu5Aca-2,3Gal β-1,3GalNAc β-1,4 (Neu5Aca-2,3) Gal β-1,4Glc GT1a Neu5Aca-2,8Neu5Aca-2,3Gal β-1,3GalNAc β-1,4 (Neu5Aca-2,3) Gal β-1,4Glc GD2 GalNAc β-1,4 (Neu5Aca-2,8Neu5Aca2,3) Gal β-1,4Glc GT2 GspalNAc β-1,4 (Neu5Aca-2,8Neu5Aca-2,8Neu5Aca2,3) Gal β-1,4Glc GD1b, Gal β-1,3GalNAc β-1,4 (Neu5Aca-2,8Neu5Aca2,3) Gal β-1,4Glc GT1b Neu5Aca-2,3Gal β-1,3GalNAc β-1,4 (Neu5Aca-2,8Neu5Aca2,3) Gal β-1,4Glc GQ1b Neu5Aca-2,8Neu5Aca-2,3Gal β-1,3GalNAc β-1,4 (Neu5Aca-2,8Neu5Aca2,3) Gal β-1,4Glc GT1c Gal β-1,3GalNAc β-1,4 (Neu5Aca-2,8Neu5Aca-2,8Neu5Aca2,3) Gal β-1,4Glc GQ1c, Neu5Aca-2,3Gal β-1,3GalNAc β-1,4 (Neu5Aca-2,8Neu5Aca-2,8Neu5Aca2,3) Gal β-1,4Glc GP1c Neu5Aca-2,8Neu5Aca-2,3Gal β-1,3GalNAc β-1,4 (Neu5Aca-2,8Neu5Aca-2,8Neu5Aca2,3) Gal β-1,4Glc GD1a Neu5Aca-2,3Gal β-1,3 (Neu5Aca-2,6) GalNAc β-1,4Gal β-1,4Glc Fucosyl-GM1 Fuca-1,2Gal β-1,3GalNAc β-1,4 (Neu5Aca-2,3) Gal β-1,4Glc; all of which may be extended to the production of the corresponding gangliosides by reacting the above oligosaccharide moieties with ceramide or synthetizing the above oligosaccharides on a ceramide.

The term microorganism, organism, or cell refers to a microorganism selected from the group consisting of a bacterium, a yeast, or a fungus, or refers to a plant or animal cell. The bacterium preferably belongs to the phylum of the Proteobacteria or the phylum of the Firmicutes or the phylum of the Cyanobacteria or the phylum Deinococcus-Thermus. The bacterium belonging to the phylum Proteobacteria belongs preferably to the family Enterobacteriaceae, preferably to the species E. coli. This bacterium preferably relates to any strain belonging to the species E. coli such as but not limited to E. coli B, E. coli C, E. coli W, E. coli K12, E. coli Nissle. More specifically, the term relates to cultivated E. coli strains-designated as E. coli K12 strains, which are well-adapted to the laboratory environment, and, unlike wild-type strains, have lost their ability to thrive in the intestine. Well-known examples of E. coli K12 strains are K12 Wild-type, W3110, MG1655, M182, MC1000, MC1060, MC1061, MC4100, JM101, NZN111 and AA200. Hence, the disclosure specifically relates to a mutated and/or transformed E. coli strain as indicated above wherein the E. coli strain is a K12 strain. More specifically, the disclosure relates to a mutated and/or transformed E. coli strain as indicated above wherein the K12 strain is E. coli MG1655. The latter bacterium belonging to the phylum Firmicutes belongs preferably to the Bacilli, preferably Lactobacilliales, with members such as Lactobacillus lactis, Leuconostoc mesenteroides, or Bacillales with members such as from the species Bacillus, B. subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the of the Streptomycetaceae with members Streptomyces griseus or S. fradiae. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces, Pichia, Hansenula, Kluyveromyces, Yarrowia or Starmerella. The latter fungus belongs preferably to the genus Rhizopus, Dictyostelium, Penicillium, Mucor or Aspergillus.

The culture medium for the production host can optionally comprise an exogenous precursor or this precursor can be produced by the strain itself, such as a glycan like, e.g., lactose, lactosamine, lacto-N-triose, lacto-N-tetraose, lacto-N-neotetraose; an oligosaccharide; a peptide; a lipid or an aglycon. In one particular aspect, the process of the disclosure is based on the active uptake of an exogenous precursor, such as, e.g., a mono, di or tri-saccharide, more particularly an exogenous precursor selected from lactose, N-acetyllactosamine, lacto-N-biose, galactose, β-galactoside, and α-galactoside such as but not limited to globotriose (Gal-α-1,4Gal-β-1,4Glc), while the microorganisms are growing on an inexpensive carbon substrate, such as a disaccharide such as sucrose or maltose. Moreover, these microorganisms are also able to grow on glucose, fructose or glycerol. The expression exogenous precursor is intended to denote a compound involved in the biosynthetic pathway of the product of the disclosure that is internalized by the microorganism.

In one aspect, provided is a method of producing sialylated forms of lacto-N-triose, lacto-N-tetraose, or lacto-N-neotetraose. Any one of these three molecules are synthetized by the microorganism via the activity of a galactosyltransferase (EC 2.4.1.38), preferably originating from the group comprising Homo sapiens, B. taurus, M. mulatta, Gallus gallus, Danio rerio, Helicobacter pylori and Haemophilus ducrey and/or a N-acetylglucosaminyltransferase (EC 2.4.1.90) preferably originating from the group comprising Bos Taurus, Homo Sapiens and Mus Musculus. To enhance the formation of these oligosaccharides the genes coding for UDP sugar hydrolase and galactose-1-phosphate uridylyltransferase are lacking, reducing in activity or knocked out in the microorganism.

In another aspect a method for producing a sialylated oligosaccharide is provided in which the method comprises culturing a microorganism as described above and wherein the microorganism produces internally, activated N-acetylneuraminate as donor substrate for a sialyltransferase; and wherein the method further comprises culturing the microorganism in a culture medium that comprises an exogenous precursor selected from the group consisting of lactose, N-acetyllactosamine, lacto-N-biose, galactose, β-galactoside, and α-galactoside such as but not limited to globotriose (Gal-α-1,4Gal-β-1,4Glc)galactose. The exogenous precursor is actively taken up into the microorganism and the exogenous precursor is the acceptor substrate for the sialyltransferase for producing the sialylated oligosaccharide.

In a further aspect, the method is for producing 3sialyllactose or 6sialyllactose. In this method, the microorganism is cultivated at high cell density on a carbon substrate, such as glucose or glycerol, and fed with lactose. The lactose is internalized by the lactose permease and sialylated by the recombinant sialyltransferase using the CMP-N-acetyl-neuraminate endogenously generated from N-acetylglucosamine.

The microorganism or cell of the disclosure is capable of growing on a monosaccharide, disaccharide, oligosaccharide, polysaccharide, polyol, a complex medium or a mixture thereof as the main carbon source. With the term main is meant the most important carbon source for biomass formation, carbon dioxide and/or by-products formation (such as acids and/or alcohols, such as acetate, lactate, and/or ethanol), e.g., 20, 30, 40, 50, 60, 70, 75, 80, 85, 90, 95, 98, 99% of all the required carbon is derived from the above-indicated carbon source. In one embodiment of the disclosure, the carbon source is the sole carbon source for the organism, i.e., 100% of all the required carbon is derived from the above-indicated carbon source.

In a further preferred embodiment, the microorganism or cell of the disclosure uses a split metabolism having a production pathway and a biomass pathway as described in WO2012/007481, which is herein incorporated by reference. The organism can, e.g., be genetically modified to accumulate fructose-6-phosphate by altering the genes selected from the phosphoglucoisomerase gene, phosphofructokinase gene, fructose-6-phosphate aldolase gene, fructose isomerase gene, and/or fructose:PEP phosphotransferase gene.

With the term monosaccharide is meant a sugar that is not decomposable into simpler sugars by hydrolysis, is classed as either an aldose or ketose, and contains one or more hydroxyl groups per molecule. Examples are glucose, fructose, galactose, mannose, ribose and/or arabinose.

With the term disaccharide is meant a sugar that is composed of two monosaccharides that are chemically bound. Examples are maltose, sucrose, lactose, trehalose, cellobiose and/or chitobiose.

With the term oligosaccharide is meant a sugar that is composed of three to ten monosaccharides that are chemically bound. Examples are maltotriose, fructo-oligosaccharides, galacto-oligosaccharides, mannan oligosaccharides, isomaltooligosaccharide, human milk oligosaccharides and/or glucooligosaccharides.

With the term polyol is meant an alcohol containing multiple hydroxyl groups. For example, glycerol, sorbitol, or mannitol.

With the term complex medium is meant a medium for which the exact constitution is not determined. Examples are molasses, corn steep liquor, peptone, tryptone or yeast extract.

Production of sialylated compounds can be increased by adding precursors to the medium, such as N-acetylglusosamine, N-acetylmannosamine, glutamine, glutamate, phosphoenolpyruvate and/or pyruvate.

The sialylated compounds produced in the described method may be recovered using various methods, or a combination thereof, known in the art. Depending on the produced sialylated compound, the compound is available in the extracellular fraction or retained in the cells. When the produced sialylated compound is retained in the cells, the sialylated compound will first be released from the cells by cell disruption. Again depending on the produced sialylated compound, the cells may be separated from the extracellular fraction. In the other case, cells are disrupted without first separation from the extracellular fraction, wherein cells are disrupted by techniques such as, but not limited to, heating, freeze thawing and/or shear stress through sonication, mixing and/or French press. The extracellular and/or intracellular fraction may be separated from the cells and/or cell debris by centrifugation, filtration, microfiltration, and nanofiltration. Flocculating agents may be used to aid in product separation. The sialylated compounds in the extracellular or intracellular fraction may be extracted by ion exchange, ultra- or nanofiltration or electrodialysis, chromatography such as size exclusion, ion chromatography and simulated moving bed. Another example of filtering the sialylated compounds from liquid phase is by filtration using a deep bed filter with cotton and activated carbon or carbon filter, where after the permeate is passed through a carbon polisher followed by e.g., a 0.2 micron microfiltration membrane system to remove color, microorganisms and suspended carbon particles. Thereafter the sialylated compound may be concentrated in a vacuum evaporator to obtain a concentrate. The concentrate can be precipitated and/or dried through heat drying, spray drying and/or lyophilization to obtain high purity sialylated compound. An amorphous form powder can then be obtained. This amorphous powder may further be crystallized to obtain crystalline sialylated compound.

In exemplary embodiment, sialylated compounds may be isolated from the culture medium using methods known in the art for fermentations. For example, cells may be removed from the culture medium by centrifugation, filtration, flocculation, decantation, or the like. Then, the sialylated compounds may be isolated from the extracellular fraction using methods such as ion-exchange. A further purification of the sialylated compounds may be accomplished, e.g., by nanofiltration or ultrafiltration or ion exchange to remove any remaining DNA, protein, LPS (endotoxins), or other impurity.

In another exemplary embodiment, sialyllactose may be isolated from the culture medium using, e.g., methods known in the art for fermentations. For example, cells may be removed from the culture medium by centrifugation, filtration, flocculation, decantation, or the like. Then, the sialyllactose may be isolated from the extracellular fraction using methods such as ion-exchange. A further purification of the sialyllactose may be accomplished, e.g., by nanofiltration or ultrafiltration or ion exchange to remove any remaining DNA, protein, LPS (endotoxins), or other impurity. Another purification and formulation step is accomplished by crystallization or precipitation of the product. Another formulation step is to spray dry or lyophilize sialyllactose.

The sialylated compound may contain a counter ion, such as, a monovalent ion, such as a proton, sodium ion, potassium, a divalent ion, such as calcium magnesium, iron, or, a trivalent ion such as iron, or a combination of ions.

Throughout this disclosure the term sialic acid, N-acetyl neuraminate and N-acetyl neuraminic acid are used interchangeably.

As used herein, the term intracellular or intracellularly in e.g., intracellularly converting, intracellularly production, intracellularly expressed, intracellular formed must be understood to mean within the cell of the microorganism. The term extracellular must be understood to mean outside of the cell.

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag»100 epitope, c-myc epitope, FLAG(R)-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break a-helical structures or β-sheet structures). Amino acid substitutions are typically of single residues, but may be clustered depending upon functional constraints placed upon the polypeptide and may range from 1 to 10 amino acids; insertions will usually be of the order of about 1 to 10 amino acid residues. The amino acid substitutions are preferably conservative amino acid substitutions. Conservative substitution tables are well known in the art (see, e.g., Creighton (1984) Proteins. W.H. Freeman and Company (Eds) and Table 1 below).

TABLE 1
Examples of conserved amino acid substitutions

| | Conservative | | Conservative |
|---|---|---|---|
| Residue | Substitutions | Residue | Substitutions |
| Ala | Ser | Leu | Ile; Val |
| Arg | Lys | Lys | Arg; Gln |
| Asn | Gln; His | Met | Leu; Ile |
| Asp | Glu | Phe | Met; Leu; Tyr |
| Gln | As, | Ser | Thr; Gly |
| Cys | Ser | Thr | Ser; Val |
| Glu | Asp | Trp | Tyr |
| Gly | Pro | Tyr | Trp; Phe |
| His | Asn; Gln | Val | Ile; Leu |
| Ile | Leu; Val | | |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, 17-Gen in vitro mutagenesis (USB, Cleveland, OH), QuickChange Site Directed mutagenesis (Stratagene, San Diego, CA), PCR-mediated site-directed mutagenesis or other site-directed mutagenesis protocols.

“Derivatives” include peptides, oligopeptides, polypeptides, which may, compared to the amino acid sequence of the naturally occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides, which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, e.g., a reporter molecule or other ligand, covalently or non-covalently bound to the amino acid sequence, such as a reporter molecule, which is bound to facilitate its detection, and non-naturally occurring amino acid residues relative to the amino acid sequence of a naturally-occurring protein. Furthermore, “derivatives” also include fusions of the naturally occurring form of the protein with tagging peptides such as FLAG, HIS6 or thioredoxin (for a review of tagging peptides, see Terpe, Appl. Microbiol. Biotechnol. 60, 523-533, 2003).

Orthologues and paralogues encompass evolutionary concepts used to describe the ancestral relationships of genes. Paralogues are genes within the same species that have originated through duplication of an ancestral gene, orthologues are genes from different organisms that have originated through speciation and are also derived from a common ancestral gene.

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

The term “motif” or “consensus sequence” or “signature” refers to a short, conserved region in the sequence of evolutionarily related proteins. Motifs are frequently highly conserved parts of domains, but may also include only part of the domain, or be located outside of conserved domain (if all of the amino acids of the motif fall outside of a defined domain).

Specialist databases exist for the identification of domains, e.g., SMART (Schultz et al. (1998) Proc. Natl. Acad. Sci. USA 95, 5857-5864; Letunic et al. (2002) Nucleic Acids Res 30, 242-244), InterPro (Mulder et al. (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al. Nucl. Acids. Res. 32:D134-D137, (2004)), or Pfam (Bateman et al. Nucleic Acids Research 30(1): 276-280 (2002)). A set of tools for in silico analysis of protein sequences is available on the ExPASy proteomics server (Swiss Institute of Bioinformatics (Gasteiger et al. ExPASy: the proteomics server for in-depth protein knowledge and analysis, Nucleic Acids. Res. 31:3784-3788(2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48:443-453) to find the global (i.e., spanning the complete sequences) alignment of two sequences that maximizes the number of matches and minimizes the number of gaps. The BLAST algorithm (Altschul et al. (1990) J Mol Biol 215:403-10) calculates percent sequence identity and performs a statistical analysis of the similarity between the two sequences. The software for performing BLAST analysis is publicly available through the National Centre for Biotechnology Information (NCBI). Homologues may readily be identified using, e.g., the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al. BMC Bioinformatics. 2003 Jul. 10; 4:29. MatGAT: an application that generates similarity/identity matrices using protein or DNA sequences.). Minor manual editing may be performed to optimize alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7).

Reciprocal BLAST: Typically, this involves a first BLAST involving BLASTing a query sequence (for example, using any of the sequences listed in Table A of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived. The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbor joining tree, to help visualize clustering of related genes and to identify orthologues and paralogues.

Construct(s). Additional regulatory elements may include transcriptional as well as translational enhancers. Those skilled in the art will be aware of terminator and enhancer sequences that may be suitable for use in performing the disclosure. An intron sequence may also be added to the 5 untranslated region (UTR) or in the coding sequence to increase the amount of the mature message that accumulates in the cytosol, as described in the definitions section. Other control sequences (besides promoter, enhancer, silencer, intron sequences, 3UTR and/or 5UTR regions) may be protein and/or RNA stabilizing elements. Such sequences would be known or may readily be obtained by a person skilled in the art.

The genetic constructs of the disclosure may further include an origin of replication sequence that is required for maintenance and/or replication in a specific cell type. One example is when a genetic construct is required to be maintained in a bacterial cell as an episomal genetic element (e.g., plasmid or cosmid molecule).

For the detection of the successful transfer of the nucleic acid sequences as used in the methods of the disclosure and/or selection of transgenic microorganisms comprising these nucleic acids, it is advantageous to use marker genes (or reporter genes). Therefore, the genetic construct may optionally comprise a selectable marker gene. The marker genes may be removed or excised from the transgenic cell once they are no longer needed. Techniques for marker removal are known in the art, useful techniques are described above in the definitions section.

The terms “regulatory element,” “control sequence,” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognizing and binding of RNA polymerase and other proteins, thereby directing transcription of an operably linked nucleic acid. Encompassed by the aforementioned terms are transcriptional regulatory sequences derived from a classical eukaryotic genomic gene (including the TATA box, which is required for accurate transcription initiation, with or without a CCAAT box sequence) and additional regulatory elements (i.e., upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

A “constitutive promoter” refers to a promoter that is transcriptionally active during most, but not necessarily all, phases of growth and development and under most environmental conditions, in at least one cell, tissue or organ.

For the purposes of the disclosure, “transgenic,” “transgene” or “recombinant” means with regard to, e.g., a nucleic acid sequence, an expression cassette, gene construct or a vector comprising the nucleic acid sequence or an organism transformed with the nucleic acid sequences, expression cassettes or vectors of the disclosure, all those constructions brought about by recombinant methods in which either
(a) the nucleic acid sequences encoding proteins useful in the methods of the disclosure, or
(b) genetic control sequence(s) which is operably linked with the nucleic acid sequence of the disclosure, e.g., a promoter, or
(c) a) and b) are not located in their natural genetic environment or have been modified by recombinant methods, it being possible for the modification to take the form of, e.g., a substitution, addition, deletion, inversion or insertion of one or more nucleotide residues. The natural genetic environment is understood as meaning the natural genomic or chromosomal locus in the original microorganism or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example, the naturally occurring combination of the natural promoter of the nucleic acid sequences with the corresponding nucleic acid sequence encoding a polypeptide useful in the methods of the disclosure, as defined above-becomes a transgenic expression cassette when this expression cassette is modified by non-natural, synthetic (“artificial”) methods such as, e.g., mutagenic treatment. Suitable methods are described, e.g., in U.S. Pat. No. 5,565,350 or WO 00/15815.

A transgenic microorganism for the purposes of the disclosure is thus understood as meaning, as above, that the nucleic acids used in the method of the disclosure are not present in, or originating from, the genome of the microorganism, or are present in the genome of the microorganism but not at their natural locus in the genome of the microorganism, it being possible for the nucleic acids to be expressed homologously or heterologously. However, as mentioned, transgenic also means that, while the nucleic acids of the disclosure or used in the inventive method are at their natural position in the genome of a microorganism, the sequence has been modified with regard to the natural sequence, and/or that the regulatory sequences of the natural sequences have been modified. Transgenic is preferably understood as meaning the expression of the nucleic acids of the disclosure at an unnatural locus in the genome, i.e., homologous or, preferably, heterologous expression of the nucleic acids takes place. Preferred transgenic microorganism are mentioned herein.

It shall further be noted that in the context of the disclosure, the term “isolated nucleic acid” or “isolated polypeptide” may in some instances be considered as a synonym for a “recombinant nucleic acid” or a “recombinant polypeptide,” respectively, and refers to a nucleic acid or polypeptide that is not located in its natural genetic environment and/or that has been modified by recombinant methods.

Modulation. The term “modulation” means in relation to expression or gene expression, a process in which the expression level is changed by the gene expression in comparison to the control microorganism, the expression level may be increased or decreased. The original, unmodulated expression may be of any kind of expression of a structural RNA (rRNA, tRNA) or mRNA with subsequent translation. For the purposes of this disclosure, the original unmodulated expression may also be absence of any expression. The term “modulating the activity” shall mean any change of the expression of the inventive nucleic acid sequences or encoded proteins, which leads to increased production yield and/or increased growth of the microorganisms. The expression can increase from zero (absence of, or immeasurable expression) to a certain amount, or can decrease from a certain amount to immeasurable small amounts or zero.

The term “expression” or “gene expression” means the transcription of a specific gene or specific genes or specific genetic construct. The term “expression” or “gene expression,” in particular, means the transcription of a gene or genes or genetic construct into structural RNA (rRNA, tRNA) or mRNA with or without subsequent translation of the latter into a protein. The process includes transcription of DNA and processing of the resulting mRNA product.

The term “increased expression” or “overexpression” as used herein means any form of expression that is additional to the original wild-type expression level. For the purposes of this disclosure, the original wild-type expression level might also be zero, i.e., absence of expression or immeasurable expression.

Methods for increasing expression of genes or gene products are well documented in the art and include, e.g., overexpression driven by appropriate promoters, the use of transcription enhancers or translation enhancers. Isolated nucleic acids, which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al. WO9322443), or isolated promoters may be introduced into a microorganism cell in the proper orientation and distance from a gene of the disclosure so as to control the expression of the gene.

If polypeptide expression is desired, it is generally desirable to include a polyadenylation region at the 3-end of a polynucleotide coding region. The polyadenylation region can be derived from the natural gene, from a variety of other microorganism genes, or from T-DNA.

Moreover, the disclosure relates to the following specific embodiments:

1. Method for producing sialylated compounds, the method comprising:
culturing a microorganism in a culture medium, the culture medium optionally comprising an exogenous precursor,
wherein the microorganism intracellularly converts N-acetylglucosamine-6-phosphate to N-acetylglucosamine, the N-acetylglucosamine to N-acetylmannosamine and the N-acetylmannosamine to N-acetyl-neuraminate; and
wherein the microorganism is unable to i) convert N-acetylglucosamine-6-P to glucosamine-6-P, ii) convert N-acetyl-glucosamine to N-acetyl-glucosamine-6-P, and iii) convert N-acetyl-neuraminate to N-acetyl-mannosamine.

2. The method according to embodiment 1 wherein:
i) the conversion of N-acetylglucosamine-6-phosphate to N-acetylglucosamine is obtained by the action of an intracellularly expressed phosphatase,
ii) the N-acetylglucosamine to N-acetylmannosamine conversion is performed by an intracellularly expressed N-acetylmannosamine epimerase; and
iii) intracellular expressed sialic acid synthase converts the N-acetylmannosamine to N-acetyl-neuraminate.

3. The method according to any one of embodiment 1 or 2 wherein the organism is unable to produce following enzymes i) a N-acetylglycosamine-6-phosphate deacetylase, ii) a N-acetylglucosamine kinase, and iii) a N-acetylneuraminate aldolase.

4. The method according to any one of embodiment 1 to 3, wherein all the conversions are catalyzed by enzymes encoded by constitutively expressed genes.

5. The method according to embodiment 2 wherein the phosphatase is selected from the HAD superfamily or the HAD-like phosphatase family, preferably the phosphatase is selected from the group comprising: i) enzymes expressed by the genes yqaB, inhX, yniC, ybiV, yidA, ybjI, yigL or cof from E. coli, ii) the phosphatase of B. emersonii and iii) other phosphatase families, more preferably the phosphatase is a HAD-like phosphatase polypeptide as defined herein.

6. The method according to any one of the embodiments 2, 3, 4 or 5, wherein the N-acetylmannosamine-2-epimerase is selected from the group comprising i) N-acetylmannosamine-2-epimerase from cyanobacteria, more, in particular, from A. marina, A. variabilis, Anabaena marina, Nostoc punctiforme, Acaryochloris species, Anabaena species, Nostoc species and Synechocystis species; ii) N-acetylmannosamine-2-epimerase from Bacteroides species, more, in particular, from Bacteroides ovatus, Bacteroides thetaiotaomicron, Capnocytophaga canimorsus and Mobiluncus mulieris; iii) N-acetyl-D-glucosmine-2-epimerase from Glycin max, Mus musculus, Homo sapiens, Rattus norvegicus, Bos Taurus, Sus scrofa or Canis lupus.

7. The method according to any one of the embodiments 2, 3, 4, 5 or 6, wherein the sialic acid synthase is selected from the group comprising: sialic acid synthase from Streptococcus agalatiae, B. subtilis, L. pneumophilla, C. jejuni, Idiomarina loihiensis, Moritella viscosa, Aliivibrio salmonicida, E. coli, Methanocaldococcus jannaschi, Clostridium sordellii, Butyrivibrio proteoclasticus, Micromonas commoda or N. meningitis.

8. The method according to any one of the preceding embodiments, wherein the sialylated compound is selected from the group consisting of N-acetylneuramic acid, sialylated oligosaccharide, sialylated lipids, sialylated protein, sialylated aglycon.

9. The method according to the previous embodiment, wherein the sialylated compound is a sialylated oligosaccharide.

10. The method according to embodiment 9, wherein the sialylated oligosaccharide is sialyllactose, preferably any one of 3-SL or 6-SL.

11. The method according to embodiment 9, wherein the sialylated oligosaccharide is disialyl lacto-N-tetraose.

12. The method according to embodiment 8, wherein the sialylated compound is N-acetylneuraminic acid.

13. The method according to any one of embodiment 1 to 10 wherein the sialylated compound is a sialylated lacto-N-triose, lacto-N-tetraose or a lacto-N-neotetraose, and wherein the microorganism further comprises the activity of a galactosyltransferase (EC 2.4.1.38), preferably the galactosyltransferase originates from the group comprising Homo sapiens, Bos taurus, Mus mulatta, Gallus gallus, Danio rerio, H. pylori and Haemophilus ducrey; and/or the microorganism comprises the activity of a N-acetylglucosaminyltransferase (EC 2.4.1.90), preferably the N-acetylglucosaminyltransferase originates from the group comprising Bos taurus, Homo sapiens and Mus musculus.

14. The method according to embodiment 13 wherein the microorganism is unable to express the genes coding for UDP sugar hydrolase and galactose-1-phosphate uridylyltransferase.

15. The method according to any one of embodiments 1 to 14, wherein the microorganism produces less than 50%, 40%, 30%, 20%, 10%, 5%, 2% extracellular N-acetylglucosamine and/or N-acetylmannosamine than sialylated compound and/or the microorganism produces equal or more than 50%, 60%, 70%, 80%, 90%, 95%, 98% sialylated compound on total carbohydrate.

16. A method for producing a sialylated oligosaccharide, comprising:
a) culturing a microorganism according to the method of any one of embodiments 1 to 7, 14 and 15, and wherein the microorganism produces internally, activated N-acetylneuraminate as donor substrate for a sialyltransferase; and
b) culturing the microorganism in a culture medium comprising an exogenous precursor selected from the group consisting of lactose, N-acetyllactosamine, lacto-N-biose, galactose, β-galactoside, and α-galactoside such as but not limited to globotriose (Gal-α-1,4Gal-β-1,4Glc)galactose, wherein active uptake into the microorganism of the exogenous precursor occurs and wherein the exogenous precursor is the acceptor substrate for the sialytransferase for producing the sialylated oligosaccharide.

17. The method according to embodiment 2, wherein any one or more of the phosphatase, N-acetylmannosamine epimerase and sialic acid synthase is overexpressed in the microorganism.

18. The method according to embodiment 2, wherein any one or more of the phosphatase, N-acetylmannosamine epimerase and sialic acid synthase is introduced and expressed in the microorganism.

19. The method according to embodiment 3, wherein the microorganism lacks the genes encoding following enzymes i) a N-acetylglycosamine-6-phosphate deacetylase, ii) a N-acetylglucosamine kinase, and iii) a N-acetylneuraminate aldolase.

20. The method according to embodiment 3, wherein in the microorganism the genes encoding following enzymes i) a N-acetylglycosamine-6-phosphate deacetylase, ii) a N-acetylglucosamine kinase, and iii) a N-acetylneuraminate aldolase are reduced in activity, preferably the genes are deleted or knocked-out.

21. The method according to any one of the embodiments 1 to 20, wherein the microorganism further encodes a protein that facilitates uptake of lactose and lacks enzymes that metabolize lactose.

22. The method according to any one of embodiments 1 to 21, wherein the microorganism is a bacterium, preferably an E. coli strain, more preferably an E. coli strain, which is a K12 strain, even more preferably the E. coli K12 strain is E. coli MG1655.

23. The method according to any one of embodiments 1 to 21, wherein the microorganism is a yeast.

24. The method according to any one of embodiments 1 to 23, wherein the exogenous precursor is selected from the group comprising lactose, galactose, β-galactoside, and α-galactoside, such as globotriose (Gal-α-1,4Gal-β-1,4Glc).

25. A microorganism for the production of sialylated compounds, the microorganism
intracellularly converts N-acetylglucosamine-6-phosphate to N-acetylglucosamine, the N-acetylglucosamine to N-acetylmannosamine and the N-acetylmannosamine to N-acetyl-neuraminate; and
is unable to i) convert N-acetylglucosamine-6-P to glucosamine-6-P, ii) convert N-acetyl-glucosamine to N-acetyl-glucosamine-6-P, and iii) convert N-acetyl-neuraminate to N-acetyl-mannosamine.

26. A microorganism for the production of a sialylated compound, the microorganism being defined in any one of embodiments 2 to 24.

27. A cell culture medium comprising lactose as precursor and the microorganism of any one of embodiments 25 or 26.

28. The method according to one of embodiments 1 to 24, for the production of 3sialyllactose or 6sialyllactose, wherein the microorganism is cultivated at high cell density on a carbon substrate, such as glucose or glycerol, and fed with lactose, which is internalized by the lactose permease and sialylated by the recombinant sialyltransferase using the CMP-N-acetyl-neuraminate endogenously generated from N-acetylglucosamine.

29. The method according to any one of embodiments 1 to 24, wherein the sialylated compound is isolated from the culture medium by way of a unit operation selected from the group centrifugation, filtration, microfiltration, ultrafiltration, nanofiltration, ion exchange, electrodialysis, chromatography, simulated moving bed, evaporation, precipitation, crystallization, lyophilization and/or spray drying.

30. A sialylated compound produced according to the method described in any one of embodiments 1 to 24, wherein the sialylated compound is purified by centrifugation and/or filtration, ion-exchange, concentration through evaporation or nanofiltration, formulation through crystallization or spray drying or lyophilization.

31. A sialylated compound produced according to the method described in any one of embodiments 1 to 24, wherein the sialylated compound is added to food formulation, feed formulation, pharmaceutical formulation, cosmetic formulation, or agrochemical formulation.

32. The method according to any one of embodiments 1 to 24, wherein the culture medium comprises any one or more of the following: a monosaccharide, disaccharide, oligosaccharide, polysaccharide, polyol, a complex medium as the main carbon source.

33. The method according to embodiment 32, wherein the main carbon source provides at least 20%, 30%, 40%, 50%, 60%, 70%, 75%, 80%, 85%, 90%, 95%, 98%, 99% or 100% of all required carbon for the growth of the microorganism.

34. The method according to embodiment 32, wherein the monosaccharide is selected from the group comprising glucose, fructose, galactose, mannose, ribose or arabinose.

35. The method according to embodiment 32, wherein the disaccharide is selected from the group comprising maltose, sucrose, lactose, trehalose, cellobiose or chitobiose.

36. The method according to embodiment 32, wherein the oligosaccharide is selected from the group comprising maltotriose, fructo-oligosaccharides, galacto-oligosaccharides, mannan oligosaccharides, isomaltooligosaccharide or glucooligosaccharides.

37. The method according to embodiment 32, wherein the polyol is selected from the group comprising glycerol.

38. The method according to embodiment 32, wherein the complex medium is selected from the group comprising molasses, corn steep liquor, peptone, tryptone or yeast extract.

In a preferred aspect, the disclosure relates to the following preferred specific embodiments:

1. A method for producing a sialylated compound in a microorganism, the method comprising:
culturing a microorganism in a culture medium, the culture medium optionally comprising an exogenous precursor,
wherein the microorganism comprises at least one nucleic acid encoding a phosphatase, at least one nucleic acid encoding an N-acetylmannosamine epimerase; and at least one nucleic acid encoding a sialic acid synthase, and
wherein the microorganism is unable to i) convert N-acetylglucosamine-6-P to glucosamine-6-P, ii) convert N-acetyl-glucosamine to N-acetyl-glucosamine-6-P, and iii) convert N-acetyl-neuraminate to N-acetyl-mannosamine; and
modulating expression in the microorganism of a nucleic acid encoding a HAD-like phosphatase polypeptide, wherein the HAD-like phosphatase polypeptide comprises:
at least one of the following motifs:

Motif 1:
(SEQ ID NO: 73)
hDxDx[TV],
or
Motif 2:
(SEQ ID NOs: 74, 75, 76, 77)
[GSTDE][DSEN]x(1-2)[hP]x(1-2)[DGTS]

wherein h means a hydrophobic amino acid (A, I, L, M, F, V, P, G) and x can be any distinct amino acid;
or a homologue or derivative of any one of SEQ ID NOs: 43, 44, 45, 47, 48, 50, 51, 52, 54, 55 or 57 having at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to the polypeptide.

2. The method according to preferred embodiment 1, wherein the HAD-like polypeptide comprises any one of SEQ ID NOs: 43,44, 45, 47, 48, 50, 51, 52, 54, 55, 57.

3. Method according to preferred embodiment 1, wherein the modulated expression is affected by introducing and expressing in a microorganism a nucleic acid encoding a HAD-like polypeptide.

4. Method according to preferred embodiment 1, wherein the modulated expression is effected by the action of a constitutive promoter.

5. The method according to any one of the preceding preferred embodiments, wherein the sialylated compound is selected from the group consisting of N-acetylneuramic acid, sialylated oligosaccharide, sialylated lipids, sialylated protein, sialylated aglycon.

6. The method according to the previous preferred embodiment, wherein the sialylated compound is a sialylated oligosaccharide.

7. The method according to preferred embodiment 8, wherein the sialylated oligosaccharide is sialyllactose.

8. The method according to preferred embodiment 8, wherein the sialylated oligosaccharide is disialyl lacto-N-tetraose.

9. The method according to preferred embodiment 7, wherein the sialylated compound is N-acetylneuraminic acid.

10. The method according to any one of preferred embodiment 1 to 9 wherein the sialylated compound is a sialylated lacto-N-triose, lacto-N-tetraose or a lacto-N-neotetraose, and wherein the microorganism further comprises the activity of a galactosyltransferase (EC 2.4.1.38), preferably the galactosyltransferase originates from the group comprising Homo sapiens, Bos taurus, Mus mulatta, Gallus gallus, Danio rerio, H. pylori and Haemophilus ducrey; and/or the microorganism comprises the activity of a N-acetylglucosaminyltransferase (EC 2.4.1.90), preferably the N-acetylglucosaminyltransferase originates from the group comprising Bos taurus, Homo sapiens and Mus musculus.

11. The method according to preferred embodiment 12 wherein the microorganism is unable to express the genes coding for UDP sugar hydrolase and galactose-1-phosphate uridylyltransferase.

12. The method according to any one of preferred embodiments 1 to 13, wherein the microorganism produces less than 50%, 40%, 30%, 20%, 10%, 5%, 2% extracellular N-acetylglucosamine and/or N-acetylmannosamine than sialylated compound and/or the microorganism produces equal or more than 50%, 60%, 70%, 80%, 90%, 95%, 98% sialylated compound on total carbohydrate.

13. A method for producing a sialylated oligosaccharide, comprising:
a) culturing a microorganism according to the method of any one of preferred embodiments 1 to 12, and wherein the microorganism produces internally, activated N-acetylneuraminate as donor substrate for a sialyltransferase; and
b) culturing the microorganism in a culture medium comprising an exogenous precursor selected from the group consisting of lactose, N-acetyllactosamine, lacto-N-biose, galactose, β-galactoside, and α-galactoside such as but not limited to globotriose (Gal-α-1,4Gal-β-1,4Glc)galactose, wherein active uptake into the microorganism of the exogenous precursor occurs and wherein the exogenous precursor is the acceptor substrate for the sialyltransferase for producing the sialylated oligosaccharide.

14. The method according to preferred embodiment 1, wherein any one or more of the N-acetylmannosamine epimerase and sialic acid synthase is overexpressed in the microorganism.

15. The method according to preferred embodiment 1, wherein any one or more of the N-acetylmannosamine epimerase and sialic acid synthase is introduced and expressed in the microorganism.

16. The method according to preferred embodiment 1, wherein the microorganism lacks the genes encoding following enzymes i) a N-acetylglycosamine-6-phosphate deacetylase, ii) a N-acetylglucosamine kinase, and iii) a N-acetylneuraminate aldolase.

17. The method according to preferred embodiment 1, wherein in the microorganism the genes encoding following enzymes i) a N-acetylglycosamine-6-phosphate deacetylase, ii) a N-acetylglucosamine kinase, and iii) a N-acetylneuraminate aldolase are reduced in activity, preferably the genes are deleted or knocked-out.

18. The method according to any one of the preferred embodiments 1 to 17, wherein the microorganism further encodes a protein that facilitates uptake of lactose and lacks enzymes that metabolize lactose.

19. The method according to any one of preferred embodiments 1 to 18, wherein the microorganism is a bacterium, preferably an E. coli strain, more preferably an E. coli strain, which is a K12 strain, even more preferably the E. coli K12 strain is E. coli MG1655.

20. The method according to any one of preferred embodiments 1 to 18, wherein the microorganism is a yeast.

21. The method according to any one of preferred embodiments 1 to 20, wherein the exogenous precursor is selected from the group comprising lactose, galactose, β-galactoside, and α-galactoside, such as globotriose (Gal-α-1,4Gal-β-1,4Glc).

22. Microorganism, obtainable by a method according to any one of embodiments 1 to 21, wherein the microorganism comprises a recombinant nucleic acid encoding a HAD-like polypeptide.

23. A microorganism for the production of sialylated compounds wherein the microorganism comprises at least one nucleic acid encoding a phosphatase, at least one nucleic acid encoding an N-acetylmannosamine epimerase; and at least one nucleic acid encoding a sialic acid synthase, and wherein the microorganism is unable to i) convert N-acetylglucosamine-6-P to glucosamine-6-P, ii) convert N-acetyl-glucosamine to N-acetyl-glucosamine-6-P, and iii) convert N-acetyl-neuraminate to N-acetyl-mannosamine; characterized in that the microorganism comprises a modulated expression of a nucleic acid encoding a HAD-like phosphatase polypeptide as defined in preferred embodiment 1.

24. Construct comprising:
(i) nucleic acid encoding a HAD-like polypeptide as defined in preferred embodiment 1 or 2;
(ii) one or more control sequences capable of driving expression of the nucleic acid sequence of (i); and optionally
(iii) a transcription termination sequence.

25. Construct according to preferred embodiment 24, wherein one of the control sequences is a constitutive promoter.

26. Use of a construct according to preferred embodiment 24 or 25 in a method for producing sialylated compounds.

27. A sialylated compound produced according to the method described in any one of preferred embodiments 1 to 21, wherein the sialylated compound is added to food formulation, feed formulation, pharmaceutical formulation, cosmetic formulation, or agrochemical formulation.

28. A microorganism for the production of a sialylated compound, the microorganism being defined in any one of embodiments 2 to 21.

29. A cell culture medium comprising lactose as precursor and the microorganism of any one of embodiments 22, 23 or 28.

30. The method according to one of embodiments 1 to 21, for the production of 3sialyllactose or 6sialyllactose, wherein the microorganism is cultivated at high cell density on a carbon substrate, such as glucose or glycerol or sucrose, and fed with lactose, which is internalized by the lactose permease and sialylated by the recombinant sialyltransferase using the CMP-N-acetyl-neuraminate endogenously generated from N-acetylglucosamine.

31. The method according to any one of embodiments 1 to 21, wherein the sialylated compound is isolated from the culture medium by way of a unit operation selected from the group centrifugation, filtration, microfiltration, ultrafiltration, nanofiltration, ion exchange, electrodialysis, chromatography, simulated moving bed, evaporation, precipitation, crystallization, lyophilization and/or spray drying.

32. A sialylated compound produced according to the method described in any one of embodiments 1 to 21, wherein the sialylated compound is purified by centrifugation and/or filtration, ion-exchange, concentration through evaporation or nanofiltration, formulation through crystallization or spray drying or lyophilization.

33. A sialylated compound produced according to the method described in any one of embodiments 1 to 21, wherein the sialylated compound is added to food formulation, feed formulation, pharmaceutical formulation, cosmetic formulation, or agrochemical formulation.

34. The method according to any one of embodiments 1 to 21, wherein the culture medium comprises any one or more of the following: a monosaccharide, disaccharide, oligosaccharide, polysaccharide, polyol, a complex medium as the main carbon source.

35. The method according to embodiment 34, wherein the main carbon source provides at least 20%, 30%, 40%, 50%, 60%, 70%, 75%, 80%, 85%, 90%, 95%, 98%, 99% or 100% of all required carbon for the growth of the microorganism.

36. The method according to embodiment 34, wherein the monosaccharide is selected from the group comprising glucose, fructose, galactose, mannose, ribose or arabinose.

37. The method according to embodiment 34, wherein the disaccharide is selected from the group comprising maltose, sucrose, lactose, trehalose, cellobiose or chitobiose.

38. The method according to embodiment 34, wherein the oligosaccharide is selected from the group comprising maltotriose, fructo-oligosaccharides, galacto-oligosaccharides, mannan oligosaccharides, isomaltooligosaccharide or glucooligosaccharides.

39. The method according to embodiment 34, wherein the polyol is selected from the group comprising glycerol.

40. The method according to embodiment 34, wherein the complex medium is selected from the group comprising molasses, corn steep liquor, peptone, tryptone or yeast extract.

The following drawings and examples will serve as further illustration and clarification of the disclosure and are not intended to be limiting.

### BRIEF DESCRIPTION OF THE DRAWINGS

FIG. 1 shows an exemplary pathway as used in Example 2 for the production of sialic acid of the disclosure. FIG. 1A shows the pathway without all KO and overexpression signs. FIG. 1B shows the pathway as used in Example 2 with the knock-out indicated with a cross and overexpression with an upgoing arrow next to the indicated enzyme.

FIG. 2 shows the production results of the E. coli strain capable of producing sialic acid as described in Example 2.

FIG. 3 shows examples of different sialylated compounds that can be produced in the method of the disclosure.

FIG. 4 shows the optical density and sialic acid production of strains supplemented with the indicated phosphatases.

FIG. 5 shows the growth rates of strains supplemented with the indicated phosphatases.

FIG. 6 illustrates portions of a multiple-sequence alignment of the phosphatases tested in the Examples. From top to bottom, the alignment includes SEQ ID NOs: 58, 53, 42, 67, 55, 60, 49, 51, 43, 62, 47, 44, 61, 72, 63, 45, 65, 64, 46, 68, 54, 52, 69, 48, 56, 66, 57, 59, 50, and 71; the same thirty sequences appear in both Motif 1 and Motif 2 panels.

### DETAILED DESCRIPTION

### Example 1: Materials and Methods

### Method and Materials E. coli

Media: Three different media were used, namely a rich Luria Broth (LB), a minimal medium for shake flask (MMsf) and a minimal medium for fermentation (MMf). Both minimal media use a trace element mix.

Trace element mix consisted of 3.6 g/L FeCl2·4H20, 5 g/L CaCl2·2H20, 1.3 g/L MnCl2·2H20, 0.38 g/L CuCl2·2H20, 0.5 g/L CoCl2·6H20, 0.94 g/L ZnCl2, 0.0311 g/L H3B04, 0.4 g/L Na2EDTA·2H20 and 1.01 g/L thiamine·HCl. The molybdate solution contained 0.967 g/L Na2Mo04·2H20. The selenium solution contained 42 g/L Se02.

The Luria Broth (LB) medium consisted of 1% tryptone peptone (Difco, Erembodegem, BE), 0.5% yeast extract (Difco) and 0.5% sodium chloride (VWR, Leuven, BE).

Luria Broth agar (LBA) plates consisted of the LB media, with 12 g/L agar (Difco, Erembodegem, BE) added.

Minimal medium for shake flask experiments (MMsf) contained 2.00 g/L NH4CI, 5.00 g/L (NH4)2S04, 2.993 g/L KH2P04, 7.315 g/L K2HP04, 8.372 g/L MOPS, 0.5 g/L NaCI, 0.5 g/L MgS04·7H20. A carbon source chosen from, but not limited to glucose, fructose, maltose, glycerol and maltotriose, was used. The concentration was default 15 g/L, but this was subject to change depending on the experiment. 1 mL/L trace element mix, 100 μL/L molybdate solution, and 1 mL/L selenium solution. The medium was set to a pH of 7 with IM KOH. Depending on the experiment lactose could be added as a precursor.

The minimal medium for fermentations contained 6.75 g/L NH4CI, 1.25 g/L (NH4)2S04, 1.15 g/L KH₂P04 (low phosphate medium) or 2.93 g/L KH₂P04 and 7.31 g/L KH2P04 (high phosphate medium), 0.5 g/L NaCI, 0.5 g/L MgS04·7H20, a carbon source including but not limited to glucose, sucrose, fructose, maltose, glycerol and maltotriose, 1 mL/L trace element mix, 100 μL/L molybdate solution, and 1 mL/L selenium solution with the same composition as described above.

Complex medium, e.g., LB, was sterilized by autoclaving (121° C., 21) and minimal medium (MMsf and MMf) by filtration (0.22 μm Sartorius). If necessary the medium was made selective by adding an antibiotic (e.g., ampicillin (100 mg/L), chloramphenicol (20 mg/L), carbenicillin (100 mg/L), spectinomycin (40 mg/L) and/or kanamycin (50 mg/L)).

Strains: E. coli MG1655 [lambda⁻, F⁻, rph-1] was obtained from Coli Genetic Stock Center (US), CGSC Strain #: 7740 in March 2007. Mutant strains were constructed using the homologous recombination, as described by Datsenko and Wanner (PNAS 97 (2000), 6640-6645).

Plasmids: pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, BE in 2007).

Plasmid pCX-CjneuB was constructed using Gibson assembly. The gene CjneuB1 was expressed using the expression vector as described by Aerts et al. (Eng. Life Sci. 2011, 11, No. 1, 10-19).

Plasmid pCX-CjneuB-NmneuA-Pdbst was constructed using Gibson assembly. The genes CjneuB1, NmneuA and Pabst were expressed using the expression vector as described by Aerts et al. (2011).

Plasmids for phosphatase expression were constructed using Golden Gate assembly. The phosphatases (EcAphA, EcCof, EcHisB, EcOtsB, EcSurE, EcYaed, EcYcjU, EcYedP, EcYfbT, EcYidA, EcYigB, EcYihX, EcYniC, EcYqaB, EcYrbL and PsMupP) were expressed using promoters apFAB87 and apFAB346 and UTRs gene 10_SD2-junction_HisHA and UTR1 AATTCGCCGGAGGGATATTAAAAtgaatggaaaattgAAACATCTTAATCATGCTAAGGAGG TTTTCTAATG (SEQ ID NO:41). All promoters and UTRs except UTR1 are described by Mutalik et. al (Nat. Methods 2013, No. 10, 354-360). Also phosphatases EcAppA, EcGph, EcSerB, EcNagD, EcYbhA, EcYbiV, EcYbjL, EcYfbR, EcYicH, EcYjgL, EcYjjG, EcYrfG, EcYbiU, ScDOG1 and BsAraL are expressed using the same promoters and UTRs.

Plasmid pBR322-NmneuB was constructed using a pBR322 vector via Golden Gate assembly. The promoter and UTR used for the expression of NmNeuB are promoter apFAB299 and UTR galE_SD2-junction_BCD12. Plasmid pSC101-NmneuA-Pdbst was constructed using a pSC101 vector via Golden Gate assembly. The promoters and UTRs used for the expression of NmneuA are promoter apFAB37 and UTR galE_SD2-junction_BCD18. The promoters and UTRs used for the expression of Pdbst are promoter apFAB339 and UTR galE_SD2-junction_BCD12. All promoters and UTRs are described by Mutalik et. al (Nat. Methods 2013, No. 10, 354-360).

Plasmids were maintained in the host E. coli DH5α (F, phi80dlacZdeltaM15, delta(lacZYA-argF) U169, deoR, recA1, endA1, hsdR17 (rk⁻, mk⁺), phoA, supE44, lambda⁻, thi-1, gyrA96, relA1). Bought from Invitrogen.

Gene disruptions: Gene disruptions as well as gene introductions were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain.

In Table A the necessary primers for the construction of the gene disruption cassette are listed.

TABLE A
Lists of primers to construct disruption cassette for the target gene.

| Gene | | |
|---|---|---|
| target | Fw primer | Rv primer |
| lacZYA | GCTGAACTTGTAGGCCTGATA | GCGCAACGCAATTAATGTGA |
| | AGCGCAGCGTATCAGGCAATT | GTTAGCTCACTCATTAGGCAC |
| | TTTATAATCTTCATTTAAATG | CCCAGGCTTCGCCTACCTGTG |
| | GCGCGC (SEQ ID NO: 1) | ACGGAAG (SEQ ID NO: 2) |
| nagABCD | CGCTTAAAGATGCCTAATCCG | GGCGTTTGTCATCAGAGCCA |
| E | CCAACGGCTTACATTTTACTT | ACCACGTCCGCAGACGTGGT |
| | ATTGAGGTGAATAGTGTAGGC | TGCTATCATATGAATATCCTC |
| | TGGAGCTGCTTC (SEQ ID NO: 3) | CTTAG (SEQ ID NO: 4) |
| nanATEK | TAATGCGCCGCCAGTAAATCA | CCAACAACAAGCACTGGATA |
| | ACATGAAATGCCGCTGGCTCC | AAGCGAGTCTGCGTCGCCTG |
| | GTGTAGGCTGGAGCTGCTTC | GTTCAGTTCACATATGAATAT |
| | (SEQ ID NO: 5) | CCTCCTTAG (SEQ ID NO: 6) |
| manXYZ | AAAATACATCTGGCACGTTGA | CCTCCAGATAAAAAAACGGG |
| | GGTGTTAACGATAATAAAGG | GCCAAAAGGCCCCGGTAGTG |
| | AGGTAGCAAGTGTAGGCTGG | TACAACAGTCCATATGAATAT |
| | AGCTGCTTC (SEQ ID NO: 7) | CCTCCTTAG (SEQ ID NO: 8) |

For the genomic integration of the necessary genes into the production hosts genome based on the same technique used for the gene disruption, discussed before, with specific alterations to the disruption cassette. Between a homology site and the FRT site of the disruption cassette, the to be integrated constructed is located. This allows for elegant integration of the constructed in the region dictated by the homology sites.

Using this workflow, a direct gene disruption and genomic integration is possible. Primers that were used for target integration are at specific sites are listed in Table B.

TABLE B
Primers used for genomic integration

| Integration | | |
|---|---|---|
| location | Fw primer | Rv primer |
| nagABCDE | GTTTGGCGTTTGTCATCAG | TTGTCATTGTTGGATGCGA |
| | AGCCAACCACGTCCGCAG | CGCTCAAGCGTCGCATCAG |
| | ACGTGGTTGCTATGTGTAG | GCATAAAGCAGACTTAAGC |
| | GCTGGAGCTGCTTC (SEQ | GACTTCATTCACC (SEQ ID |
| | ID NO: 9) | NO: 10) |
| nanATEK | CATGGCGGTAATGCGCCG | CCAACAACAAGCACTGGAT |
| | CCAGTAAATCAACATGAA | AAAGCGAGTCTGCGTCGCC |
| | ATGCCGCTGGCTCCGTGTA | TGGTTCAGTTCACTTAAGC |
| | GGCTGGAGCTGCTTC (SEQ | GACTTCATTCACC (SEQ ID |
| | ID NO: 11) | NO: 12) |
| manXYZ | AAAATACATCTGGCACGTT | CCTCCAGATAAAAAAACGG |
| | GAGGTGTTAACGATAATA | GGCCAAAAGGCCCCGGTA |
| | AAGGAGGTAGCAAGTGTA | GTGTACAACAGTCCTTAAG |
| | GGCTGGAGCTGCTTC (SEQ | CGACTTCATTCACC (SEQ ID |
| | ID NO: 13) | NO: 14) |
| lacZYA | GCGCAACGCAATTAATGT | GCTGAACTTGTAGGCCTGA |
| | GAGTTAGCTCACTCATTAG | TAAGCGCAGCGTATCAGGC |
| | GCACCCCAGGCTTGTGTAG | AATTTTTATAATCTTAAGC |
| | GCTGGAGCTGCTTC (SEQ | GACTTCATTCACC (SEQ ID |
| | ID NO: 15) | NO: 16) |
| atpI-gidB | CAAAAAGCGGTCAAATTA | ATAACGTGGCTTTTTTTGGT |
| | TACGGTGCGCCCCCGTGAT | AAGCAGAAAATAAGTCATT |
| | TTCAAACAATAAGGTGTA | AGTGAAAATATCTTAAGCG |
| | GGCTGGAGCTGCTTC | ACTTCATTCACC |
| | (SEQ ID NO: 17) | (SEQ ID |
| | | NO: 18) |

Clones carrying the temperature sensitive pKD46 helper plasmid were grown in 10 mL LB media with ampicillin (100 mg/L) and L-arabinose (10 mM) at 30° C. to an OD₆₀₀ₙₘ of 0.6. The cells were made electro competent by sequential washing, once with 50 mL, and once with 1 mL ice-cold deionized water. Next, the cells were resuspended in 50 μL of ice-cold water. Finally, 10-100 ng of disruption/integration cassette was added to 50 μL of the washed cell solution for electroporation. Electroporation was performed using a Gene Pulser (trademark of BioRad) (600 Ohm 25 μFD, and 250 V).

After electroporation, cells were resuscitated in 1 mL LB media for 1 h at 37° C., and finally plated out onto LB-agar containing 25 mg/L of chloramphenicol or 50 mg/L of kanamycin to select antibiotic resistant transformants. The selected mutants were verified by PCR with primers upstream and downstream of the modified region and were subsequently grown on LB-agar at 42° C. for the loss of the pKD46 helper plasmid. The mutants were finally tested for ampicillin sensitivity.

The selected mutants (chloramphenicol or kanamycin resistant) were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistances and thus also of the FLP helper plasmid. The gene disruptions and/or gene integration are checked with control primers and sequenced. These primers are listed in Table C.

TABLE C
Primers to validate either gene disruption
and/or genomic integration
for specific gene targets.

| Gene | | |
|---|---|---|
| targets | Fw primer | Rv primer |
| lacZYA | CAGGTTTCCC | TGTGCGTCGT |
| | GACTGGAAAG | TGGGCTGATG |
| | (SEQ ID NO: 19) | (SEQ ID NO: 20) |
| nagABCDE | CGCTTGTCAT | GCTGACAAAGT |
| | TGTTGGATGC | GCGATTTGTTC |
| | (SEQ ID NO: 21) | (SEQ ID NO: 22) |
| nanATEK | GTCGCCCTGT | CTTTCGGTCA |
| | AATTCGTAAC | GACCACCAAC |
| | (SEQ ID NO: 23) | (SEQ ID NO: 24) |
| manXYZ | ACGCCTCTGA | AGCCAGTGCG |
| | TTTGGCAAAG | CTTAATAACC |
| | (SEQ ID NO: 25) | (SEQ ID NO: 26) |
| atpI- | GCTGAACAGCA | TGAACGATAT |
| gidB | ATCCACTTG | GGTGAGCTGG |
| | (SEQ ID NO: 27) | (SEQ ID NO: 28) |

### Heterologous and Homologous Expression

Genes that needed to be expressed, be it from a plasmid or from the genome were synthetically synthetized with one of the following companies: DNA2.0, Gen9 or IDT.

E. coli native genes, as e.g., phosphatases, were picked from the E. coli K-12 MG1655 genome. The origin of other genes are indicated in the relevant table.

Expression could be further facilitated by optimizing the codon usage to the codon usage of the expression host. Gene were optimized using the tools of the supplier.

Cultivation conditions: A preculture of 96well microtiter plate experiments was started from single colony on a LB plate, in 175 μL and was incubated for 8 h at 37° C. on an orbital shaker at 800 rpm. This culture was used as inoculum for a 96well microtiter plate, with 175 μL MMsf medium by diluting 300×. These cultures in turn, were used as a preculture for the final experiment in a 96well plate, again by diluting 300×. The 96well plate can either be microtiter plate, with a culture volume of 175 μL or a 24well deepwell plate with a culture volume of 3 mL.

A preculture for shake flask experiments was started from a single colony on a LB-plate, in 5 mL LB medium and was incubated for 8 h at 37° C. on an orbital shaker at 200 rpm. From this culture, 1 mL was transferred to 100 mL minimal medium (MMsf) in a 500 mL shake flask and incubated at 37° C. on an orbital shaker at 200 rpm. This setup is used for shake flask experiments.

A shake flask experiment grown for 16h could also be used as an inoculum for a bioreactor. 4% of this cell solution was to inoculate a 2 L Biostat Dcu-B with a 4 L working volume, controlled by MFCS control software (Sartorius Stedim Biotech, Melsungen, Germany). Culturing condition were set to 37° C., 800 rpm stirring, and a gas flow rate of 1.5 L/min. The pH was controlled at 7 using 0.5 M H2S04 and 25% NH4OH. The exhaust gas was cooled. 10% solution of silicone antifoaming agent was added when foaming raised during the fermentation (approximately 10 6 L). The use of an inducer is not required as all genes are constitutively expressed.

### Material and Methods Saccharomyces cerevisae

Media: Strains are grown on Synthetic Defined yeast medium with Complete Supplement Mixture (SD CSM) or CSM drop-out (SD CSM-Ura) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura (MP Biomedicals).

Strains: The S. cerevisiae BY4742 (Bachmann et al. (Yeast (1998) 14:115-32)) used was from the Euroscarf Culture Collection. All mutant strains were created by homologous recombination or plasmid transformation using the method of Gietz (Yeast 11:355-360, 1995). Kluyveromyces marxianus lactis is available from the LMG Culture Collection (Ghent, BE).

Plasmids: Yeast expression plasmid p2a_2μ_sia_GFA1 (Chan 2013 (Plasmid 70 (2013) 2-17)) was used for expressing foreign genes in S. cerevisiae. This plasmid contains an ampicillin resistance gene and a bacterial origin of replication to allow for selection and maintenance in E. coli. The plasmid further contains the 2μ yeast ori and the Ura3 selection marker for selection and maintenance in yeast. Finally, the plasmid can contain a β-galactosidase expression cassette. This plasmid also contains a N-acetylglucosamine-2-epimerase (e.g., from Bacteroides ovatus (BoAGE)) and a sialic acid synthase (for example, from C. jejuni (CjneuB)). Finally, it also contains the fructose-6-P-aminotransferase from S. cerevisiae, ScGFA1.

Yeast expression plasmid p2a_2μ_sia_glmS is based on p2a_2μ_sia but modified in a way that also glmS*54 (fructose-6-P-aminotransferase from E. coli) is expressed.

Yeast expression plasmids p2a_2μ_sia_glmS_phospha is based on p2a_2μ_sia_glmS but modified in a way that also EcAphA (SEQ ID NO:42), EcCof (SEQ ID NO: 43), EcHisB (SEQ ID NO:44), EcOtsB (SEQ ID NO:45), EcSurE (SEQ ID NO:46), EcYaed (SEQ ID NO:47), EcYcjU (SEQ ID NO:48), EcYedP (SEQ ID NO:49), EcYfbT (SEQ ID NO:50), EcYidA (SEQ ID NO:51), EcYigB (SEQ ID NO:52), EcYihX (SEQ ID NO:53), EcYniC (SEQ ID NO: 54), EcYqaB (SEQ ID NO:55), EcYrbL (SEQ ID NO:56), PsMupP (SEQ ID NO:57), EcAppA (SEQ ID NO:58), EcGph (SEQ ID NO:59), EcSerB (SEQ ID NO:60), EcNagD (SEQ ID NO: 61), EcYbhA (SEQ ID NO:62), EcYbiV (SEQ ID NO:63), EcYbjL (SEQ ID NO:64), EcYfbR (SEQ ID NO:65), EcYicH (SEQ ID NO:66), EcYjgL (SEQ ID NO:67), EcYjjG (SEQ ID NO:68), EcYrfG (SEQ ID NO:69), EcYbiU (SEQ ID NO:70), ScDOG1 (SEQ ID NO:71) and BsAraL (SEQ ID NO:72) are expressed.

Yeast expression plasmid p2a_2μ_SL-glmS is based on p2a_2μ_sia but modified in a way that also KILAC12 (lactose permease from Kluyveromyces lactis), NmneuA (CMP-sialic acid synthase from Neisseria meningitides) and Pdbst (sialyltransferase Photobacterium damselae) are expressed.

Plasmids were maintained in the host E. coli DH5α (F⁻, phi80dlacZdeltaM15, delta(lacZYA-argF)U169, deoR, recA1, endA1, hsdR17 (rk⁻, mk⁺), phoA, supE44, lambda⁻, thi-1, gyrA96, relA1). Bought from Invitrogen.

Gene expression promoters: Genes are expressed using synthetic constitutive promoters, as described in by Blazeck (Biotech. and Bioeng., Vol. 109, No. 11, 2012).

Heterologous and homologous expression: Genes that needed to be expressed, be it from a plasmid or from the genome were synthetically synthetized with one of the following companies: DNA2.0, Gen9 or IDT

Expression could be further facilitated by optimizing the codon usage to the codon usage of the expression host. Genes were optimized using the tools of the supplier.

Cultivations conditions: In general, yeast strains were initially grown on SD CSM plates to obtain single colonies. These plates were grown for 2-3 days at 30° C.

Starting from a single colony, a preculture was grown over night in 5 mL at 30° C., shaking at 200 rpm. Subsequent 500 mL shake flask experiments were inoculated with 2% of this preculture, in 100 mL media. These shake flasks were incubated at 30° C. with an orbital shaking of 200 rpm. The use of an inducer is not required as all genes are constitutively expressed.

### Material and Methods B. subtilis

Media: Two different media are used, namely a rich Luria Broth (LB), a minimal medium for shake flask (MMsf). The minimal medium uses a trace element mix.

Trace element mix consisted of 0.735 g/L CaCl2.2H20, 0.1 g/L MnCl2.2H20, 0.033 g/L CuCl2.2H20, 0.06 g/L CoCl2.6H20, 0.17 g/L ZnCl2, XX g/L H3B04, XX g/L Na2EDTA·2H20 and 0.06 g/L Na2Mo04. The Fe-citrate solution contained 0.135 g/L FcCI3·6H20, 1 g/L Na-Citrate (Hoch 1973 PMC1212887).

The Luria Broth (LB) medium consisted of 1% tryptone peptone (Difco, Erembodegem, BE), 0.5% yeast extract (Difco) and 0.5% sodium chloride (VWR, Leuven, BE).

Luria Broth agar (LBA) plates consisted of the LB media, with 12 g/L agar (Difco, Erembodegem, BE) added.

Minimal medium for shake flask experiments (MMsf) contains 2 g/L (NH4) 2S04, 7.5 g/L KH₂P04, 17.5 g/L K2HP04, 1.25 g/L Na-Citrate, 0.25 g/L MgS04·7H20, 0.05 g/L tryptophan, from 10 up to 30 g/L glucose or another carbon source including but not limited to glucose, fructose, maltose, glycerol and maltotriose, 10 mL/L trace element mix, and 10 mL/L Fe-citrate solution. The medium was set to a pH of 7 with IM KOH.

Complex medium, e.g., LB, was sterilized by autoclaving (121° C., 21) and minimal medium (MMsf) by filtration (0.22 μm Sartorius). If necessary, the medium was made selective by adding an antibiotic (e.g., zeocin (20 mg/L)).

Strains: B. subtilis 168, available at Bacillus Genetic Stock Center (Ohio, USA).

### Plasmids and Gene Overexpression

Plasmids for gene deletion via Cre/lox are constructed as described by Yan et al. (Appl & environm microbial, sept 2008, p5556-5562).

Expression vectors can be found at Mobitec (Germany), or at ATCC (ATCC® number 87056). The genes BsglmS, ScGNA1 and CjneuB are cloned in these expression vectors. A suitable promoter for expression can be derived from the part repository (iGem): sequence id: BBa_K143012, BBa_K823000, BBa_K823002 or BBa_K823003. Cloning can be performed using Gibson Assembly, Golden Gate assembly, Cliva assembly, LCR or restriction ligation.

Plasmids are maintained in the host E. coli DH5α (F⁻, phi 80dlacZdeltaM15, delta(lacZYA-argF) U169, deoR, recA1, endA1, hsdR17 (rk⁻, mk⁺), phoA, supE44, lambda, thi-1, gyrA96, relA1). Bought from Invitrogen.

### Gene Disruptions

Disrupting of genes is done via homologous recombination with linear DNA and transformation via the electroporation as described by Xue et al. (J. microb. Meth. 34 (1999) 183-191). The method of gene knock-outs is described by Liu et al. (Metab. Engine. 24 (2014) 61-69). This method uses 1000 bp homologies up- and downstream of the target gene. The homologies to be used in this disclosure, are listed in table D. After the modification, the mutants are verified using primers upstream and downstream of the modified region. These primers are given in table E. Next, the modification is confirmed by sequencing (performed at LGC Genomics (LGC group, Germany)).

TABLE D

| Gene to be | Upstream | Downstream |
|---|---|---|
| disrupted | homology | homology |
| nagA-nagB | Gactgcaagatttcg | Aaggaacatgctgac |
| | gcctgggcggacggg | ttatgaatatcaata |
| | aatcgtcagttttgt | aacaatcgcctattc |
| | aatttctgtatcaat | cgatttactatcaga |
| | gattttcatggtctc | ttatggagcaattaa |
| | ttcctcaagtccgag | aaacccaaattaaga |
| | ccggtcgtattgctt | acggagagctgcagc |
| | gccctgctcccagag | cggatatgcctcttc |
| | ttcaagattcatgac | cttctgagcgcgaat |
| | aatcgtgattcgttt | atgccgaacaattcg |
| | attgcttctgaccgc | ggatcagccggatga |
| | gccagcgccaaatag | cagttcgccaggcgc |
| | cgtcatcacattgat | tttctaatttagtta |
| | aatgccaaggcccct | atgaaggcttgctct |
| | gatctcaagaaggtg | atcgcctgaaagggc |
| | ctcaattaattccgg | ggggcacctttgtca |
| | agcgtttcccacaag | gcaagccaaaaatgg |
| | agtatcctgatcctc | aacaagcacttcaag |
| | ctgccgtatttcaac | ggctgacaagcttta |
| | gcaatcatcggcaac | ccgaggatatgaaaa |
| | aaggcgatgccctct | gccgcgggatgacac |
| | tttcacaagctctag | cgggcagcaggctca |
| | cgctgtttcgctttt | ttgattatcagctta |
| | tccgacgccgctttt | ttgattcaactgagg |
| | tcctgtgatcagcac | agctcgcggctatat |
| | gccgacaccatatat | taggctgcgggcacc |
| | atcgacaagaacgcc | cctcctctatccata |
| | atgaattgctgtggt | aaatcactcgggtgc |
| | aggcgccagcctgct | ggctggcaaatgata |
| | ctcaaggaagttggt | ttccgatggcgattg |
| | taaacggcttgacag | agtcctcacatattc |
| | tcttgtcgttttcag | cgtttgagcttgcgg |
| | cggcgatctgaggac | gtgaattgaacgaat |
| | aggcaccccattttt | cgcattttcagtcgt |
| | ctcggaggcgtcaat | cgatctatgatcata |
| | cagctcctgcgggat | ttgaaaggtacaaca |
| | gggcatatctctaga | gcataccgatttccc |
| | aagaataatagctgg | gtgcaaaacaggagc |
| | tgttacatcagtgca | ttgagccaagcgctg |
| | cagagaatccattcg | ccaccacggaagaag |
| | ctgctttttctcctc | cgaatattcttggta |
| | ttcaggaagctgttc | ttcaaaagggagcgc |
| | aaagaaagaaagctc | ctgtcctattaatta |
| | tgtttttccgagaag | aacgaacaacatatc |
| | ctgcacgcgctccct | tgcagaacggaactg |
| | cgggtaatatgtaaa | cttttgagcatgcaa |
| | atatccggcaatttc | aatccgtatacagag |
| | aatacctggtcttga | gcgaccgttatacat |
| | taggtcactcattgt | ttgtccactatatgg |
| | aatcgggcggttaat | atcgtctttcataaa |
| | tccttcttctccgct | aaaagcctccaaccc |
| | gattaattccaaatt | tttttaaggattgga |
| | gaactgttccattac | gacatggcgaaaatc |
| | gtcttttgtgcgaac | aaactggtctggtgc |
| | ctttgccacgatatg | cggacgatatgtttc |
| | ttcctcctgttccgg | ttttttcgtcttgaa |
| | gctgccccgagcttg | cttccagatcggtga |
| | ctcacaatactttca | tttcgttttgccgtt |
| | ttttatcactttcgg | aaaactgtcttccac |
| | gcttgaacctaaaac | tataatgtaccaata |
| | agattttataaaagg | ataaacagactgcgg |
| | ggggaaaacacctca | ttcaagatgatccca |
| | gctggtctagatcac | gcggaattcagctgt |
| | tagtctgaaaaagag | gtccccgctcttcac |
| | taaaataaaggtatt | ttgctcccgttttcc |
| | caaattccagaaagg | gagctcttcattggt |
| | cggatcatct | atatacgtta |
| | (SEQ ID NO: 33) | (SEQ ID NO: 34) |
| gamA | Tggcggacatggaat | Gtgacaccccctcaa |
| | aaatcacaaacgaca | agagatagacaagca |
| | aagatgacgccggca | ccatatttgttatga |
| | agaatagagttaatc | ccaatttatgatact |
| | aaatagagcacgggc | tgtcattacgaattt |
| | gcaacgaacaagaaa | agcaccgcccttatc |
| | gaaaactcaaccggt | aaactgtcaatatta |
| | tctgtaattccggtc | atttctgaaaatttg |
| | agcatagatgtgagc | ttataaaagaaggat |
| | gccgcagaaatcatc | acaaatctttcatat |
| | acgccggagatcatt | tgggagggcaaatgg |
| | tttttcttttccgga | tattatggtctcaat |
| | cgcgcggtatggata | gaaaaagaacggatt |
| | atggcaagagcaacg | gcatacagaatgggg |
| | gccggcagacagaaa | agaatgaaatgacag |
| | atcatgtaagggaaa | ctttatattctgtta |
| | tcccccatcataaag | tcaagtttaaaatca |
| | cgcccggctgtcggg | ttgagttaattaaat |
| | tctcccgcgaaaaac | cgggcaaatatcagg |
| | cttgtcaggtcgccg | cgaatgatcagctgc |
| | gttacggtgttgcct | cgacggagagtgagt |
| | gttgatgggtctgtg | tttgcgaacaatatg |
| | tattctcccatcata | atgtcagcagaacaa |
| | aaatagaaaggcgta | ctgtgagactggctc |
| | taaaaaatatgatgc | tgcagcagctagagc |
| | aggccaaaaggaatc | ttgagggatatatta |
| | agcaaacgatagatc | aaagaattcaaggaa |
| | gttgcataaaagaac | aagggacatttgtat |
| | aggccgactgttgaa | cggcggccaaaatac |
| | tcggcaattaaactg | aaacgccgattccgc |
| | ctggctgcgttaatt | ataagattacgagct |
| | ccgttttggatcagc | ttgcagaacaaatga |
| | ggccaaacgaatgag | gaggacttcgttctg |
| | aaaatgacgccgatc | aatcaaaagtgcttg |
| | accaatgaactgacg | agcttgtggtgattc |
| | gaagtaatgatcggg | ctgccgatcattcca |
| | acaaagcgttttcca | tcgccgagcttttga |
| | gagaaaaatccaagg | aaatgaaagagaatg |
| | accggatgcagctcg | aacctgtcaacaagc |
| | attgatgaaaatcgc | ttgtcagagtcagat |
| | ttatataaataggcg | acgccgagggggaac |
| | gcgagaagcccgata | ctttgcagtatcata |
| | atgattcctccgaaa | cctcatatattccct |
| | acccccatatcaatc | ggaaggcggcaccgg |
| | aggtgctcggctcct | ggctggcgcaggagg |
| | tcatacggaggctga | aatgcaccggctcgc |
| | aggccgagtaatttt | tgtttgaattgttaa |
| | cccatattgtcgagg | ggacaaaatacaata |
| | gtgacggttaaaatt | ttgaaatcagcaggg |
| | aagtatccgatgaca | gcacggaatcgatcg |
| | gcggcaagtceggct | aaccgattttaacgg |
| | acaccttctccgccg | atgaaacgatcagcg |
| | gctaatccgatcgcg | gacacttattaacca |
| | acccccacggcgaaa | atgtcggagcgcctg |
| | atcagcggaaggtta | cgtttttatcagaat |
| | tcgaatacaacgccg | cccttacctatgata |
| | cccgcatcctttata | aaaatgaagaagtgg |
| | atagggatgttcagt | tggaatatgcgcaaa |
| | aaatccttgtctccg | ttattacacggggag |
| | aaacggagcaaaaga | accgaacgaaattca |
| | cctgctgccggcagg | ccgtagaacagtcat |
| | acggcaaccggagtc | atcattcataaagca |
| | atcaacgcgcggcca | atgtgttttaagaag |
| | agctgctgcagaatt | ggaatggtggttcta |
| | tgaaatgccttttta | tgtttttatttacga |
| | aacatgacagtctcc | atggaaaagtgctgt |
| | ttttattgtg | ggggagcagt |
| | (SEQ ID NO: 35) | (SEQ ID NO: 36) |

TABLE E

| Target | | |
|---|---|---|
| gene | Fw primer | Rv primer |
| nagA-nagB | Tgtaatcgggc | Gccctttcaggc |
| | ggttaattc | gatagag |
| | (SEQ ID NO: 37) | (SEQ ID NO: 38) |
| gamA | Acggcgaaaat | Tcactctccgtc |
| | cagcggaag | ggcagctg (SEQ |
| | (SEQ ID NO: 39) | ID NO: 40) |

### Heterologous and Homologous Expression

Genes that needed to be expressed, be it from a plasmid or from the genome were synthetically synthetized with one of the following companies: DNA2.0, Gen9 or IDT.

Expression could be further facilitated by optimizing the codon usage to the codon usage of the expression host. Gene were optimized using the tools of the supplier.

### Cultivations Conditions

A preculture, from a single colony on a LB-plate, in 5 mL LB medium was incubated for 8 h at 37° C. on an orbital shaker at 200 rpm. From this culture, 1 mL was transferred to 100 mL minimal medium (MMsf) in a 500 mL shake flask and incubated at 37° C. on an orbital shaker at 200 rpm. This setup is used for shake flask experiments. The use of an inducer is not required as all genes are constitutively expressed.

### Analytical Methods

Optical density: Cell density of the culture was frequently monitored by measuring optical density at 600 nm (Implen Nanophotometer NP80, Westburg, BE). Cell dry weight was obtained by centrifugation (10 min, 5000 g, Legend XIR Thermo Scientific, BE) of 20 g reactor broth in pre-dried and weighted falcons. The pellets were subsequently washed once with 20 mL physiological solution (9 g/L NaCI) and dried at 70° C. to a constant weight. To be able to convert OD₆ₒₒₙₘ measurements to biomass concentrations, a correlation curve of the OD₆ₒₒₙₘ to the biomass concentration was made.

Measurement of cell dry weight: From a broth sample, 4×10 g was transferred to centrifuge tubes, the cells were spun down (5000 g, 4° C., 5 min), and the cells were washed twice with 0.9% NaCI solution. The centrifuge tubes containing the cell pellets were dried in an oven at 70° C. for 48 h until constant weight. The cell dry weight was obtained gravimetrically; the tubes were cooled in a desiccator prior to weighing.

Liquid chromatography: The concentration of carbohydrates like, but not limited to, glucose, fructose and lactose were determined with a Waters Acquity UPLC H-class system with an ELSD detector, using a Acquity UPLC BEH amide, 130 Å, 1.7 μm, 2.1 mm×50 mm heated at 35° C., using a 75/25 acetonitrile/water solution with 0.2% triethylamine (0.130 mL/min) as mobile phase.

Sialyllactose was quantified on the same machine, with the same column. The eluent however was modified to 75/25 acetonitrile/water solution with 1% formic acid. The flow rate was set to 0.130 mL/min and the column temperature to 35° C.

Sialic acid was quantified on the same machine, using the REZEX ROA column (300×7.8 mm ID). The eluent is 0.08% acetic acid in water. The flow rate was set to 0.5 mL/min and the column temperature to 65° C. GlcNAc and ManNAc were also measured using this method.

Growth rate measurement: The maximal growth rate (u Max) was calculated based on the observed optical densities at 600 nm using the R package grofit.

### Example 2: Production of Sialic Acid in E. coli

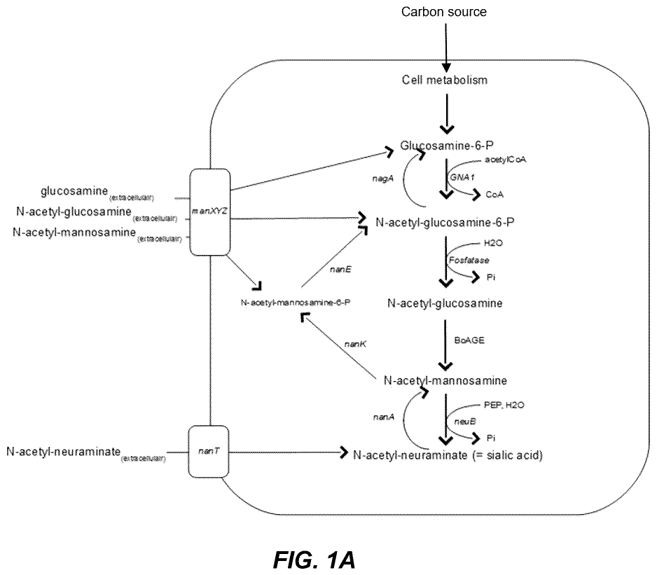

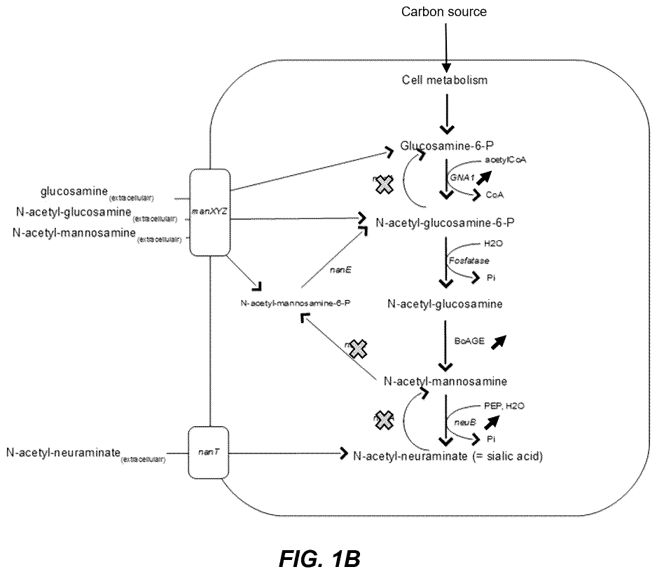

A first example provides an E. coli strain capable of producing N-acetylneuraminate (sialic acid) (see FIG. 1B).

A strain capable of accumulating glucosamine-6-phosphate using sucrose as a carbon source was further engineered to allow for N-acetylneuraminate production. The base strain overexpresses a sucrose phosphorylase from Bifidobacterium adolescentis (BaSP), a fructokinase from Zymomonas mobilis (Zmfrk), a mutant fructose-6-P-aminotransferase (EcglmS*54, as described by Deng et al. (Biochimie 88, 419-429 (2006))). To allow for gene sialic acid production the operons nagABCDE, nanATEK and manXYZ were disrupted. BaSP and Zmfrk were introduced at the location of nagABCDE and EcglmS*54 was introduced at the location of nanATEK. These modifications were done as described in Example 1 and are based on the principle of Datsenko & Wanner (PNAS USA 97, 6640-6645 (2000)).

In this strain, the biosynthetic pathway for producing sialic acid as described in this disclosure, was implemented by overexpressing a glucosamine-6-P-aminotransferase from S. cerevisiae (ScGNA1), a N-acetylglucosamine-2-epimerase from Bacteroides ovatus (BoAGE) and a sialic acid synthase from C. jejuni (CjneuB). ScGNA1 and BoAGE were expressed on locations nagABCDE and manXYZ, respectively. CjneuB was expressed using the high copy plasmid pCX-CjneuB.

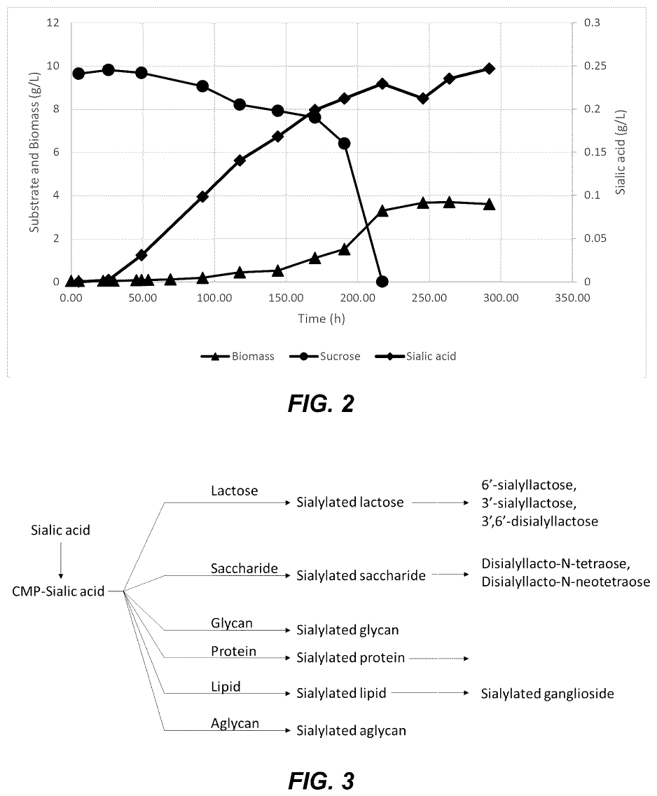

The strain was cultured as described in Example 1 (materials and methods). Briefly, a 5 mL LB preculture was inoculated and grown overnight at 37° C. This culture was used as inoculum in a shake flask experiment with 100 mL medium, which contains 10 g/L sucrose and was made as described in Example 1. Regular samples were taken and analyzed as described in Example 1. The evolutions of the concentrations of biomass, sucrose and sialic acid are easily followed and an end concentration of 0.22 g/L N-acetylneuraminate was produced extracellularly, as can be seen in FIG. 2.

The same organism also produces N-acetylneuraminate based on glucose, maltose or glycerol as carbon source.

### Example 3: Production of 6-Sialyllactose in E. coli

Use of the method and strains for producting 6-sialyllactose.

The strain of Example 3 is a daughter strain of the strain used in Example 2. The strain is further modified by overexpressing a lactose permease EclacY from E. coli (as described and demonstrated in Example 1 of WO 2016/075243, which is hereby incorporated by reference), a CMP-sialic acid synthethase from N. meningitides (NmneuA) and a sialyltransferase from Photobacterium damselae (Pdbst). On top of that lacZ is disrupted.

The genes NmneuA and Pdbst, are expressed from a plasmid, together with CjneuB. This plasmid is pCX-CjneuB-NmneuA-Pdbst, and is made as described in Example 1.

The strain is inoculated as a preculture consisting of 5 ml LB medium as described in Example 1. After growing overnight at 37° C. in an incubator. 1% of this preculture is inoculated in a shake flask containing 100 ml medium (MMsf) containing 10 g/l sucrose as carbon source and 10 g/l lactose as precursor. The strain is grown for 300h at 37° C.

This strain produces quantities of 6-sialyllactose.

### Example 4: Production of Sialic Acid in S. cerevisiae Using Heterologous Fructose-6-P-Aminotransferase

Use of an eukaryotic organism, in the form of S. cerevisiae. This method utilizing the pathway of the disclosure shall be obtained in S. cerevisiae by introducing and expressing a N-acetylglucosamine-2-epimerase (for example, from Bacteroides ovatus (BoAGE)) and a sialic acid synthase (for example, from C. jejuni (CjneuB)).

As starting point, a strain with increased metabolic flux toward N-acetylglucosamine-6-phosphate is needed. This is achieved by overexpressing the fructose-6-P-aminotransferase mutant from E. coli (EcglmS*54).

To create a N-acetylneuraminate producing S. cerevisiae according to this disclosure, the genes are introduced via a 2-micron plasmid (Chan 2013 (Plasmid 70 (2013) 2-17)) and the genes are expressed using synthetic constitutive promoters (Blazeck 2012 (Biotech. and Bioeng., Vol. 109, No. 11)) as also described in Example 1. The specific plasmid used in this embodiment is p2a_2μ_sia_glmS. This plasmid is introduced into Saccharomyces cerevisae using the transformation technique described by Gietz and Woods (2002, PMID 12073338) and a mutant strain is obtained.

The strain is capable of converting fructose-6-phosphate into glucosamine-6-phosphate, followed by glucosamine-6-phosphate conversion in N-acetylglucosamine-6-phosphate. This N-acetylglucosamine-6-phosphate moiety is further converted to N-acetylglucosamine, the N-acetylglucosamine into N-acetylmannosamine and finally this N-acetylmannosamine is converted into N-acetylneuraminate.

A preculture of the strain is made in 5 mL of the synthetic defined medium SD-CSM containing 22 g/L glucose and grown at 30° C. as described in Example 1. This preculture is inoculated in 100 mL medium in a shakeflask with 10 g/L sucrose as sole carbon source and grown at 30° C. Regular samples are taken and the production of N-acetylneuraminate is measured as described in Example 1. This strain and method produces quantities of N-acetylneuraminate.

The same organism also produces N-acetylneuraminate based on glucose, maltose or glycerol as carbon source.

### Example 5: Production of 6-Sialyllactose in S. cerevisiae

Use of a eukaryote, in the form of Saccharomyces cerevisae. This method utilizing the pathway of the disclosure shall be obtained in S. cerevisiae by introducing and expressing a N-acetylglucosamine-2-epimerase (for example, from Bacteroides ovatus (BoAGE)) and a sialic acid synthase (for example, from C. jejuni (CjneuB)).

On top of that, further modifications are made in order to produce 6sialyllactose. These modifications comprise the addition of a lactose permease, a CMP-sialic acid synthase and a sialyltransferase. The preferred lactose permease is the KILAC12 gene from Kluyveromyces lactis (WO 2016/075243). The preferred CMP-sialic acid synthase and the sialyltransferase are, respectively, NmneuA from N. meningitides and Pdbst from Photobacterium damselae, as also described in Example 3.

As starting point, a strain with increased metabolic flux toward N-acetylglucosamine-6-phosphate is needed. This is achieved by overexpressing the fructose-6-P-aminotransferase mutant from E. coli (EcglmS*54).

To create a N-acetylneuraminate producing S. cerevisiae according to this disclosure, the genes are introduced via a 2-micron plasmid (Chan 2013 (Plasmid 70 (2013) 2-17)) and the genes are expressed using synthetic constitutive promoters (Blazeck (2012)) as also described in Example 1. The specific plasmid used in this embodiment is p2a_2μ_sia_glmS. This plasmid is introduced into S. cerevisiae using the transformation technique described by Gietz and Woods (2002) and a mutant strain is obtained.

The strain is capable of converting fructose-6-phosphate into glucosamine-6-phosphate, the glucosamine-6-phosphate into N-acetylglucosamine-6-phosphate, the N-acetylglucosamine-6-phosphate into N-acetylglucosamine, the N-acetylglucosamine into N-acetylmannosamine and finally the N-acetylmannosamine into N-acetylneuraminate. The N-acetylmannosamine is then converted to CMP-sialic acid and transferred to lactose to obtain 6sialyllactose.

A preculture of the strain is made in 5 mL of the synthetic defined medium SD-CSM containing 22 g/L glucose and grown at 30° C. as described in Example 1. This preculture is inoculated in 100 mL medium in a shakeflask with 10 g/L sucrose as sole carbon source and grown at 30° C. Regular samples are taken and the production of N-acetylneuraminate is measured as described in Example 1. This strain and method produces quantities of 6sialyllactose.

The same organism also produces N-acetylneuraminate based on glucose, maltose or glycerol as carbon source.

### Example 6: Production of Sialic Acid in S. cerevisiae Using Autologous Fructose-6-P-Aminotransferase

Use of a eukaryote in the form of Saccharomyces cerevisae. This method utilizing the pathway of the disclosure shall be obtained in S. cerevisiae by introducing and expressing a N-acetylglucosamine-2-epimerase (for example, from Bacteroides ovatus (BoAGE)) and a sialic acid synthase (for example, from C. jejuni (CjneuB)).

As starting point, a strain with increased metabolic flux toward N-acetylglucosamine-6-phosphate is needed. This is achieved by overexpressing the native fructose-6-P-aminotransferase ScGFA1.

To create a N-acetylneuraminate producing S. cerevisiae according to this disclosure, the genes are introduced via a 2-micron plasmid (Chan (2013) and the genes are expressed using synthetic constitutive promoters (Blazeck (2012) as also described in Example 1. The specific plasmid used in this embodiment is p2a_2μ_sia_GFA1. This plasmid is introduced into Saccharomyces cerevisae using the transformation technique described by Gietz and Woods (2002) and a mutant strain is obtained.

The strain is capable of converting fructose-6-phosphate into glucosamine-6-phosphate, the glucosamine-6-phosphate into N-acetylglucosamine-6-phosphate, the N-acetylglucosamine-6-phosphate into N-acetylglucosamine, the N-acetylglucosamine into N-acetylmannosamine and finally the N-acetylmannosamine into N-acetylneuraminate.

A preculture of the strain is made in 5 mL of the synthetic defined medium SD-CSM containing 22 g/L glucose and grown at 30° C. as described in Example 1. This preculture is inoculated in 100 mL medium in a shakeflask with 10 g/L sucrose as sole carbon source and grown at 30° C. Regular samples are taken and the production of N-acetylneuraminate is measured as described in Example 1. This strain and method produces quantities of N-acetylneuraminate.

The same organism also produces N-acetylneuraminate based on glucose, maltose or glycerol as carbon source.

### Example 7: Production of Sialyllactoses and Other Sialylated Compounds

In an alternative embodiment of Example 3, the sialyltransferase is changed to another sialyltransferase with different activity. This can be an α-2,3-sialyltransferase α-2,6-sialyltransferase, an α-2,8-sialyltransferase or a combination thereof. These sialyltransferases are widely available in nature and well annotated.

In this way, production of different sialyllactoses like, e.g., 6-sialyllactose, 3-sialyllactose or a mixture thereof can be obtained.

The strains are cultivated as stated in Example 1 and Example 3.

The pathways created in Examples 2 to 7 can also be combined with other pathways for the synthesis of larger oligosaccharides, e.g., sialyl-lacto-N-triose, sialyllacto-N-tetraose, disialyllactose-N-tetraose, sialyllacto-N-neotetraose, and disialyllactose-N-neotetraose. To this end, the transferases to synthetize these glycosidic bonds are co-expressed with the pathway genes to form CMP-sialic acid and the transferase (as described above) to sialylate the oligosaccharide.

Examples of such sialyltransferases are ST6GalI, ST6GalII, ST3GalI until VI, ST6GalNAc I until VI and ST8Sia I until VI, as described by Datta (Current Drug Targets, 2009, 10, 483-498) and Harduin-Lepers (Biochimie 83 (2001) 727-737). Further examples originating from marine organisms are described by Yamamoto (Mar. Drugs 2010, 8, 2781-2794).

### Example 8: Production of Sialylated Lacto-N-Neotetraose

This experiment demonstrates the functionality of the disclosure of the production of other sialylated oligosaccharides, e.g., sialyltated lacto-N-neotetraose.

A lacto-N-neotetraose producing strain was developed following the protocol described in Example 1. For production, the expression of a N-acetylglucosaminyltransferase and a galactosyltransferase are needed, this is achieved by introduction of the genes NmlgtA and NmlgtB, respectively, both from N. meningitides. Next, the lactose importer EclacY from E. coli is (as described and demonstrated in Example 1 of the incorporated WO 2016/075243). Finally, the genes ushA and galT are knocked out. In this way, a lacto-N-neotetraose producing strain is obtained.

To be able to grow on lactose and produce N-acetylglucosamine-6-phosphate, a sucrose phosphorylase from Bifidobacterium adolescentis (BaSP), a fructokinase from Zymomonas mobilis (frk) and a mutant fructose-6-P-aminotransferase (EcglmS*54, as described by Deng et al. (Biochimie 88, 419-429 (2006))) were overexpressed as described in Example 1.

In this strain, the method for producing sialic acid as described in this disclosure, was implemented by overexpressing a glucosamine-6-P-aminotransferase from S. cerevisiae (ScGNA1), a N-acetylglucosamine-2-epimerase from Bacteroides ovatus (BoAGE) and a sialic acid synthase from C. jejuni (CjneuB). ScGNA1 and BoAGE are expressed on locations nagABCDE and manXYZ, respectively. CjneuB is expressed from plasmid pCX-CjneuB-NmneuA-Pdbst.

Sialylation of the lacto-N-neotetraose moiety is performed by the conversion of sialic acid to CMP-salic acid by a CMP sialic acid synthethase, e.g., NmneuA from Neisseria meningtides, subsequently followed by a sialyl transferase, e.g., Pdbst, from Photobacterium damselae. These genes (NmneuA and Pdbst) are expressed from the high copy plasmid pCX-CjneuB-NmneuA-Pdbst.

The strain is cultured as described in Example 1 (materials and methods). Briefly, a 5 mL LB preculture is inoculated and grown overnight at 37° C. This culture was used as inoculum in a shake flask experiment with 100 mL medium, which contains 10 g/L sucrose as carbon and energy source, 10 g/L lactose as precursor and was made according to the description in Example 1. Regular samples are taken and analyzed. This strain produces quantities of sialylated lacto-N-neotetraose.

Alternative glycosyltransferases are possible. If EcWgbO (from E. coli 055: H7) is expressed instead of NmlgtB, e.g., production of sialylated lacto-N-tetraose is obtained.

### Example 9: Production of Sialic Acid with B. subtilis

In another embodiment, this disclosure can be used for production of N-acetylneuraminate in B. subtilis, yet another bacterial production host.

A N-acetylneuraminate producing strain is obtained through this disclosure by starting with a strain, capable of overproducing glucosamine-6-phosphate intracellularly. For this, the native fructose-6-P-aminotransferase (BsglmS) is overexpressed. The following enzymatic activities are disrupted by knocking out the genes nagA, nagB and gamA: N-acetylglucosamine-6-phosphate deacetylase and glucosamine-6-phosphate isomerase.

In this strain, the method for producing sialic acid as described in this disclosure, is implemented by overexpressing a glucosamine-6-P-aminotransferase from S. cerevisiae (ScGNA1), a N-acetylglucosamine-2-epimerase from Bacteroides ovatus (BoAGE) and a sialic acid synthase from C. jejuni (CjneuB). These genes are introduced via a plasmid, as described in Example 1.

The strain is cultured as described in Example 1 (Materials and Methods). Briefly, a 5 mL LB preculture is inoculated and grown overnight at 30° C. This culture is used as inoculum in a shake flask experiment with 100 mL medium, which contains 10 g/L sucrose and is made according to the description in Example 1. This strain produces quantities of N-acetylneuraminic acid.

### Example 10: Fermentations of 6-Sialyllactose Producing Strain with No Excretion of GlcNAc, ManNAc or Sialic Acid

Another example is use of the method and strains for producing 6-sialyllactose.

An E. coli strain capable of accumulating glucosamine-6-phosphate using sucrose as a carbon source was further engineered to allow for N-acetylneuraminate production. This base strain overexpresses a sucrose phosphorylase from Bifidobacterium adolescentis (BaSP), a fructokinase from Zymomonas mobilis (Zmfrk), a mutant fructose-6-P-aminotransferase (EcglmS*54, as described by Deng et al. (Biochimie 88, 419-429 (2006)). To allow for 6-sialyllactose production the operons nagABCDE, nanATEK and manXYZ were disrupted. BaSP and Zmfrk were introduced at the location of nagABCDE, EcglmS*54 was introduced at the location of nanATEK. These modifications were done as described in Example 1 and are based on the principle of Datsenko & Wanner (PNAS USA 97, 6640-6645 (2000)).

In this strain, the biosynthetic pathway for producing 6-sialyllactose as described in this disclosure, was implemented by overexpressing a glucosamine-6-P-aminotransferase from S. cerevisiae (ScGNA1), a N-acetylglucosamine-2-epimerase from Bacteroides ovatus (BoAGE) and a sialic acid synthase from N. meningitides (NmneuB). ScGNA1 and BoAGE were expressed on locations nagABCDE and manXYZ, respectively. NmNeuB was expressed using the high copy plasmid pBR322-NmNeuB. The strain is further modified by overexpressing a lactose permease EclacY from E. coli (as described and demonstrated in Example 1 of the incorporated WO 2016/075243), a CMP-sialic acid synthethase from N. meningitides (NmNeuA) and a sialyltransferase from Photobacterium damselae (Pdbst). On top of that, lacZ is disrupted. NmNeuA and Pabst were expressed using the low copy plasmid pSC101-NmneuA-Pdbst.

The strain was cultured in a bioreactor as described in Example 1 (materials and methods). Briefly, a 5 mL LB preculture was inoculated and grown overnight at 37° C. This culture was used as inoculum in a shake flask experiment with 500 mL medium, which contains 10 g/L sucrose and was made as described in Example 1. This culture was used as inoculum in a 2 L bioreactor experiment. Regular samples were taken and analyzed as described in Example 1. The final concentration of 6-sialyllactose was 30.5 g/L. No extracellular GlcNAc, ManNAc and sialic acid was detected during the fermentation and in the final broth.

The same organism also produces 6-sialyllactose based on glucose, maltose or glycerol as carbon source.

### Example 11: Effect of Phosphatase on Growth and Production of Sialic Acid

A further example provides growth results and sialic acid production of several E. coli strains capable of producing N-acetylneuraminate (sialic acid) wherein the strains are expressing an extra phosphatase as indicated hereunder.

The base strain overexpresses a mutant fructose-6-P-aminotransferase (EcglmS*54, as described by Deng et al. (Biochimie 88, 419-429 (2006)), a glucosamine-6-P-aminotransferase from S. cerevisiae (ScGNA1), a N-acetylglucosamine-2-epimerase from Bacteroides ovatus (BoAGE) and a sialic acid synthase from C. jejuni (CjneuB). To allow for gene sialic acid production the operons nagABCDE and nanATEK. The lac YZA operon was replaced by only a single gene operon, the native lacY, which is required for the production of sialyllactose as described in Example 10. These modifications were done as described in Example 1 and are based on the principle of Datsenko & Wanner (PNAS USA 97, 6640-6645(2000)).

This base strain was then supplemented with different phosphatase bearing plasmids for comparing the effect of the phosphatase on growth and sialic acid production. The base strain was used as blank in the comparison. These plasmids consisted of, besides the phosphatase and a promoter driving expression of the phosphatase, a pSC101 ori and a spectomycin resistance marker. The following phosphatases were expressed: EcAphA (SEQ ID NO: 42), EcCof (SEQ ID NO:43), EcHisB (SEQ ID NO:44), EcOtsB (SEQ ID NO:45), EcSurE (SEQ ID NO:46), EcYacd (SEQ ID NO:47), EcYcjU (SEQ ID NO:48), EcYedP (SEQ ID NO:49), EcYfbT (SEQ ID NO:50), EcYidA (SEQ ID NO:51), EcYigB (SEQ ID NO:52), EcYihX (SEQ ID NO: 53), EcYniC (SEQ ID NO:54), EcYqaB (SEQ ID NO:55), EcYrbL (SEQ ID NO:56) and PsMupP (SEQ ID NO:57). Other phosphatases that are expressed are EcAppA (SEQ ID NO:58), EcGph (SEQ ID NO:59), EcSerB (SEQ ID NO:60), EcNagD (SEQ ID NO:61), EcYbhA (SEQ ID NO: 62), EcYbiV (SEQ ID NO:63), EcYbjL (SEQ ID NO:64), EcYfbR (SEQ ID NO:65), EcYicH (SEQ ID NO:66), EcYjgL (SEQ ID NO:67), EcYjjG (SEQ ID NO:68), EcYrfG (SEQ ID NO:69), EcYbiU (SEQ ID NO:70), ScDOG1 (SEQ ID NO:71) and BsAraL (SEQ ID NO:72).

In a first experiment a subset of the above described strains was used. In a second experiment a second subset of the above described strains were tested.

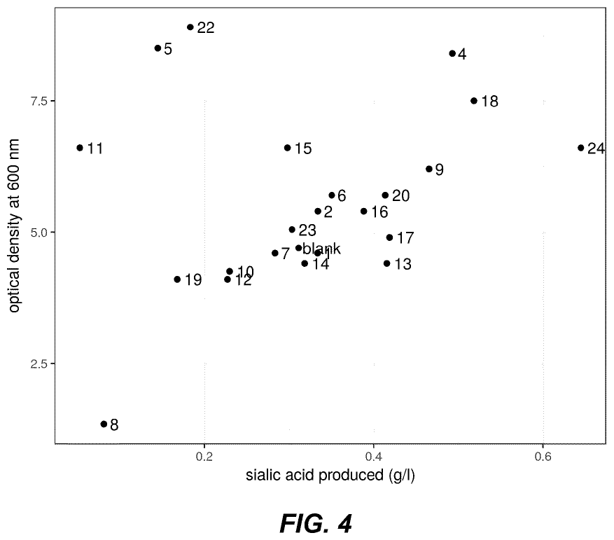

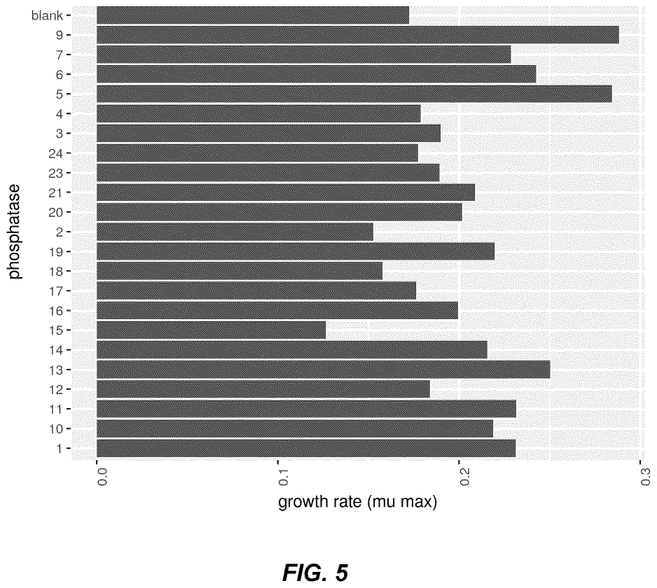

Each strain was cultured as described in Example 1 (materials and methods). Briefly, the workflow consists of 3 growth steps: first growth on LB, followed by growth on MMsf with 15 g/L glycerol, and finally a growth stage using 15 g/L glycerol MMsf. The first step is performed in a 96well plate, using 175 μL LB per well, and incubated overnight at 37° C. The second step is performed in a 96well plate using 175 μL medium, incubated for 24 h at 37° C. The final growth step was performed in: i) in a 96well plate using 175 μL medium, incubated at 37° C. to determine the u Max for the first experiment (see FIG. 5) and ii) in a 24well deepwell plates using 3 mL do determine sialic acid production and optical densities for the second experiment (see FIG. 4).

### Reference Table for FIGS. 4 and 5

| label | phosphatase | SEQ ID NO | Promotor |
|---|---|---|---|
| blank | NA | NA | NA |
| 1 | EcAphA | 42 | apFAB346 |
| 2 | EcAphA | 42 | apFAB87 |
| 3 | EcCof | 43 | apFAB87 |
| 4 | EcCof | 43 | apFAB346 |
| 5 | EcHisB | 44 | apFAB346 |
| 6 | EcOtsB | 45 | apFAB346 |
| 7 | EcSurE | 46 | apFAB346 |
| 8 | EcSurE | 46 | apFAB87 |
| 9 | EcYaed | 47 | apFAB346 |
| 10 | EcYaed | 47 | apFAB87 |
| 11 | EcYcjU | 48 | apFAB87 |
| 12 | EcYedP | 49 | apFAB87 |
| 13 | EcYfbT | 50 | apFAB87 |
| 14 | EcYidA | 51 | apFAB346 |
| 15 | EcYidA | 51 | apFAB87 |
| 16 | EcYigB | 52 | apFAB346 |
| 17 | EcYihX | 53 | apFAB346 |
| 18 | EcYihX | 53 | apFAB87 |
| 19 | EcYniC | 54 | apFAB346 |
| 20 | EcYniC | 54 | apFAB87 |
| 21 | EcYqaB | 55 | apFAB87 |
| 22 | EcYqaB | 55 | apFAB346 |
| 23 | EcYrbL | 56 | apFAB87 |
| 24 | PsMupP | 57 | apFAB87 |

Based on FIGS. 4 and 5 phosphatases enabling strains to grow better than the blank strain (no crippled growth) and producing more sialic acid than the blank strain, can be chosen.

Based on the above, it was found that phosphatases comprising at least Motif 1 and Motif 2 provide a strain that is not crippled and produces more sialic acid than the blank strain.

### Example 12: Identification of Further Sequences Related to the Phosphatases Used in the Methods of the Disclosure

Sequences (polypeptides) related to SEQ ID NOs: 43, 44, 45, 47, 48, 49, 50, 51, 52, 54, 55 and 57 were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical amino acids between the two compared polypeptide sequences over a particular length. In some instances, the default parameters may be adjusted to modify the stringency of the search. For example, the E-value may be increased to show less stringent matches. This way, short nearly exact matches may be identified.

Table 1A to 1K provides a list of homologue polypeptide sequences related to SEQ ID NO:43, 44, 45, 47, 48, 50, 51, 52, 54, 55 and 57, respectively.

TABLE 1A
Examples of polypeptides related to Ec Cof (SEQ ID
NO: 43), showing sequence identity to SEQ ID 43:

| % identity | | SEQ ID |
|---|---|---|
| (matgat) | short GenBank identifier | NO |
| 99.6 | Shigella flexneri WP_095762248.1 | 78 |
| 99.3 | Shigella boydii WP_095785299.1 | 79 |
| 98.2 | Escherichia fergusonii WP_024256925.1 | 80 |
| 89.3 | Staphylococcus aureus WP_094409981.1 | 81 |
| 89 | Escherichia albertii WP_000113024.1 | 82 |
| 81.6 | Citrobacter amalonaticus WP_046476411.1 | 83 |
| 81.6 | Salmonella enterica WP_023234244.1 | 84 |
| 80.5 | Escherichia coli WP_088543831.1 | 85 |

TABLE 1B
Examples of polypeptides related to Ec HisB (SEQ ID
NO: 44), showing sequence identity to SEQ ID 44:

| % identity | | SEQ ID |
|---|---|---|
| (matgat) | short GenBank identifier | NO |
| 99.4 | Shigella flexneri K-315 EIQ21345.1 | 86 |
| 99.2 | Escherichia albertii WP_059217413.1 | 87 |
| 98.9 | Shigella flexneri WP_094085559.1 | 88 |
| 98.6 | Shigella sonnei WP_077125326.1 | 89 |
| 98.6 | Escherichia coli WP_088129012.1 | 90 |
| 98 | Shigella dysenteriae WP_000080078.1 | 91 |
| 98 | Escherichia marmotae WP_038355110.1 | 92 |
| 94.6 | Salmonella bongori WP_000080052.1 | 93 |

TABLE 1C
Examples of polypeptides related to Ec OtsB (SEQ ID
NO: 45), showing sequence identity to SEQ ID 45:

| % identity | | SEQ ID |
|---|---|---|
| (matgat) | short GenBank identifier | NO |
| 99.6 | Shigella sonnei WP_077124555.1 | 94 |
| 99.6 | Escherichia coli WP_032172688.1 | 95 |
| 99.2 | Shigella flexneri WP_064198868.1 | 96 |
| 85.7 | Escherichia albertii WP_059227241.1 | 97 |
| 83.1 | Escherichia fergusonii WP_000165652.1 | 98 |

TABLE 1D
Examples of polypeptides related to Ec Yaed (SEQ ID
NO: 47), showing sequence identity to SEQ ID 47:

| % identity | | SEQ ID |
|---|---|---|
| (matgat) | short GenBank identifier | NO |
| 99.5 | Escherichia fergusonii WP_001140180.1 | 99 |
| 99.5 | Shigella sonnei WP_047565591.1 | 100 |
| 99 | Escherichia coli WP_061103769.1 | 101 |
| 95.8 | Escherichia albertii WP_001140171.1 | 102 |
| 93.2 | Kluyvera intermedia WP_047371746.1 | 103 |
| 93.2 | Citrobacter koseri WP_047458784.1 | 104 |
| 89 | Kosakonia arachidis WP_090122712.1 | 105 |
| 85.9 | Kluyvera cryocrescensWP_061282459.1 | 106 |
| 85.9 | Leclercia adecarboxylata WP_039030283.1 | 107 |

TABLE 1E
Examples of polypeptides related to Ec YcjUB (SEQ ID
NO: 48), showing sequence identity to SEQ ID NO: 48:

| % identity | | |
|---|---|---|
| (matgat) | short GenBank identifier | SEQ ID NO |
| 99.5 | Shigella sonnei WP_094313132.1 | 108 |
| 97.7 | Escherichia coli WP_000775764.1 | 109 |
| 95.4 | Escherichia coli WP_032302947.1 | 110 |
| 92.7 | Shigella flexneri OUZ88260.1 | 111 |

TABLE 1F
Examples of polypeptides related to Ec YfbT (SEQ ID
NO: 50), showing sequence identity to SEQ ID NO: 50:

| % identity | | SEQ ID |
|---|---|---|
| (matgat) | short GenBank identifier | NO |
| 99.1 | Shigella sonnei WP_094323443.1 | 112 |
| 87.5 | Citrobacter werkmanii NBRC 105721 | 113 |
| | GAL43238.1 | |
| 86.6 | Citrobacter freundii KGZ33467.1 | 114 |
| 86.6 | Citrobacter amalonaticus Y19 AKE59306.1 | 115 |
| 85.6 | Salmonella enterica WP_080095242.1 | 116 |
| 85.6 | Escherichia fergusonii WP_001203376.1 | 117 |
| 85.6 | Salmonella enterica subsp. enterica serovar | 118 |
| | Hadar KKD79316.1 | |

TABLE 1G
Examples of polypeptides related to Ec YidA (SEQ ID
NO: 51), showing sequence identity to SEQ ID NO: 51:

| % identity | | SEQ ID |
|---|---|---|
| (matgat) | short GenBank identifier | NO |
| 99.6 | Escherichia coli WP_053263719.1 | 119 |
| 99.3 | Escherichia fergusonii WP_000985562.1 | 120 |
| 99.3 | Shigella sonnei WP_094337696.1 | 121 |
| 94.4 | Trabulsiella guamensis WP_038161262.1 | 122 |
| 94.1 | Citrobacter amalonaticus WP_061075826.1 | 123 |
| 93.7 | Klebsiella pneumoniae WP_048288968.1 | 124 |
| 93.3 | Trabulsiella odontotermitis WP_054178096.1 | 125 |
| 90 | Enterobacter kobei WP_088221256.1 | 126 |

TABLE 1H
Examples of polypeptides related to Ec YigB (SEQ ID
NO: 52), showing sequence identity to SEQ ID NO: 52:

| % identity | | SEQ ID |
|---|---|---|
| (matgat) | short GenBank identifier | NO |
| 99.6 | Shigella sonnei WP_094322240.1 | 127 |
| 93.7 | Shigella sonnei WP_052962467.1 | 128 |
| 87 | Salmonella enterica WP_079797638.1 | 129 |
| 85.7 | Citrobacter braakii WP_080625916.1 | 130 |
| 81.9 | Enterobacter hormaechei WP_047737367.1 | 131 |
| 81.1 | Lelliottia amnigena WP_059180726.1 | 132 |
| 80.3 | Leclercia adecarboxylata WP_039031210.1 | 133 |

TABLE 1I
Examples of polypeptides related to Ec YniC (SEQ ID
NO: 54), showing sequence identity to SEQ ID NO: 54:

| % identity | | SEQ ID |
|---|---|---|
| (matgat) | short GenBank identifier | NO |
| 85.6 | Shigella flexneri 1235-66 EIQ75633.1 | 134 |
| 85.1 | Kosakonia sacchari WP_074780431.1 | 135 |
| 85.1 | Enterobacter mori WP_089599104.1 | 136 |
| 84.7 | Lelliottia amnigena WP_064325804.1 | 137 |
| 84.7 | Enterobacter sp. 638 WP_012017112.1 | 138 |
| 84.2 | Kosakonia radicincitans WP_071920671.1 | 139 |
| 84.2 | Salmonella enterica subsp. enterica serovar | 140 |
| | Newport str. CDC 2010K-2159 AKD18194.1 | |

TABLE 1J
Examples of polypeptides related to Ec YqaB (SEQ ID
NO: 55), showing sequence identity to SEQ ID NO: 55:

| % identity | | SEQ ID |
|---|---|---|
| (matgat) | short GenBank identifier | NO |
| 97.9 | Shigella flexneri K-315 EIQ18779.1 | 141 |
| 93.6 | Escherichia albertii WP_059215906.1 | 142 |
| 88.3 | Salmonella enterica WP_079949947.1 | 143 |
| 85.6 | Kluyvera intermedia WP_085006827.1 | 144 |
| 85.1 | Trabulsiella odontotermitis WP_054177678.1 | 145 |
| 84.6 | Yokenella regensburgei WP_006817298.1 | 146 |
| 84.6 | Raoultella terrigena WP_045857711.1 | 147 |
| 83.5 | Klebsiella pneumoniae WP_064190334.1 | 148 |

TABLE 1K
Examples of polypeptides related to Ps MupP (SEQ ID
NO: 57), showing sequence identity to SEQ ID NO: 57:

| % identity | | SEQ ID |
|---|---|---|
| (matgat) | short GenBank identifier | NO |
| 94.6 | Pseudomonas putida group WP_062573193.1 | 149 |
| 94.6 | Pseudomonas sp. GM84 WP_008090372.1 | 150 |
| 93.3 | Pseudomonas entomophila | 151 |
| 92.4 | Pseudomonas vranovensis WP_028943668.1 | 152 |
| 83.9 | Pseudomonas cannabina WP_055000929.1 | 153 |
| 93.3 | Pseudomonas monteilii WP_060480519.1 | 154 |

Sequences have been tentatively assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. Special nucleic acid sequence databases have been created for particular organisms, such as by the Joint Genome Institute.

### Example 13: Identification of Domains and Motifs Comprised in Polypeptide Sequences Useful in Performing the Methods of the Disclosure

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

The results of the InterPro scan of the polypeptide sequences as represented by SEQ ID NOs: 43, 44, 45, 47, 48, 49, 50, 51, 52, 54 and 55 are presented in Table 2.

TABLE 2
InterPro scan results (major accession numbers) of
the polypeptide sequence as represented by SEQ ID
NOs: 43, 44, 45, 47, 48, 49, 50, 51, 52, 54 and 55.

| Database | Accession number | Accession name |
|---|---|---|
| Interpro | IPR023214 | HAD superfamily |

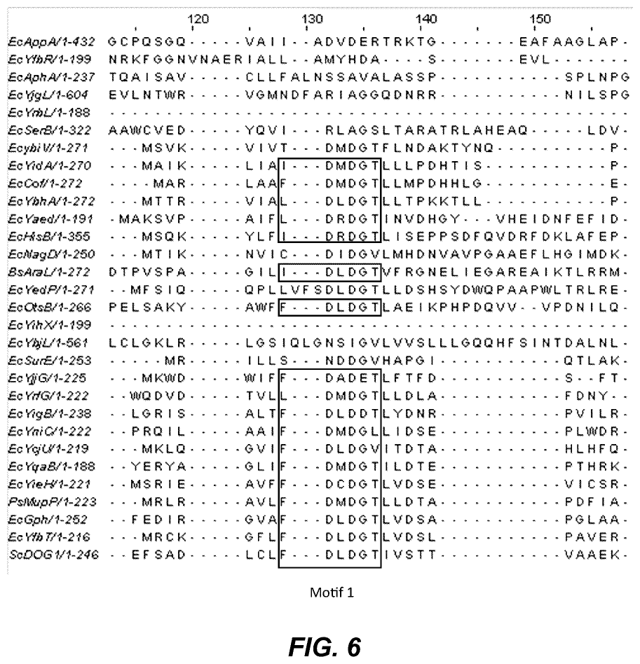

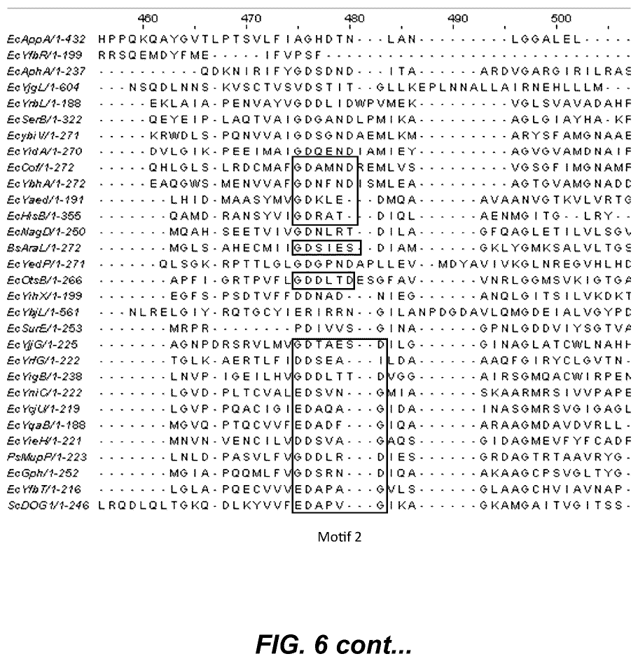

Alignment of the tested phosphatase polypeptides was done and FIG. 6 shows part of the alignment. Motif 1 and motif 2 are indicated with boxes. Alignment was made using clustalomega.

### Example 14: Effect of Phosphatase on Growth and Production of Sialic Acid in S. cerevisiae

A further example of sialic acid production of several Saccharomyces cerevisiae strains capable of producing N-acetylneuraminate (sialic acid) wherein the strains are expressing an extra phosphatase as indicated hereunder.

The strain used here is derived from the strain described in example 4. To enhance growth and production of sialic acid in S. cerevisiae according to this disclosure, the phosphatase genes are introduced via a 2-micron plasmid (Chan 2013 (Plasmid 70 (2013) 2-17)) and the genes are expressed using synthetic constitutive promoters (Blazeck 2012 (Biotechnology and Bioengineering, Vol. 109, No. 11)) as also described in Example 1. The specific plasmids used in this embodiment is p2a_2μ_sia_glmS-phospha. This plasmid based on the plasmid p2a_2μ_sia_glmS plasmid is described in Example 1. It is introduced into Saccharomyces cerevisae using the transformation technique described by Gietz and Woods (2002, PMID 12073338) and a mutant strain is obtained. The effect of phosphatase expression on growth and production of sialic acid of these mutants are evaluated as described in Example 11.

### Example 15: Effect of Phosphatase on Growth and Production of Sialic Acid in B. subtilis

In another embodiment, this disclosure can be used to enhance growth and production of sialic acid in B. subtilis, yet another bacterial production host.

The strain used here is derived from the strain described in example 9. Additionally to the alterations described in example 9, phosphatase genes EcAphA (SEQ ID NO: 42), EcCof (SEQ ID NO:43), EcHisB (SEQ ID NO:44), EcOtsB (SEQ ID NO:45), EcSurE (SEQ ID NO:46), EcYaed (SEQ ID NO:47), EcYcjU (SEQ ID NO:48), EcYedP (SEQ ID NO:49), EcYfbT (SEQ ID NO:50), EcYidA (SEQ ID NO:51), EcYigB (SEQ ID NO:52), EcYihX (SEQ ID NO: 53), EcYniC (SEQ ID NO:54), EcYqaB (SEQ ID NO:55), EcYrbL (SEQ ID NO:56), PsMupP (SEQ ID NO:57), EcAppA (SEQ ID NO:58), EcGph (SEQ ID NO:59), EcSerB (SEQ ID NO: 60), EcNagD (SEQ ID NO:61), EcYbhA (SEQ ID NO:62), EcYbiV (SEQ ID NO:63), EcYbjL (SEQ ID NO:64), EcYfbR (SEQ ID NO:65), EcYieH (SEQ ID NO:66), EcYjgL (SEQ ID NO:67), EcYjjG (SEQ ID NO:68), EcYrfG (SEQ ID NO:69), EcYbiU (SEQ ID NO:70), ScDOG1 (SEQ ID NO: 71) and BsAraL (SEQ ID NO:72) are overexpressed on a plasmid, as described in Example 1. Subsequently, this plasmid is introduced in B. subtilis. The effect of phosphatase expression on growth and production of sialic acid of the created mutants are evaluated as described in Example 11.

1.-18. (canceled)

19. A microorganism that produces at least one sialylated compound, wherein the microorganism;
i) comprises a sialic acid biosynthesis pathway that converts:
glucosamine-6-phosphate to N-acetylglucosamine-6-phosphate,
N-acetylglucosamine-6-phosphate to N-acetylglucosamine,
N-acetylglucosamine to N-acetylmannosamine, and
N-acetylmannosamine to N-acetyl-neuraminate,
ii) converts N-acetylneuraminate to cytidine monophosphate (CMP)-N-acetylneuraminate (also known as CMP-sialic acid), and
iii) comprises a heterologous sialyltransferase.

20.-23. (canceled)

24. The microorganism of claim 19, wherein the microorganism comprises:
i) a sialic acid biosynthesis pathway comprising glucosamine 6-phosphate N-acetyltransferase, N-acylglucosamine 2-epimerase, and N-acetylneuraminic acid synthetase, and
ii) a cytidine 5′-monophospho-N-acetylneuraminic acid synthetase (CMP-N-acetylneuraminic acid synthetase or CMP-sialic acid synthetase).

25. The microorganism of claim 19, wherein the sialyltransferase originates from Photobacterium damselae.

26. The microorganism of claim 19, wherein the microorganism
a) has a reduced or abolished expression of at least one polynucleotide encoding or driving expression of a polypeptide that converts i) N-acetylglucosamine-6-P to glucosamine-6-P, ii) N-acetyl-glucosamine to N-acetyl-glucosamine-6-P, or iii) N-acetyl-neuraminate to N-acetyl-mannosamine, or
b) is unable to convert at least one of
i) N-acetylglucosamine-6-P to glucosamine-6-P,
ii) N-acetyl-glucosamine to N-acetyl-glucosamine-6-P, and
iii) N-acetyl-neuraminate to N-acetyl-mannosamine.

27. The microorganism of claim 19, wherein the microorganism has a reduced or abolished activity of at least one enzyme selected from the group consisting of i) a N-acetylglycosamine-6-phosphate deacetylase, ii) a N-acetylglucosamine kinase, and iii) a N-acetylneuraminate aldolase.

28. The microorganism of claim 19, wherein the microorganism comprises: at least one polynucleotide encoding a phosphatase that comprises at least one of:
Motif 1:
(SEQ ID NO: 73)
hDxDx[TV],
or
Motif 2:
(SEQ ID NOs: 74, 75, 76, 77)
[GSTDE][DSEN]x(1-2)[hP]x(1-2)[DGTS]
wherein h is a hydrophobic amino acid (A, I, L, M, F, V, P, or G) and x can be any distinct amino acid,
at least one polynucleotide encoding an N-acetylmannosamine epimerase,
at least one polynucleotide encoding a sialyltransferase,
at least one polynucleotide encoding a CMP-sialic acid synthetase, and
at least one polynucleotide encoding a sialic acid synthase.

29. The microorganism of claim 19, wherein the microorganism comprises a polynucleotide encoding a sialic acid synthase polypeptide originating from Campylobacter jejuni or Neisseria meningitides.

30. The microorganism of claim 19, wherein the microorganism exhibits an increased expression of a phosphatase comprising:
any one of SEQ ID NOs: 58-60, 65, 67, 69, and 70, or
a homologue of the polypeptide of any one of SEQ ID NOs: 42-48, 50-52, 54, 55, 57-60, 65, 67, 69, and 70, having at least 80% overall sequence identity to the polypeptide, which homologue has phosphatase activity, wherein the homologue increases one or more of sialic acid, biomass production and maximal growth rate in the microorganism as compared to a reference strain of the microorganism having the same genetic make-up, but lacking increased expression of the phosphatase.

31. The microorganism of claim 19, wherein the sialylated compound(s) comprise(s) a sialylated oligosaccharide is from the group consisting of sialyllactose, disialyl lacto-N-tetraose, sialylated lacto-N-triose, sialylated lacto-N-tetraose, and sialylated lacto-N-neotetraose.

32. The microorganism of claim 31, wherein the sialylated oligosaccharide is sialyllactose.

33. The microorganism of claim 19, wherein the sialylated compound(s) comprise(s) 3′sialyllactose.

34. The microorganism of claim 19, wherein the sialylated compound(s) comprise(s) 6′sialyllactose.

35. The microorganism of claim 19,
wherein the sialylated compound(s) comprise(s) a sialylated oligosaccharide, which is a sialylated lacto-N-triose, sialylated lacto-N-tetraose or sialylated lacto-N-neotetraose, and
wherein the microorganism further has galactosyltransferase (EC 2.4.1.38) activity, and/or N-acetylglucosaminyltransferase (EC 2.4.1.90) activity.

36. The microorganism according to claim 35, wherein the microorganism is unable to express genes coding for UDP sugar hydrolase and galactose-1-phosphate uridylyltransferase.

37. The microorganism according to claim 28, wherein the N-acetylmannosamine epimerase and/or sialic acid synthase is/are overexpressed in the microorganism or is introduced and expressed in the microorganism.

38. The microorganism of claim 19, wherein the microorganism further:
encodes a protein that facilitates uptake of lactose and
lacks enzymes that metabolize lactose.

39. The microorganism of claim 19, wherein the microorganism is a bacterium or a yeast.

40. The microorganism of claim 19, wherein the microorganism has at least one disabled polypeptide of the phosphoenolpyruvate:sugar phosphotransferase system for the import of a saccharide that is not used as a carbon source during fermentative production of the sialylated compounds.

41. The microorganism according to claim 40, wherein the at least one disabled polypeptide of the phosphoenolpyruvate: sugar phosphotransferase system is encoded by at least one of the genes selected from the group of genes encoding manX, manY, manZ, or nagE.

42. The microorganism of claim 19, wherein the microorganism can utilize an exogenous carbon source present in fermentation broth as sole carbon source without using a phosphoenolpyruvate: sugar phosphotranferase system for the acquisition of the exogenous carbon source.
